(19) United States
(12) Patent Application Publication
(10) Pub. No.: US 2019/0388510 A1
(43) Pub. Date: Dec. 26, 2019

(54) GLUCAGON-T3 CONJUGATES

(71) Applicant: INDIANA UNIVERSITY RESEARCH AND TECHNOLOGY CORPORATION, Indianapolis, IN (US)

(72) Inventors: Richard D. DIMARCHI, Carmel, IN (US); Brian FINAN, Indianapolis, IN (US); Bin YANG, Plainfield, IN (US); Zhimeng ZHU, Wuhan, Hubei (CN)

(21) Appl. No.: 16/302,795

(22) PCT Filed: May 26, 2017
(86) PCT No.: PCT/US2017/034617

§ 371 Date: Nov. 19, 2018

Related U.S. Application Data
(60) Provisional application No. 62/344,664, filed on Jun. 2, 2016.

(51) Int. Cl. A61K 38/26; A61K 47/55; A61P 3/06; A61P 3/04; A61P 9/10; A61K 31/198

(52) U.S. Cl. CPC A61K 38/26; A61K 47/55; A61K 31/198; A61P 3/04; A61P 9/10; A61P 3/06

(57) ABSTRACT

Provided herein are glucagon agonist peptides conjugated with thyroid hormone receptor ligands that are capable of acting at the thyroid hormone receptor. Also provided herein are pharmaceutical compositions and kits of the conjugates of the invention. Further provided herein are methods of treating a disease, e.g., a metabolic disorder, such as diabetes, obesity, metabolic syndrome and chronic cardiovascular disease, comprising administering the conjugates of the invention.

### CROSS REFERENCE TO RELATED APPLICATIONS

This application claims priority to U.S. Provisional Patent Application No. 62/344,664 filed on Jun. 2, 2016, the disclosure of which is hereby expressly incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 531 kilobytes acii (text) file named “265652seqlist_ST25.txt,” created on May 18, 2017.

### BACKGROUND

Homeostatic control of plasma and cellular lipids is crucial for maintaining proper health. Dyslipidemia, including hypercholesterolemia and hypertriglyceridemia, represents a hallmark of the metabolic syndrome and triggers a host of obesity comorbidities. Coordinated impairments in hepatic lipid metabolism and diminished capacity of adipocytes to properly store lipids lead to lipid spillover with ectopic fat deposition in susceptible organs. Liver and adipose tissues orchestrate systemic lipid homeostasis and reciprocal dysfunction in these organs propels a vicious cycle of metabolic derangements. Notably, hepatic steatosis is a key pathogenic factor in hepatic insulin resistance and non-alcoholic fatty liver disease (NAFLD), and perturbed cholesterol handling accelerates atherosclerosis, thus positioning dyslipidemia at the interface of type 2 diabetes (T2D) and coronary heart disease (CHD). Importantly, CHD remains the leading cause of death globally and T2D is now recognized as an independent CHD-risk-equivalent condition.

Inhibiting hepatic cholesterol synthesis via statins provides clinically relevant reductions in circulating cholesterol and proportionally lowers CHD risk. However, a considerable number of patients still fail to meet target reductions in cholesterol. Furthermore, a significant limitation of statin therapy is its near exclusive focus on cholesterol lowering with no benefit to glycemic control or body weight. Therefore, a pharmacological agent that lowers cholesterol, triglycerides, glucose, hepatic fat and body weight would offer a transformative advancement for treatment of the metabolic syndrome that should decrease mortality risk from cardiovascular events.

Thyroid hormones powerfully influence systemic metabolism through multiple pathways, with profound effects on energy expenditure, fat oxidation, and cholesterol metabolism. Clinical reports revealed sixty years ago that administration of thyroid extracts reduced circulating cholesterol and reversed obesity. However, adverse side effects of thyroid hormone treatment include increased heart rate, cardiac hypertrophy, muscle wasting, and reduced bone density, terminating its clinical use.

Discovery of thyromimetics capable of separating lipid metabolism benefits from adverse cardiovascular effects has remained a desire for patients, physicians and the pharmaceutical industry. Human genomic data and studies in isoform-specific knockout mice have suggested that thyroid hormone receptor alpha (TRα) mediates the hypertrophic cardiovascular actions of thyroid hormones while thyroid hormone receptor beta (TRβ) promotes hepatic lipid metabolism, and both isoforms mediate lipolysis and thermogenesis in adipose tissues. This knowledge has initiated attempts to rationally design small molecules with selective preference for TRβ compared to TRα for the purpose to treat dyslipidemia. Second generation thyromimetics sought isoform specificity and tissue-specific function by derivatization with chemical moieties to promote tissue selectivity. These functionalized adducts sought to promote the interaction with liver-specific transporters or were designed to take advantage of hepatic first-pass metabolism to release an active thyromimetic. These liver-targeted thyromimetics initially showed promising pre-clinical effects on handling hepatic lipids and atherogenic lipoproteins.

Glucagon is classically known as the insulin-opposing hormone that induces hepatic glucose production to buffer against hypoglycemia and maintain proper glucose homeostasis. Exogenous glucagon administration also offers many benefits for metabolic diseases independent from its glycemic effects. Studies more than 50 years ago first demonstrated liver-mediated effects of glucagon to lower circulating cholesterol and triglycerides in rodents and humans. Furthermore, glucagon directly influences hepatic fat metabolism. The benefits of glucagon action are not solely constrained to the liver as adipose tissue is a secondary target organ for glucagon action. In white adipose tissue, glucagon promotes lipolysis and increases energy expenditure through thermogenic mechanisms. These lipolytic and thermogenic actions demonstrate the validity of glucagon-based agonists as an anti-obesity therapy, but only if the inherent diabetogenic liability can be properly controlled.

In accordance with the current disclosure, compositions are provided wherein the liver-mediated lipid lowering properties of glucagon, as well as the adipose-mediated thermogenic properties of glucagon are combined with thyroid hormone activity in a single complex. Liver directed T3 action offsets the diabetogenic liability of glucagon, and glucagon-mediated delivery spares the cardiovascular system from adverse T3 action. The therapeutic utility of glucagon and thyroid hormone pairing provides a new approach in treatment of obesity, type 2 diabetes, and cardiovascular disease.

### SUMMARY

Applicants disclose compositions and methods for glucagon-mediated selective delivery of thyroid hormone action to the liver as a primary target and to inguinal white fat (iWAT) as a secondary target. Together, coordinated glucagon and thyroid hormone actions synergize to correct hyperlipidemia, reverse hepatic steatosis and lower body weight through liver and fat-specific mechanisms. Importantly, the liver-directed thyroid hormone action overrides the diabetogenic liability of local glucagon action resulting in a net improvement of glycemic control, while glucagon-mediated delivery spares adverse action of thyroid hormone on the cardiovascular system.

Provided herein are chemical conjugates of a glucagon agonist peptide and compounds having thyroid hormone activity (“glucagon/T3 conjugates”). These conjugates with plural activities are useful for the treatment of a variety of diseases including hyperlipidemia, metabolic syndrome, diabetes, obesity, liver steatosis, and chronic cardiovascular disease. Advantageously, the disclosed conjugates lack the adverse effects on the cardiovascular system that are associated with T3 administration and also lack the adverse effect of elevated blood glucose that are associated with the administration of glucagon. The glucagon/T3 conjugates of the present disclosure can be represented by the following formula:

Q-L-Y

wherein Q is a glucagon agonist peptide, Y is a thyroid hormone receptor ligand, and L is a linking group or a bond. In accordance with one embodiment Q is a glucagon agonist peptide that exhibits agonist activity at the glucagon receptor. In some embodiments, the glucagon agonist peptide is a fusion peptide wherein a second peptide has been fused to the C-terminus of the glucagon peptide. The thyroid hormone receptor ligand, (Y) is wholly or partly non-peptide and acts at the thyroid receptor. In some embodiments Y is a compound having the general structure

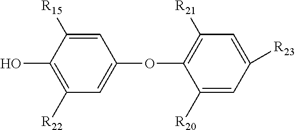

wherein

R₁₅ is C₁-C₄ alkyl, —CH₂(C₆ heteroaryl), —CH₂(OH)(C₆ aryl)F, —CH(OH)CH₃, halo or H

R₂₀ is halo, CH₃ or H;

R₂₁ is halo, CH₃ or H;

R₂₂ is H, OH, halo, —CH₂(OH)(C₆ aryl)F, or C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, —OCH₂PO₃²⁻, —NHC(O)CH₂COOH, OH, halo or C₁-C₄ alkyl. In one embodiment Y is a compound selected from the group consisting of thyroxine T4 (3,5,3′,5′-tetra-iodothyronine), and 3,5,3′-triiodo L-thyronine.

In one embodiment the glucagon agonist peptide (Q) comprises the sequence

X₁X₂X₃GTFTSDYSX₁₂YLX₁₅SRRAQX₂₁FVX₂₄WLX₂₇X₂₈X₂₉ (SEQ ID NO: 925)

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, amino isobutyric acid (Aib), Val, or α-amino-N-butyric acid;

X₃ is an amino acid comprising a side chain of Structure I, II, or III:

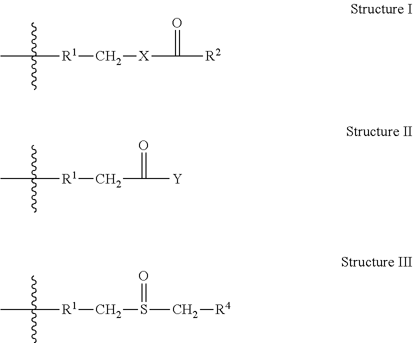

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, O, or S; and Y is NHR⁴, SR³, or OR³;

X₁₂ is Lys or Arg;

X₁₅ is Asp, Glu, cysteic acid, homoglutamic acid or homocysteic acid;

X₂₁ is Asp, Lys, Cys, Orn, homocysteine or acetyl phenylalanine;

X₂₄ is Gln, Lys, Cys, Orn, homocysteine or acetyl phenylalanine;

X₂₇ is Met, Leu or Nle;

X₂₈ is Asn, Lys, Arg, His, Asp or Glu; and

X₂₉ is Thr, Lys, Arg, His, Gly, Asp or Glu, optionally wherein SEQ ID NO: 925 is further modified by one, two, three, or all of the amino acids at positions 16, 20, 21, and 24 being substituted with an α,α-disubstituted amino acid.

In some aspects of the invention, pharmaceutical compositions comprising the Q-L-Y conjugate and a pharmaceutically acceptable carrier are also provided.

In other aspects of the present disclosure, methods are provided for administering a therapeutically effective amount of a Q-L-Y conjugate described herein for treating a disease or medical condition in a patient. In some embodiments, the disease or medical condition is selected from the group consisting of metabolic syndrome, diabetes, obesity, liver steatosis, and chronic cardiovascular disease. In one embodiment the glucagon-T3 conjugates are administered to a patient to treat metabolic syndrome and lipid abnormalities of the liver, including for example non-alcoholic steatohepatitis (NASH).

In one embodiment the therapeutic index of the glucagon-T3 conjugates is enhanced by linking a self-cleaving dipeptide to the active site of the glucagon agonist peptide or the thyroid hormone receptor ligand component of the conjugate. Subsequent removal of the dipeptide under physiological conditions and in the absence of enzymatic activity restores full activity to the Q-L-Y conjugate. Advantageously, the dipeptide will chemically cleave (in the absence of enzymatic activity) under physiological conditions at a rate determined in part by the substituents on the dipeptide. In one embodiment the conjugate Q-L-Y is modified by the covalent linkage of one or more dipeptides (A-B) to an amine of Q or Y, wherein A is an amino acid or a hydroxy acid and B is an N-alkylated amino acid linked to Q or Y through an amide bond between a carboxyl moiety of B and an amine of Q and/or Y. In one embodiment both Q and Y are linked to a dipeptide A-B. In one embodiment, A-B comprises the structure:

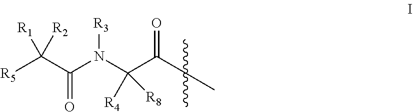

wherein

(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;

(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;

(c) R⁵ is NHR⁶ or OH;

(d) R⁶ is H, C₁-C₈ alkyl; and

(e) R⁷ is selected from the group consisting of H and OH

wherein the chemical cleavage half-life (t_{1/2}) of A-B from Q and/or Y is at least about 1 hour to about 1 week in PBS under physiological conditions.

### BRIEF DESCRIPTION OF THE DRAWINGS

FIGS. 1A-1K. Glucagon/T3 Improves Dyslipidemia and Ameliorates Atherosclerosis in Western Diet-Fed Mice

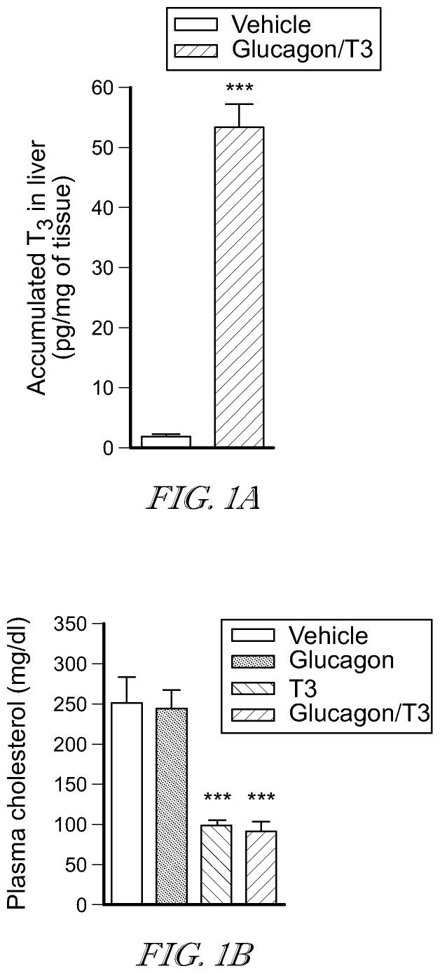

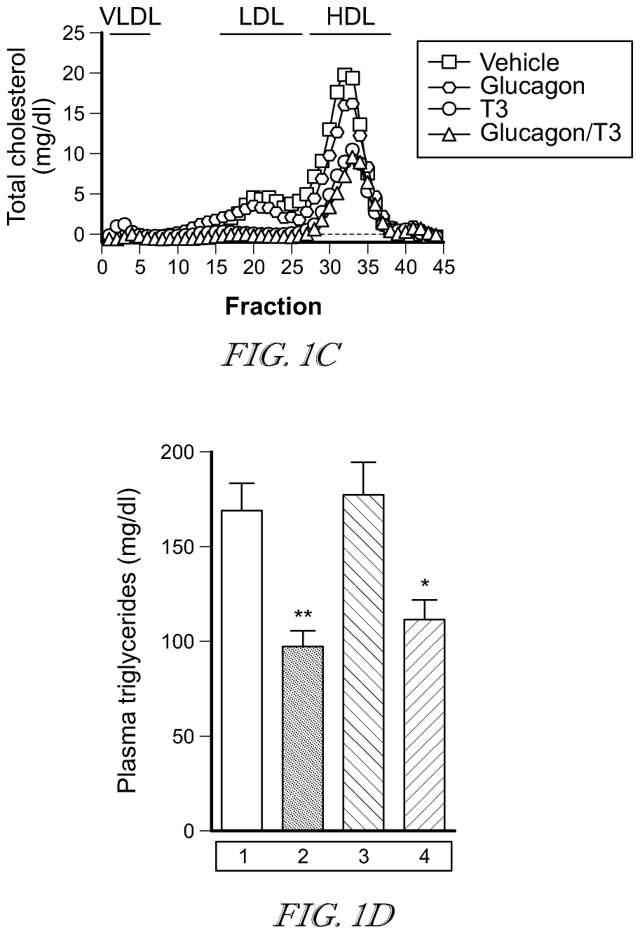

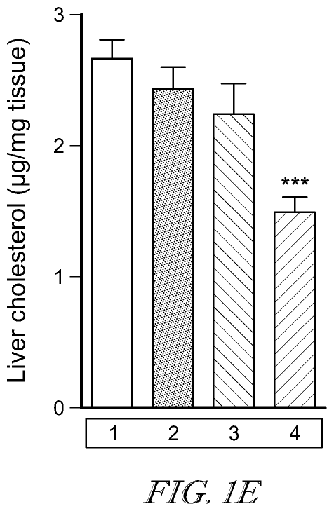

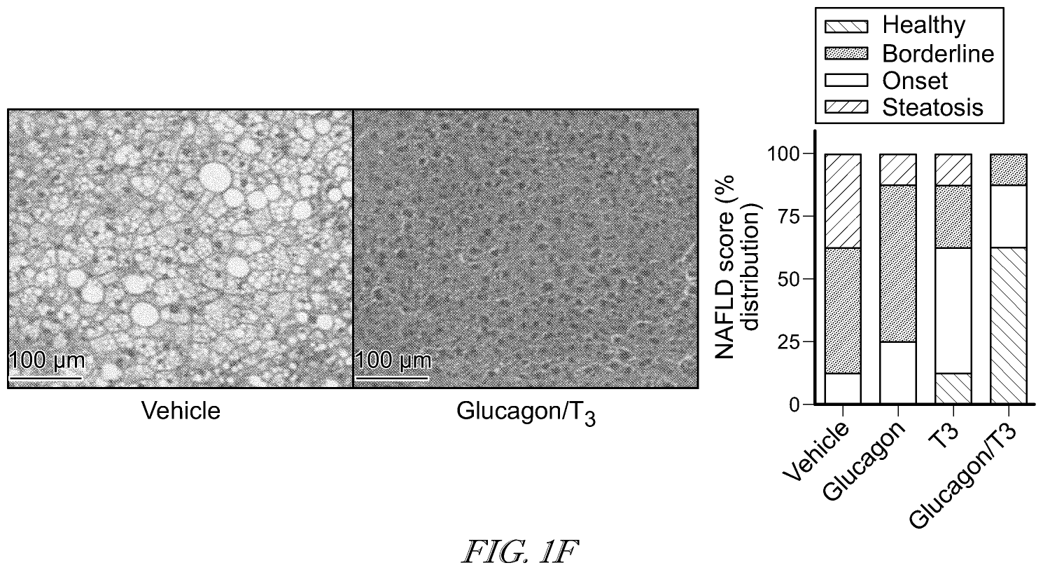

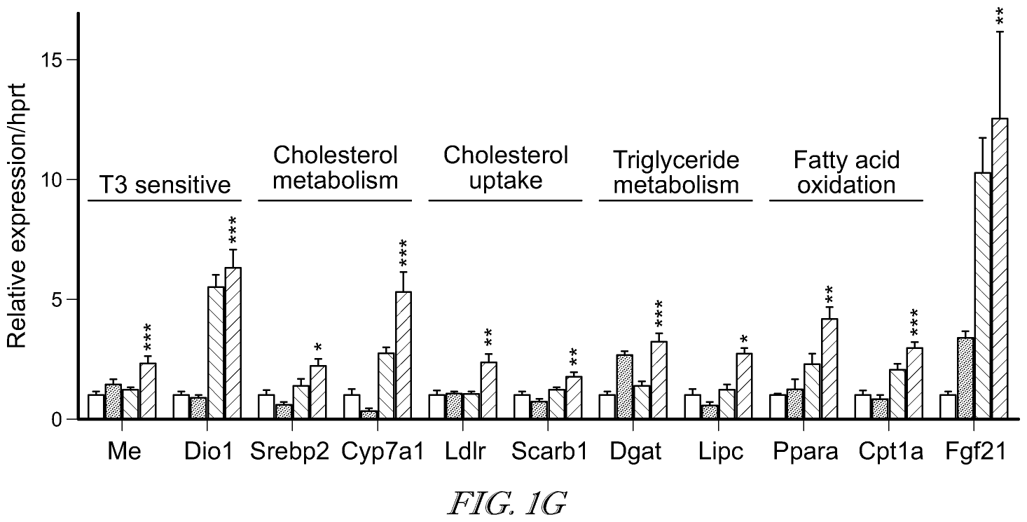

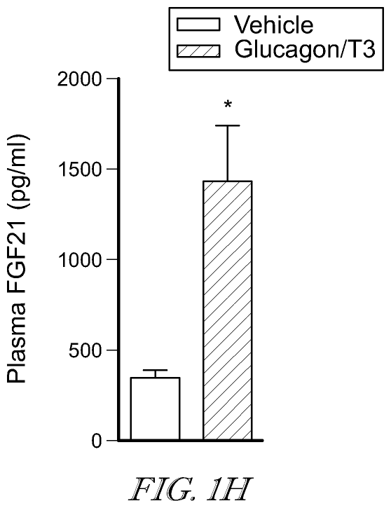

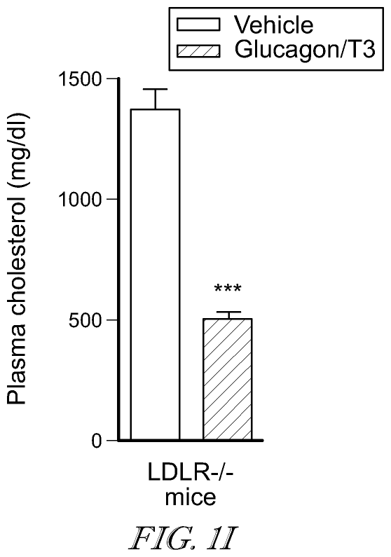

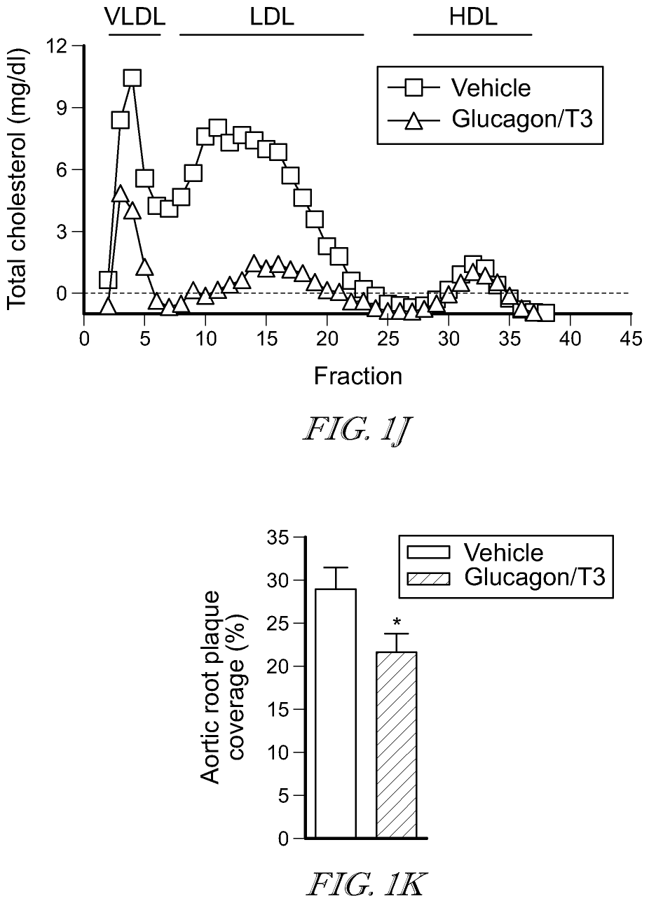

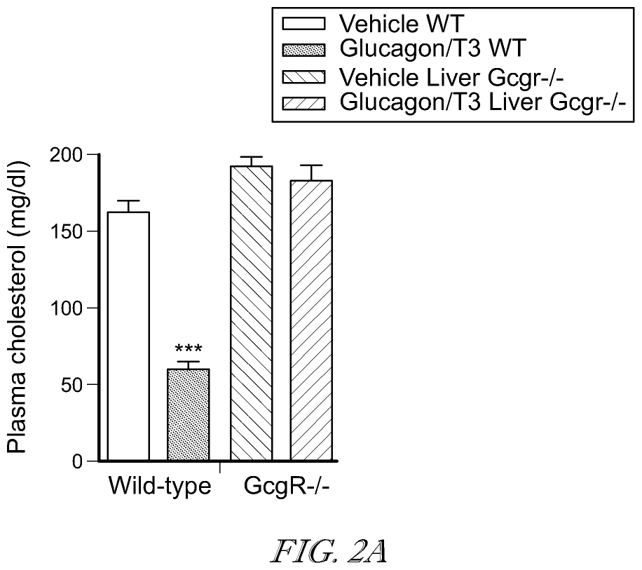

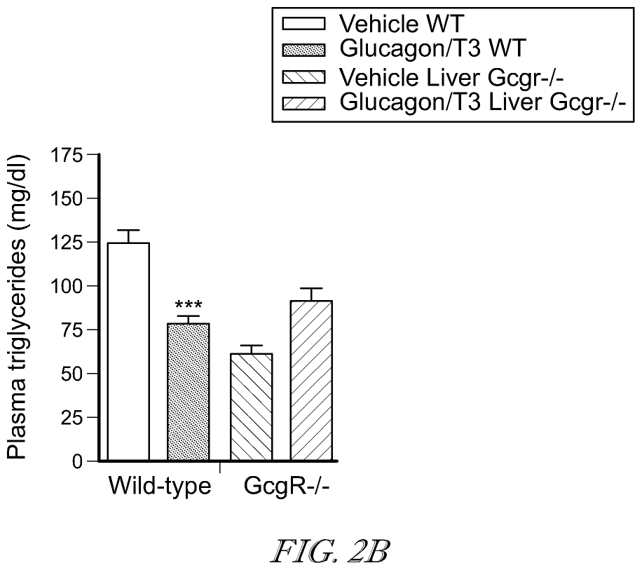

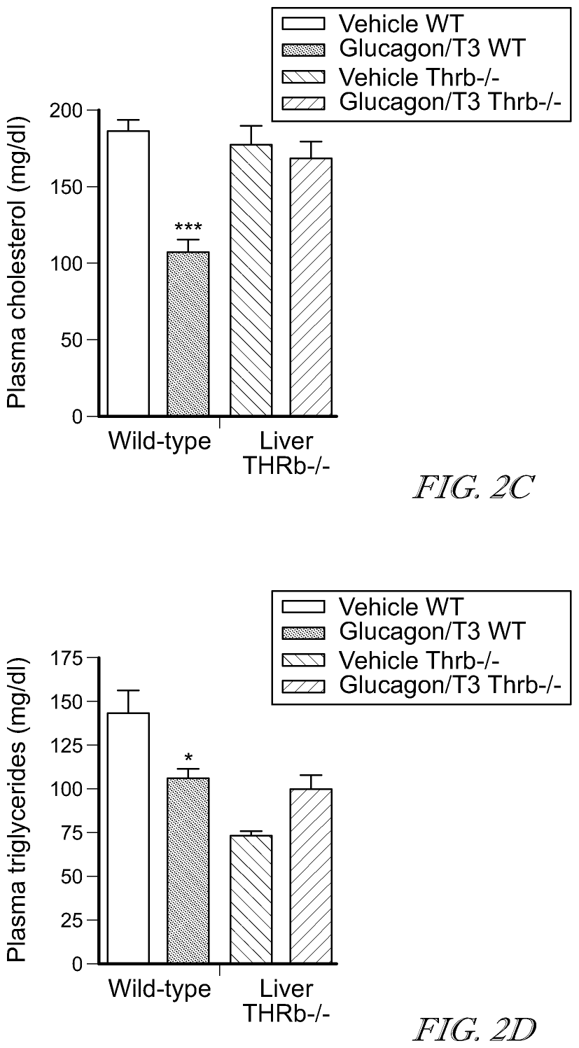

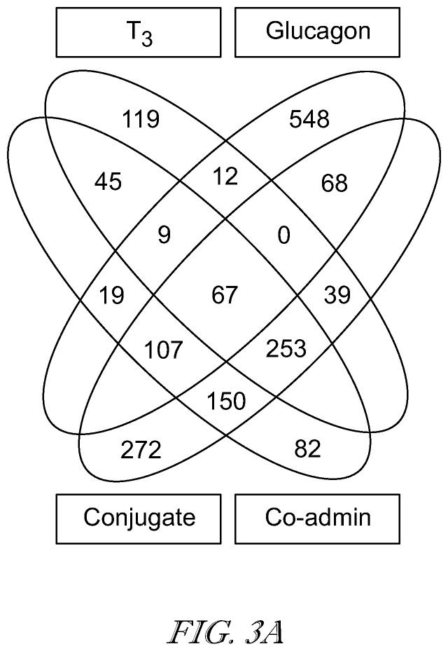

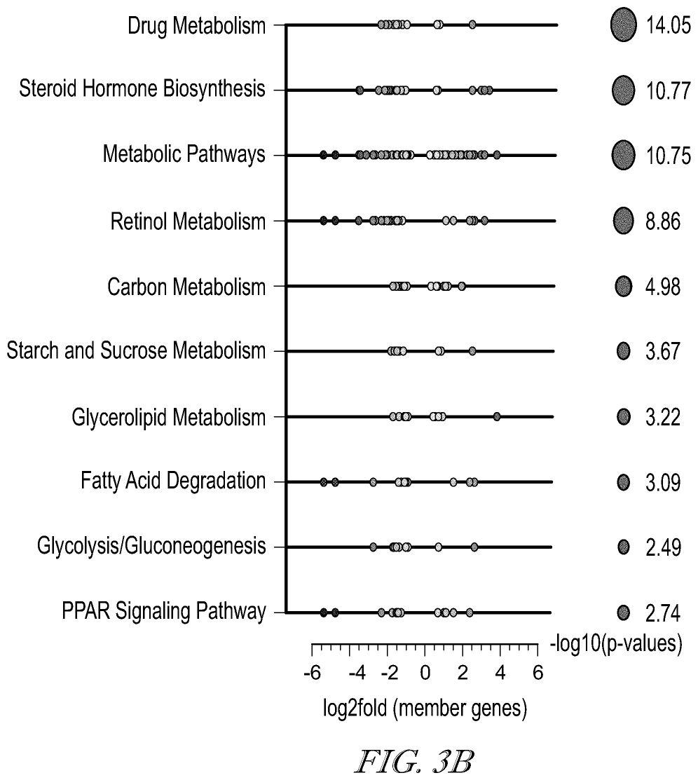

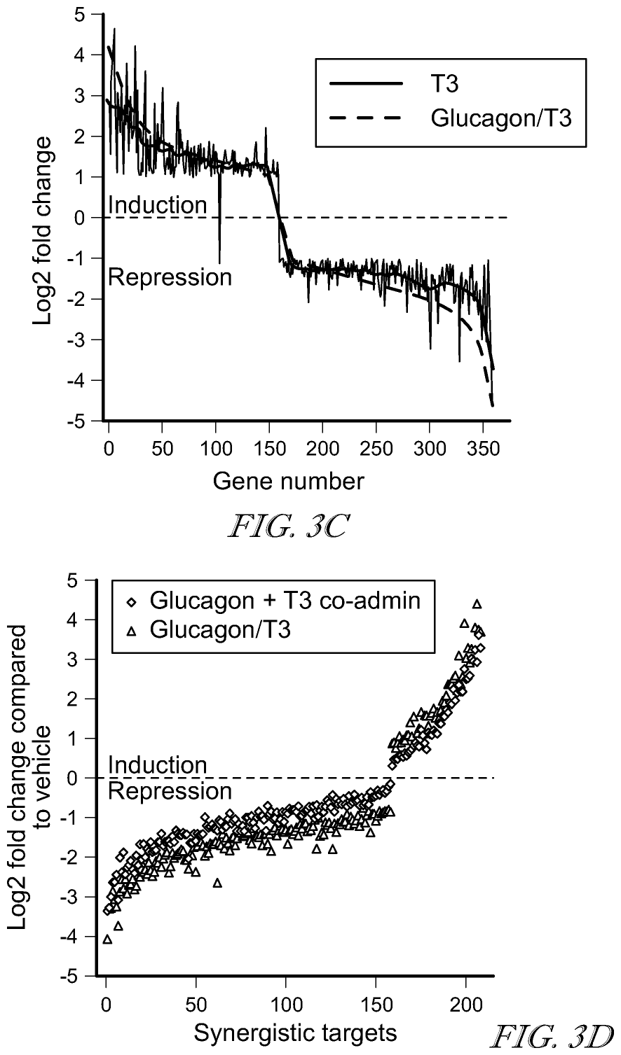

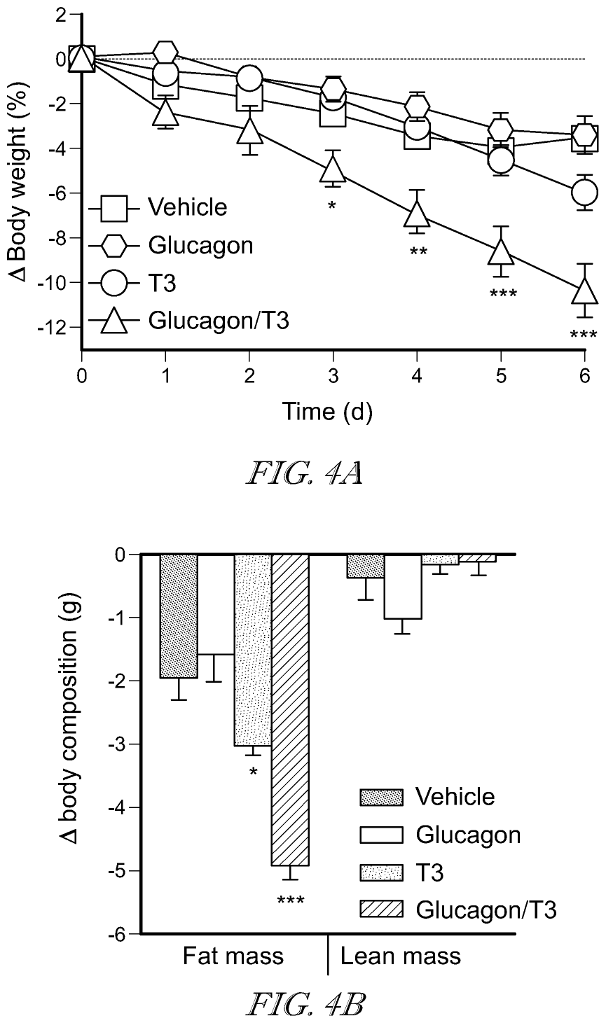

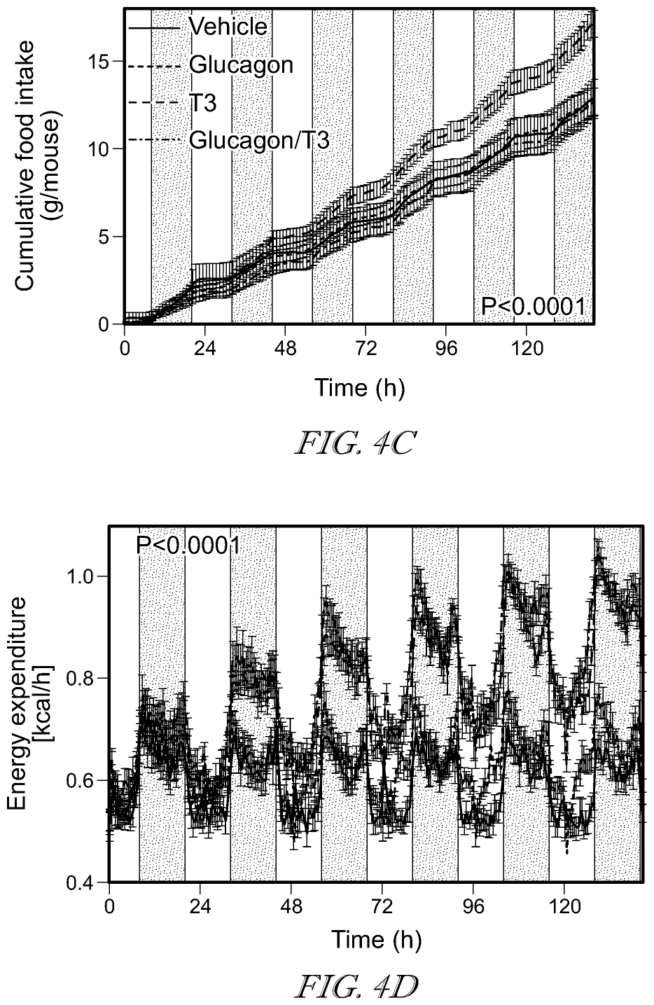

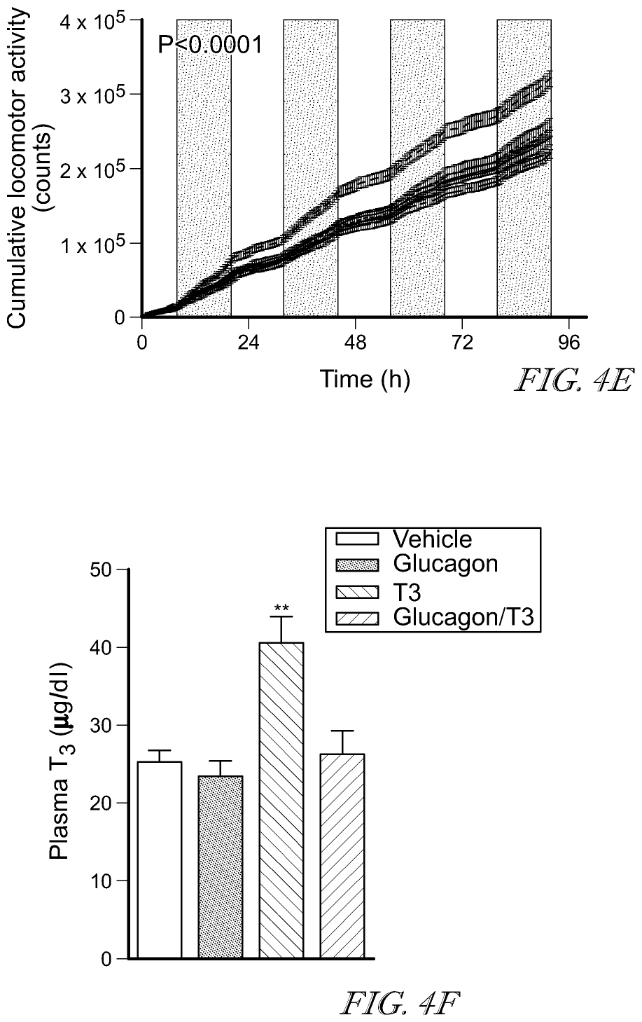

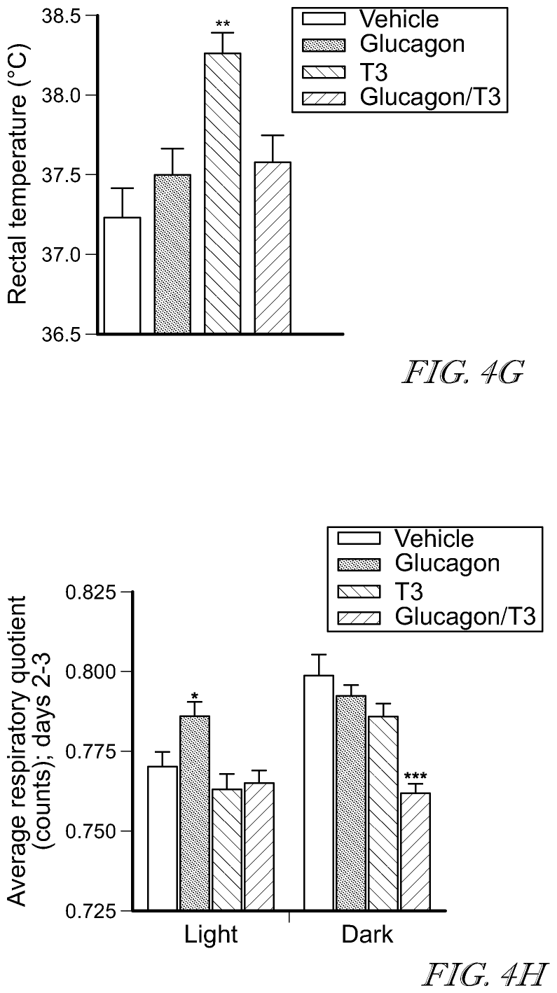

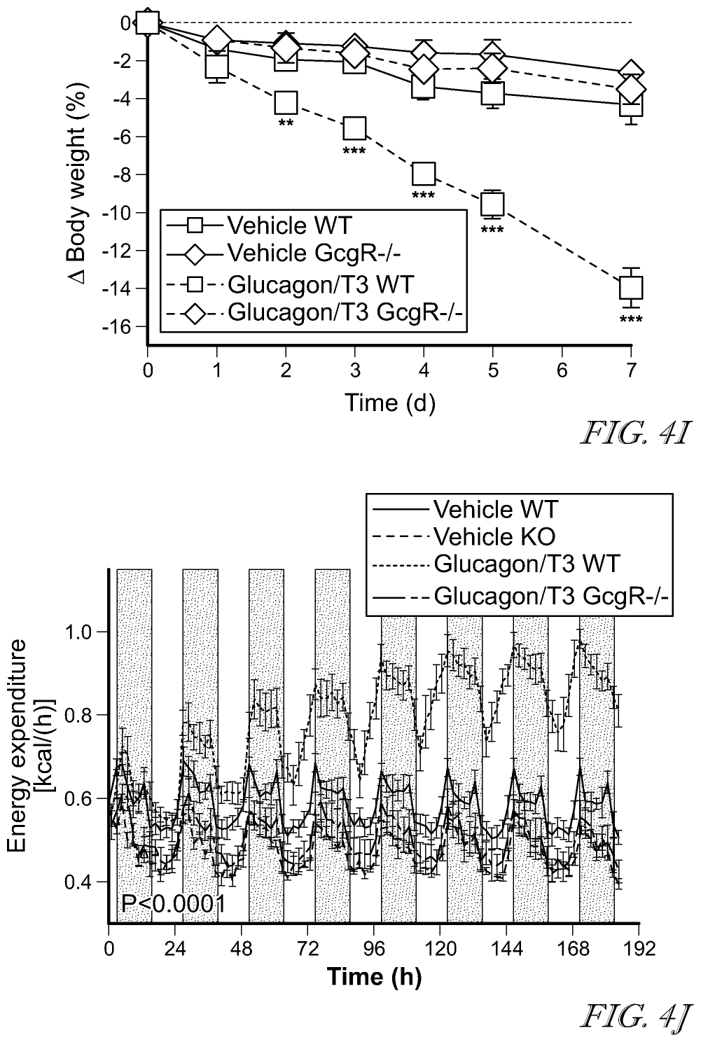

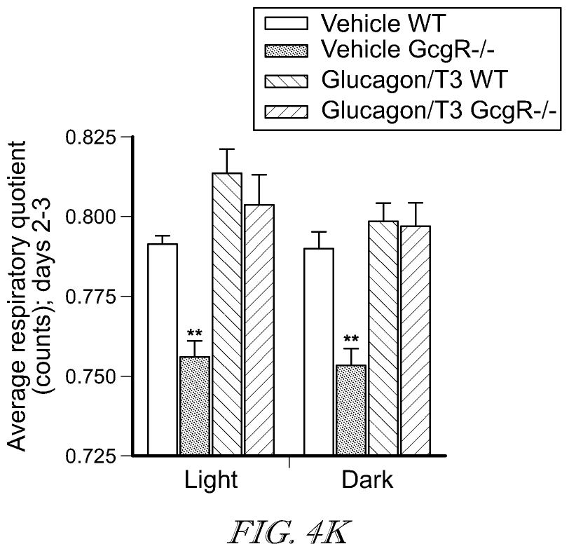

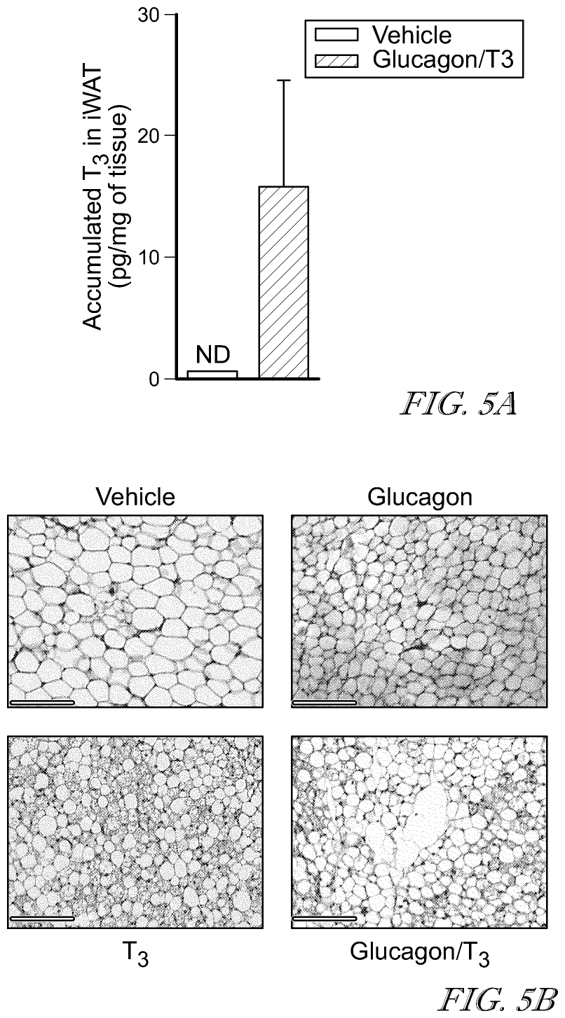

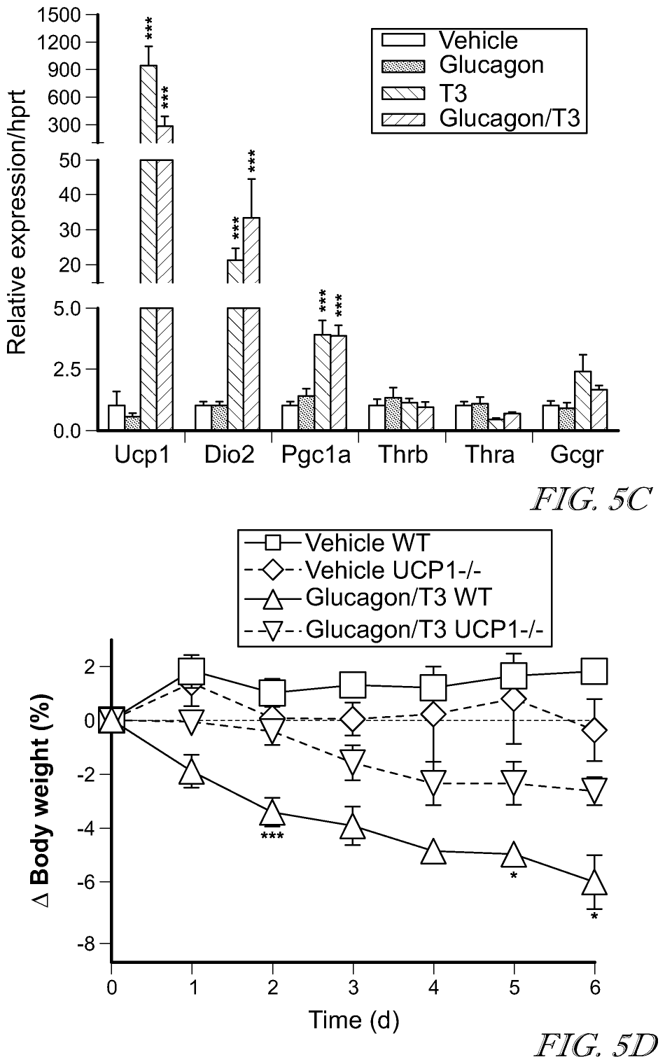

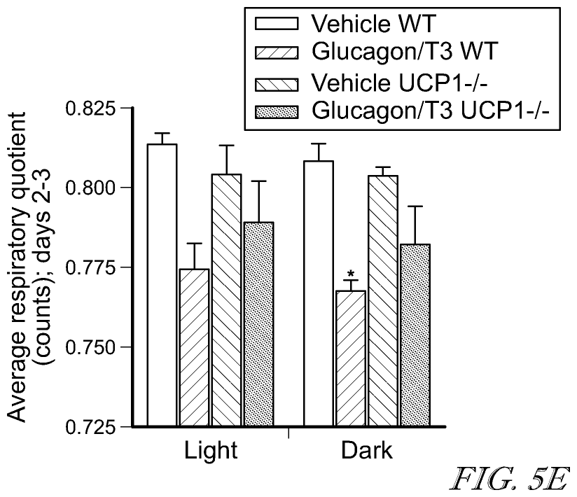

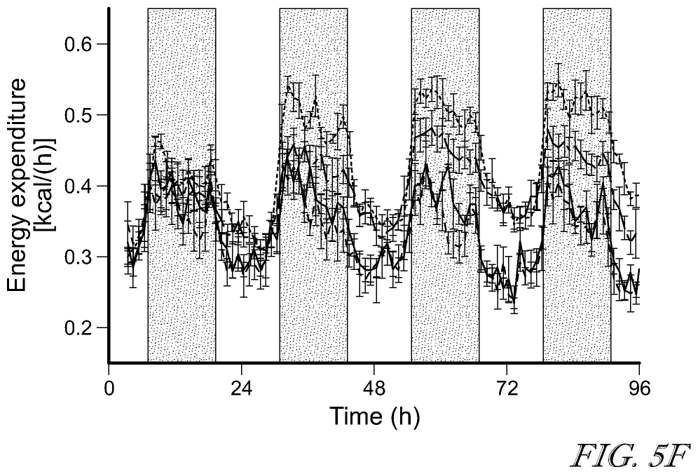

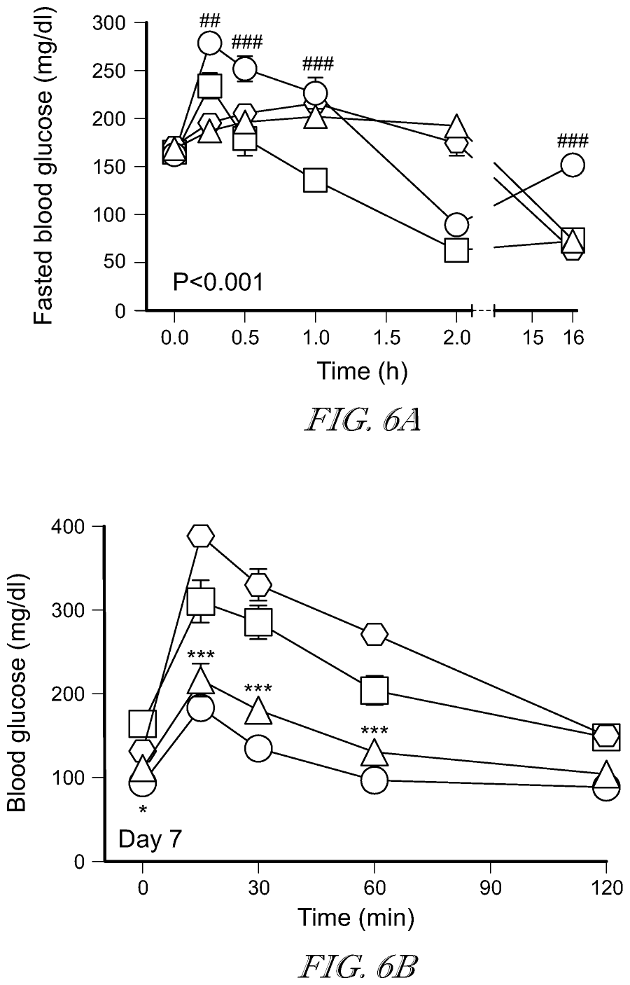

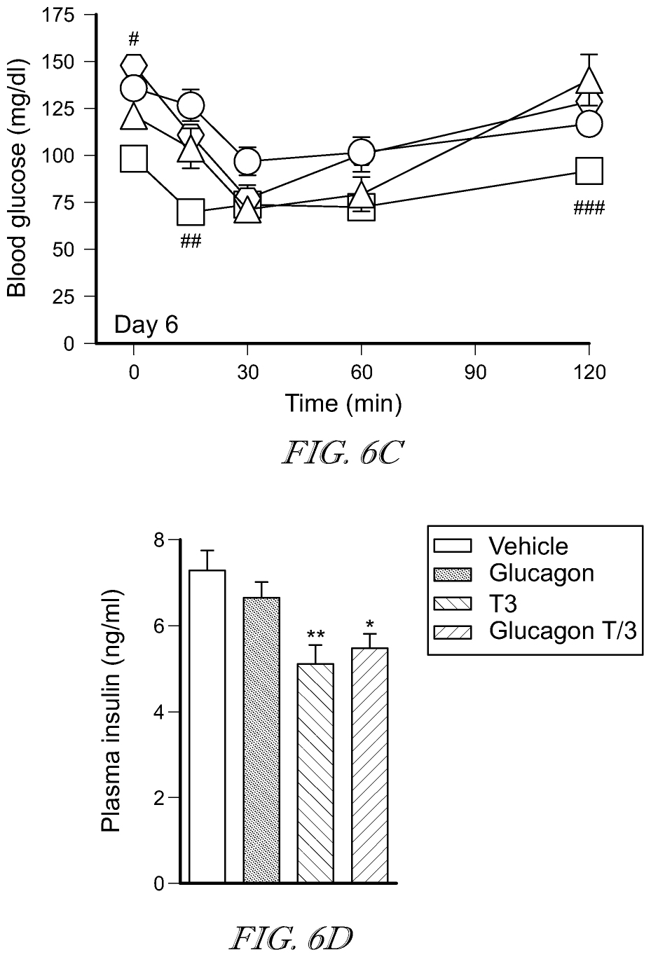

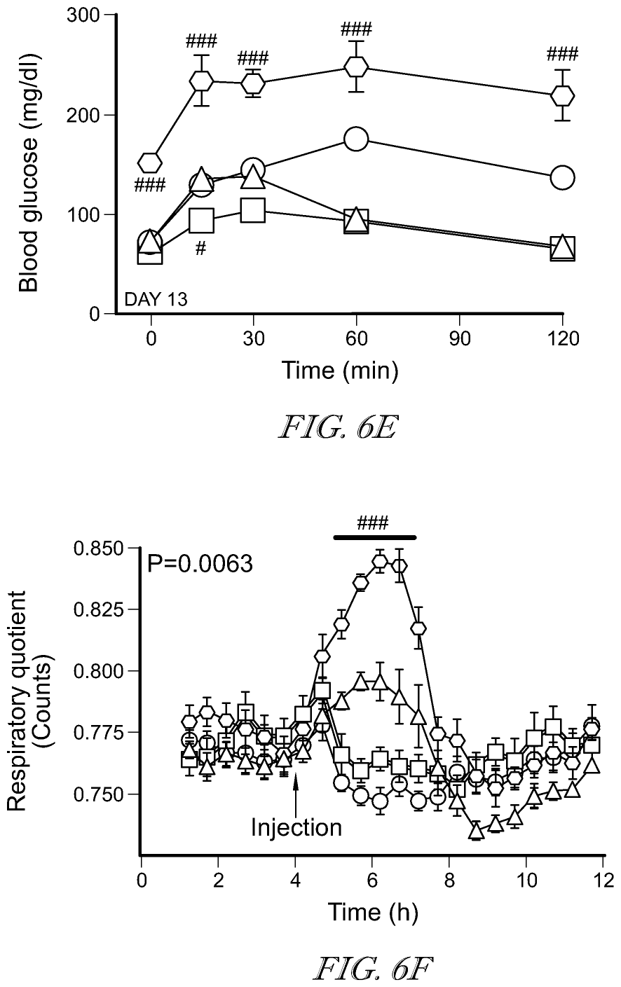

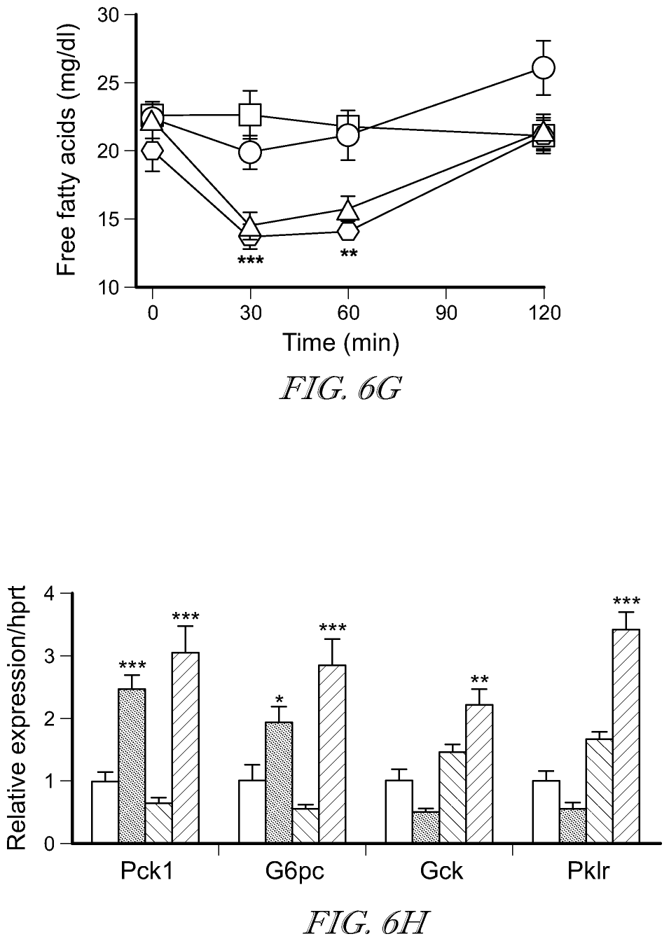

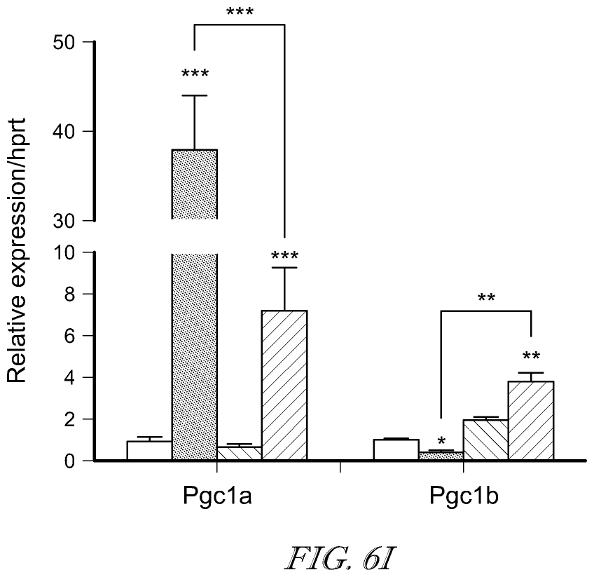

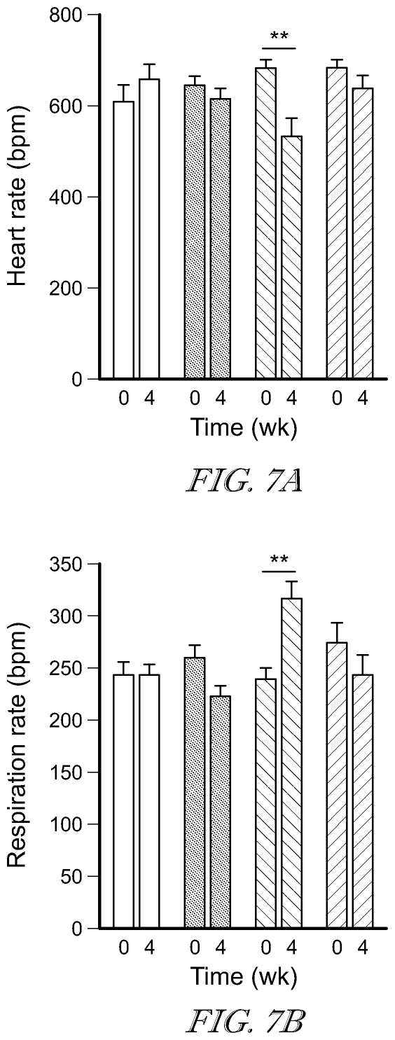

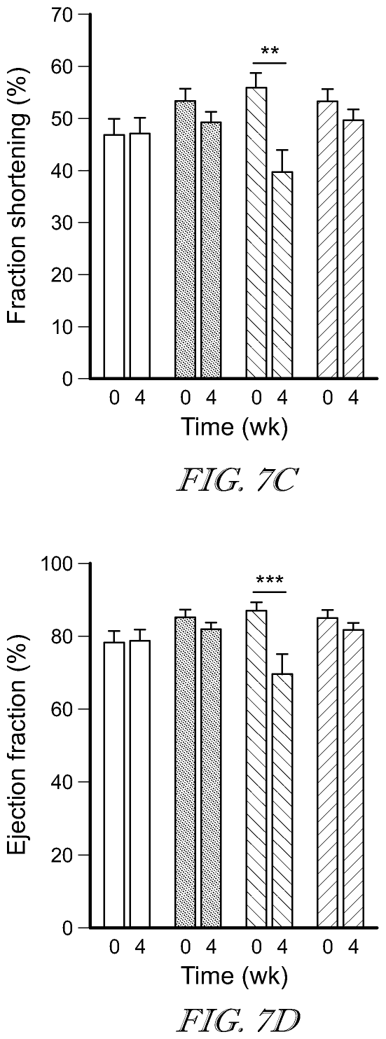

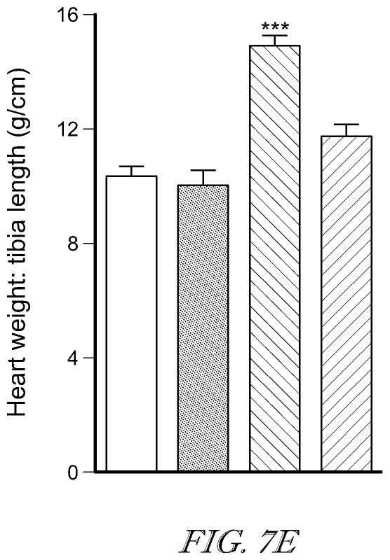

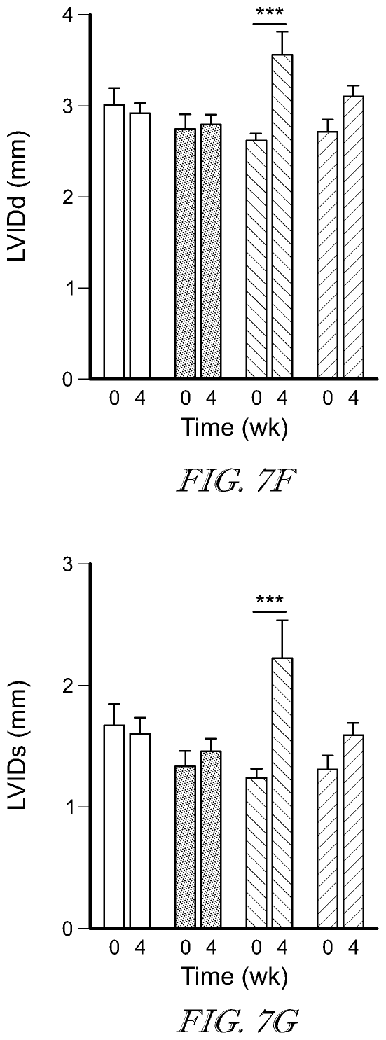

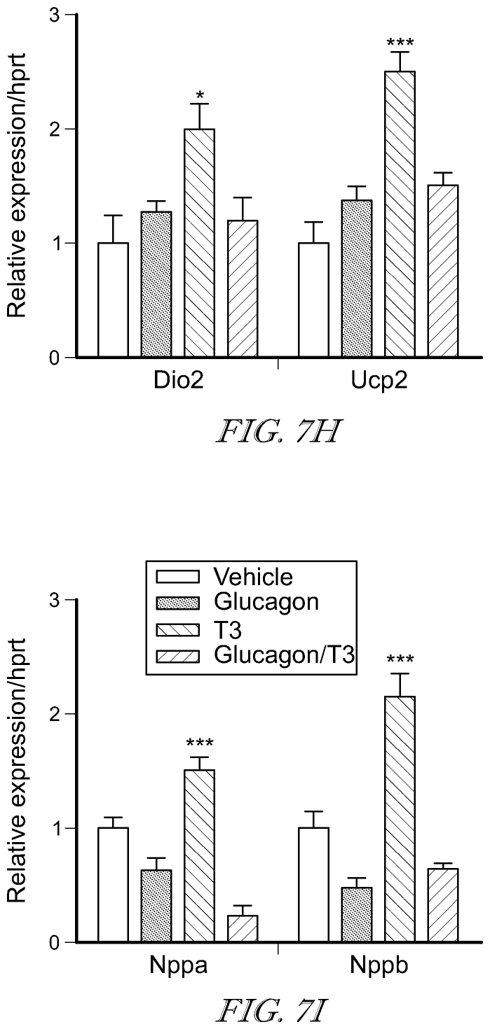

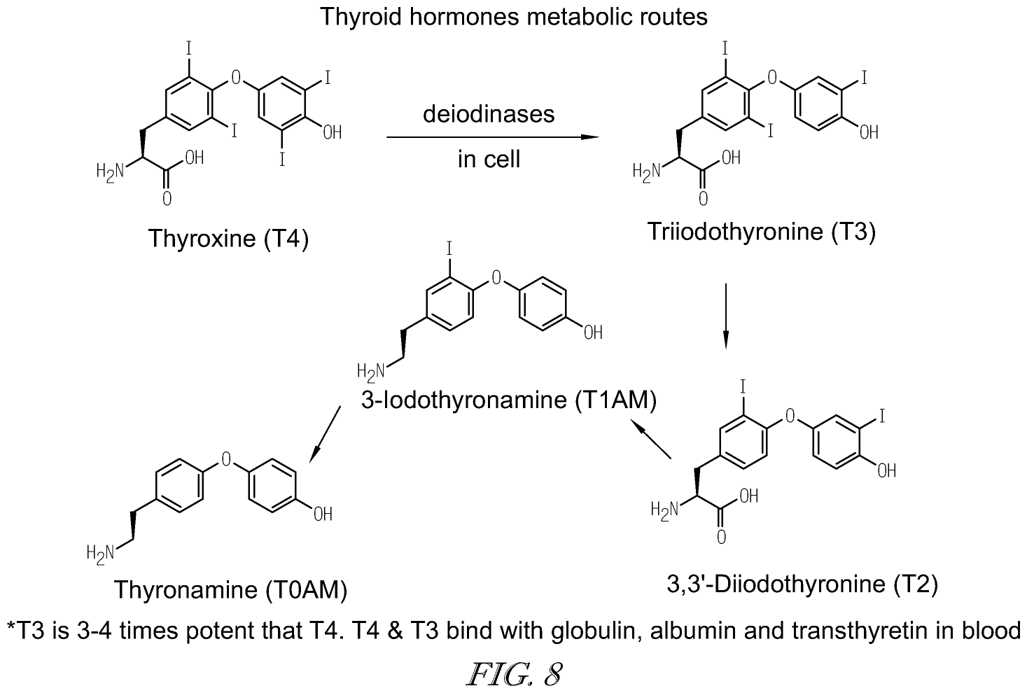

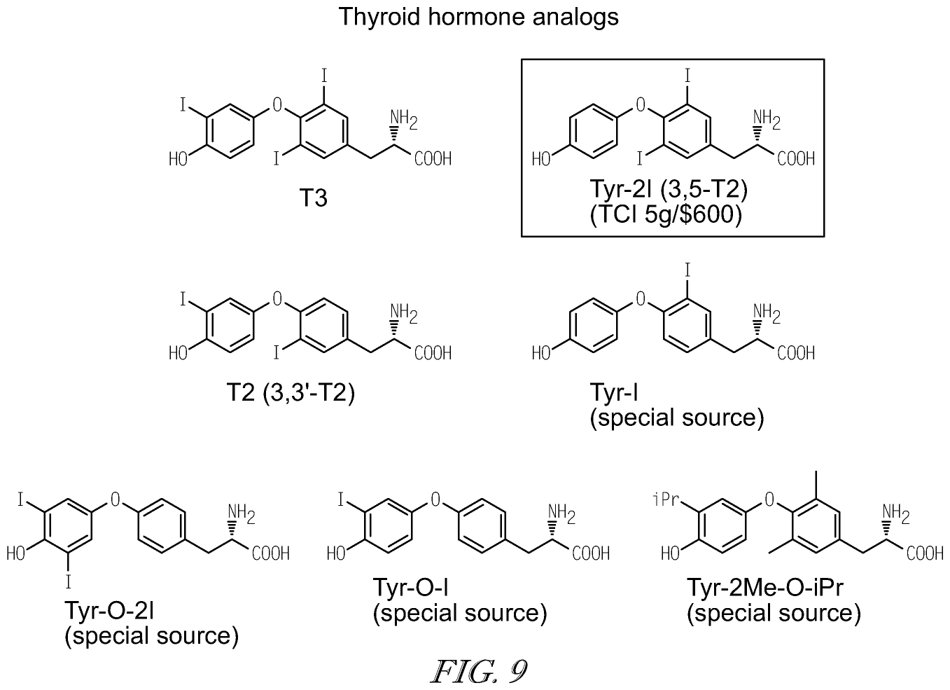

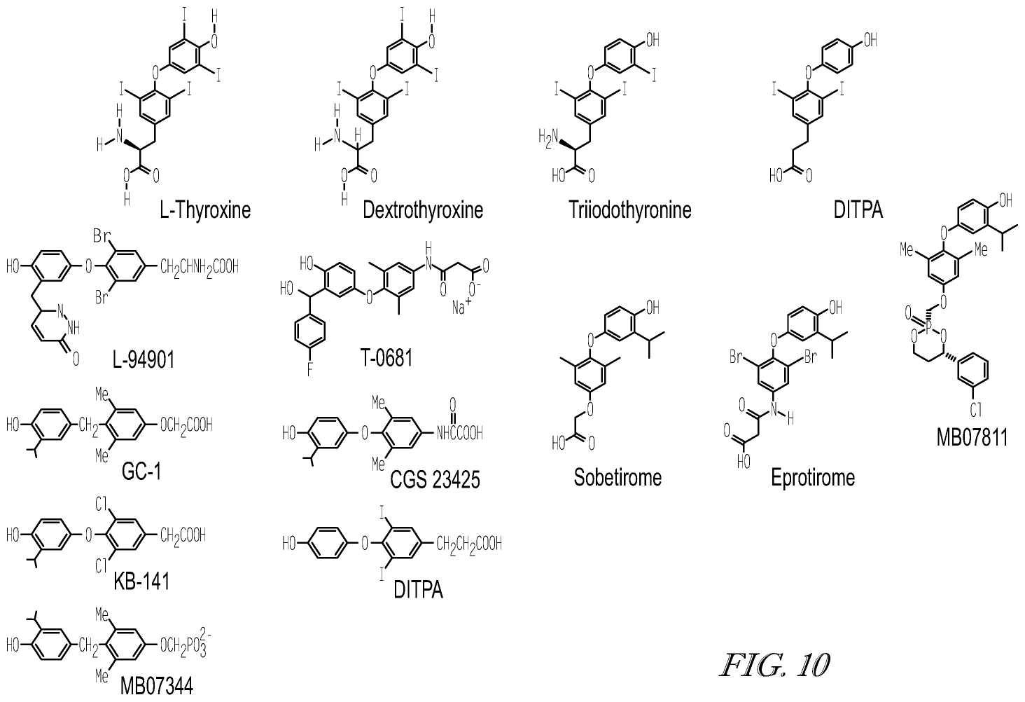

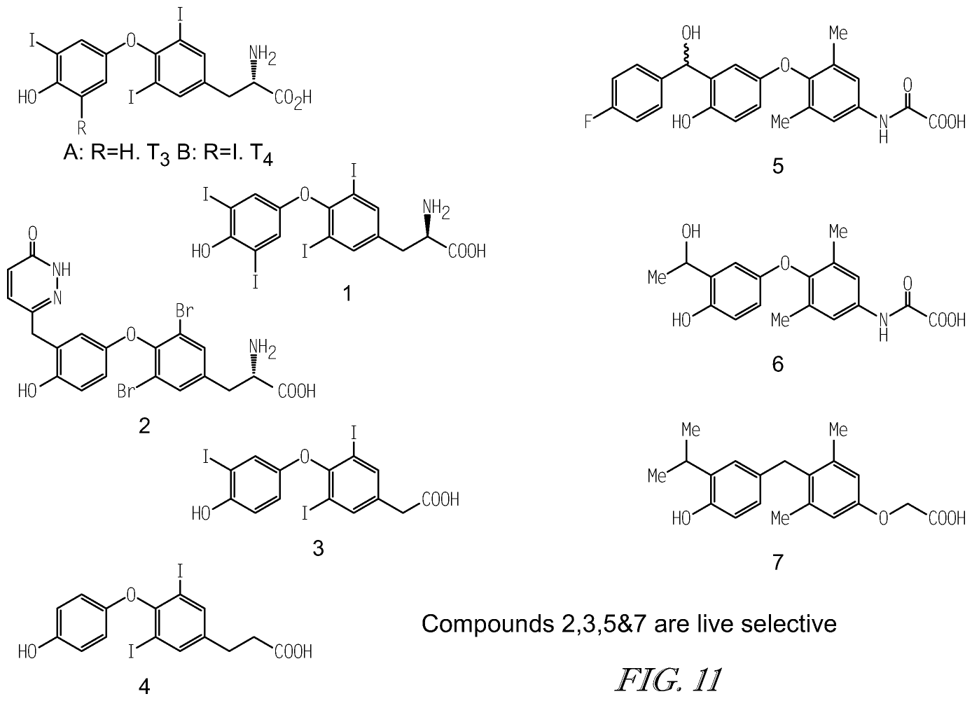

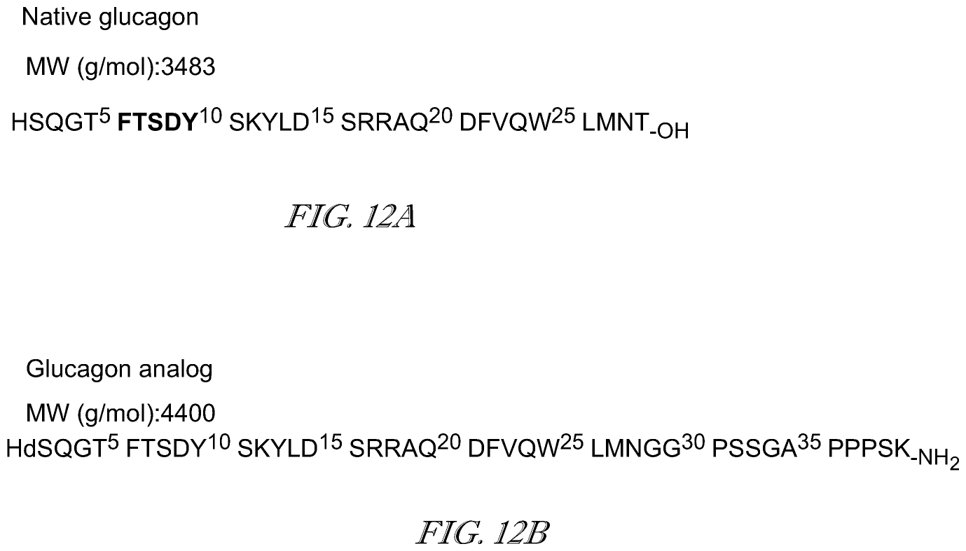

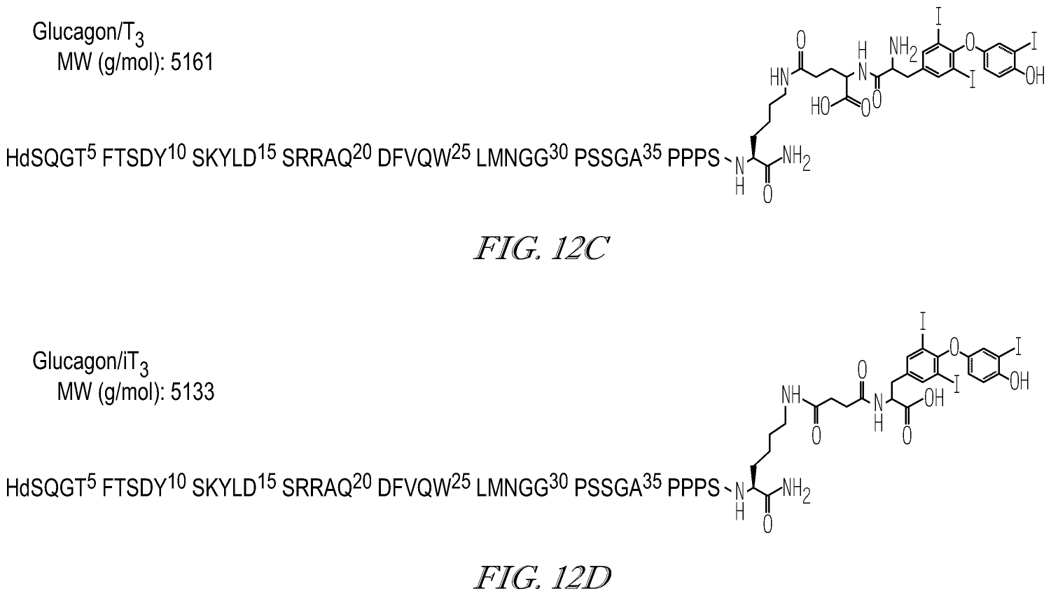

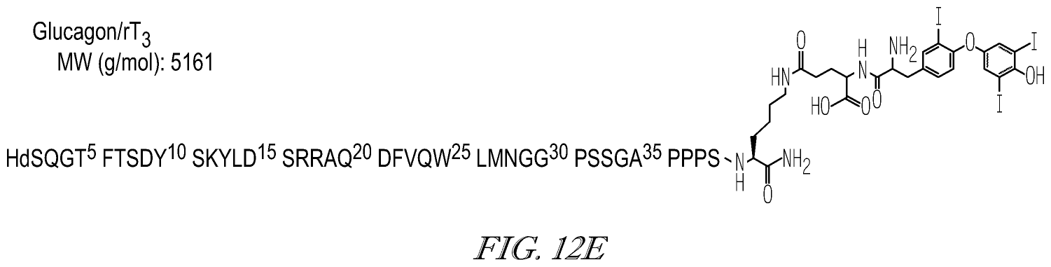

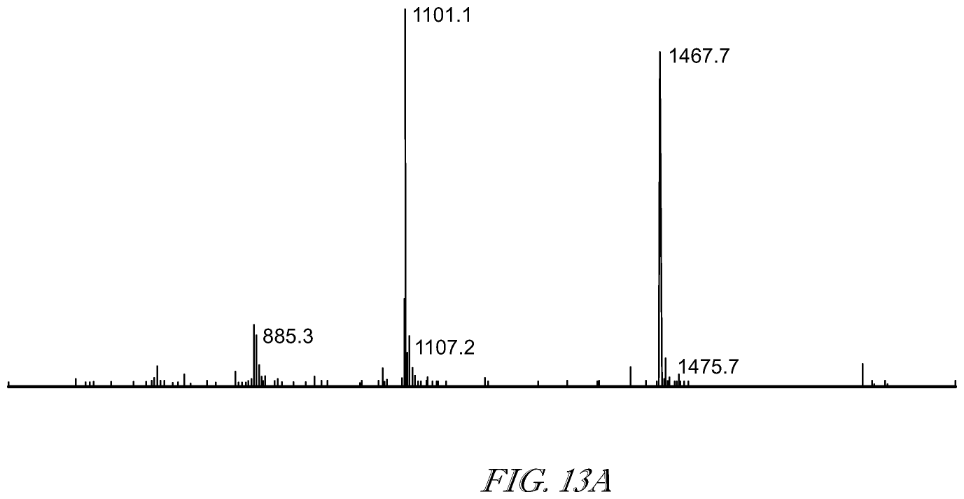

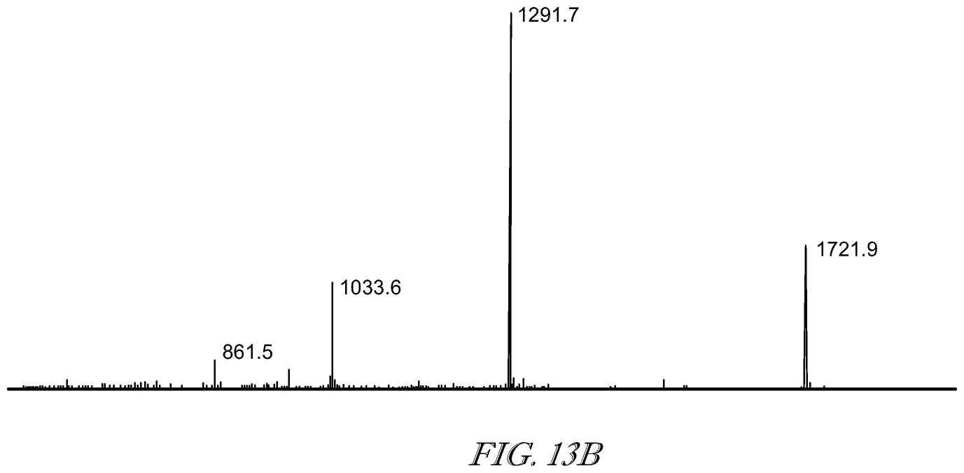

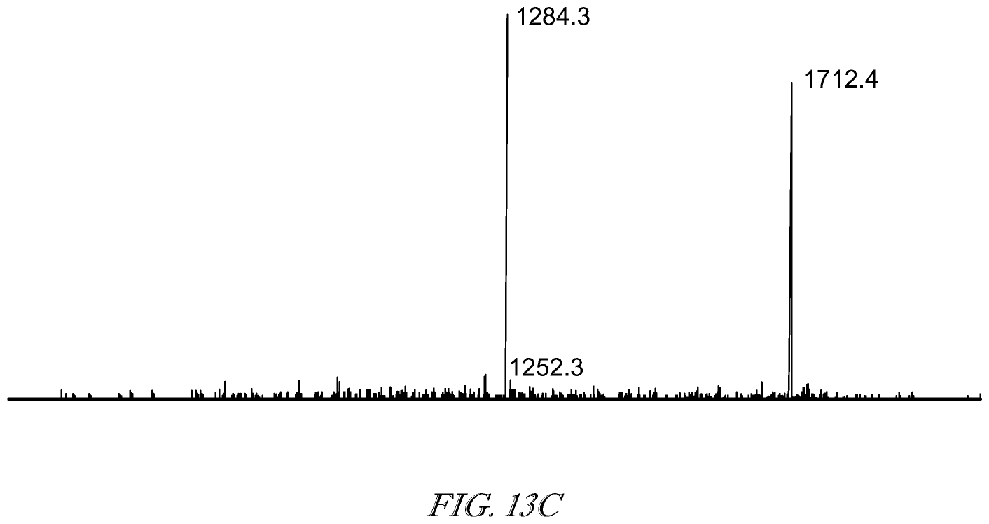

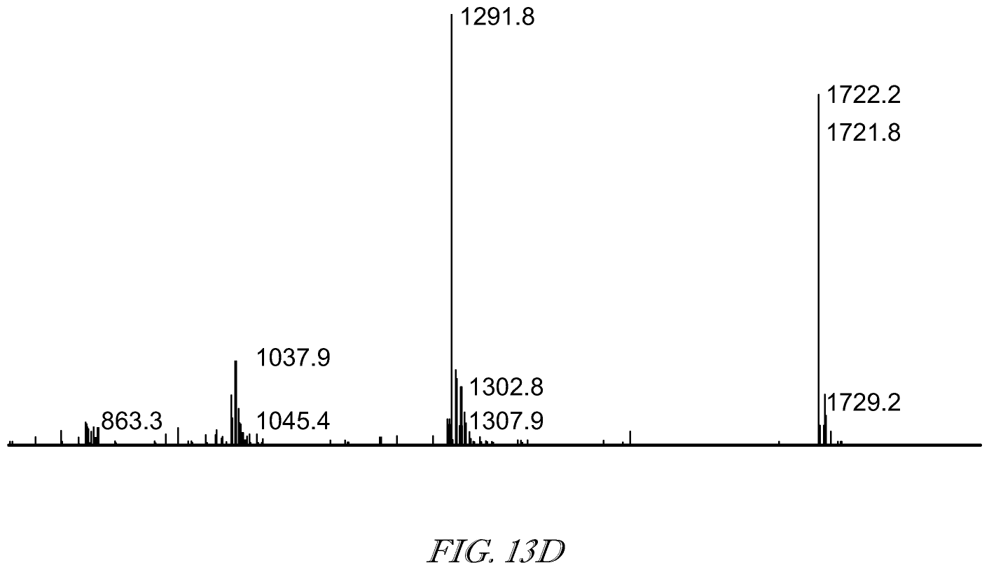

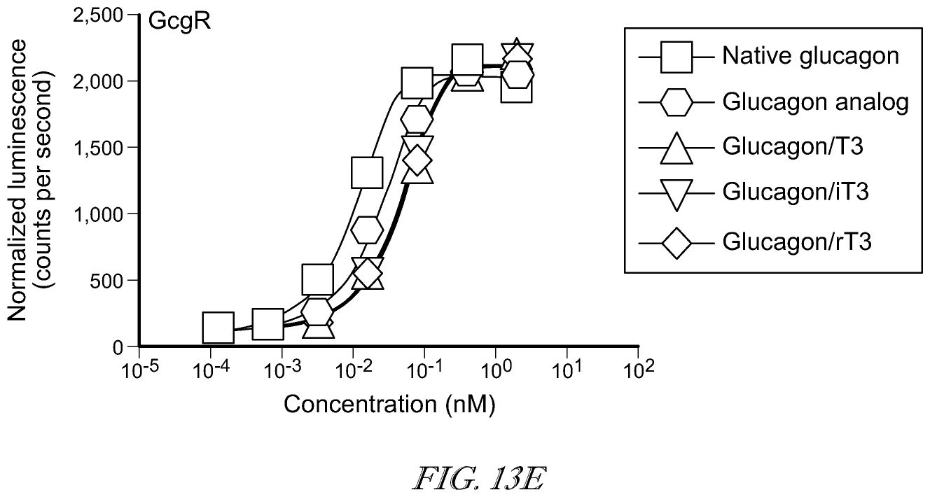

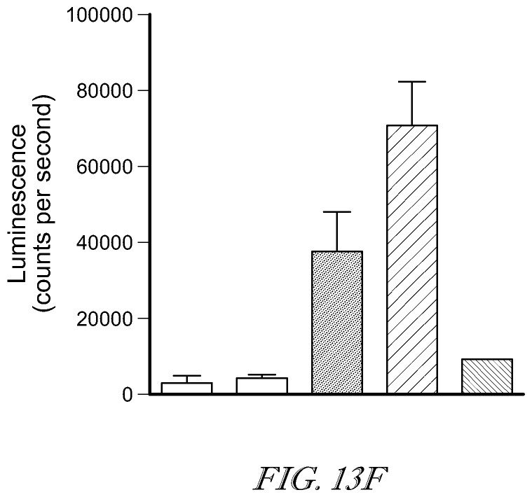

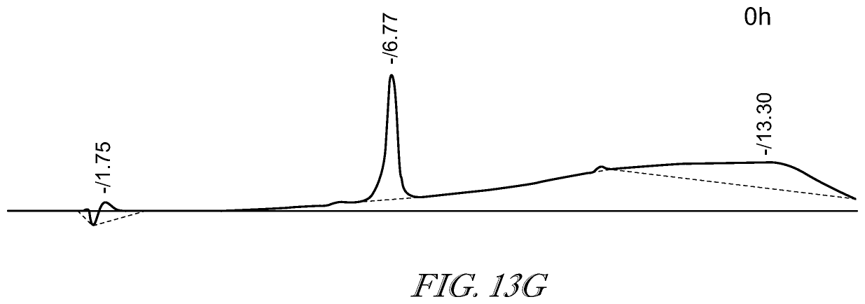

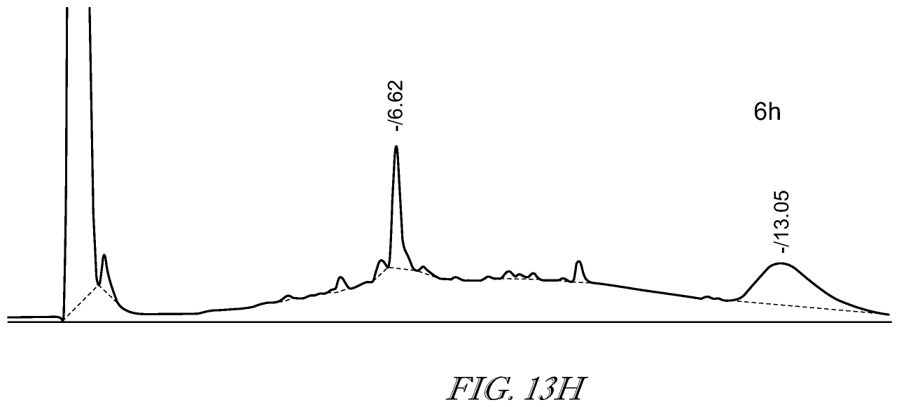

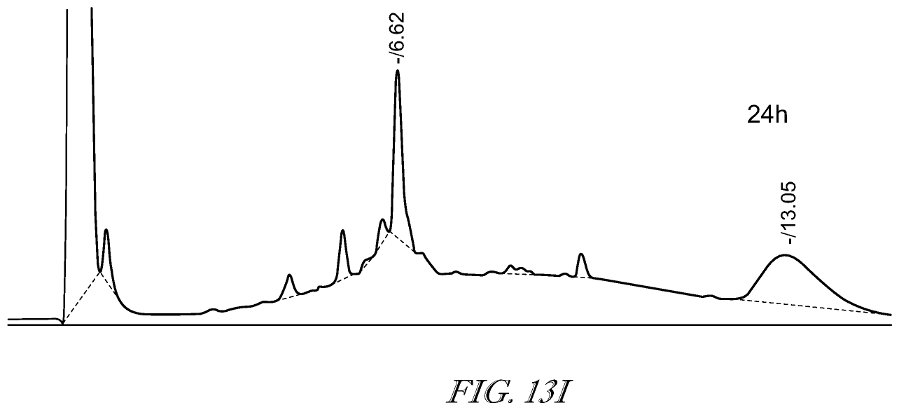

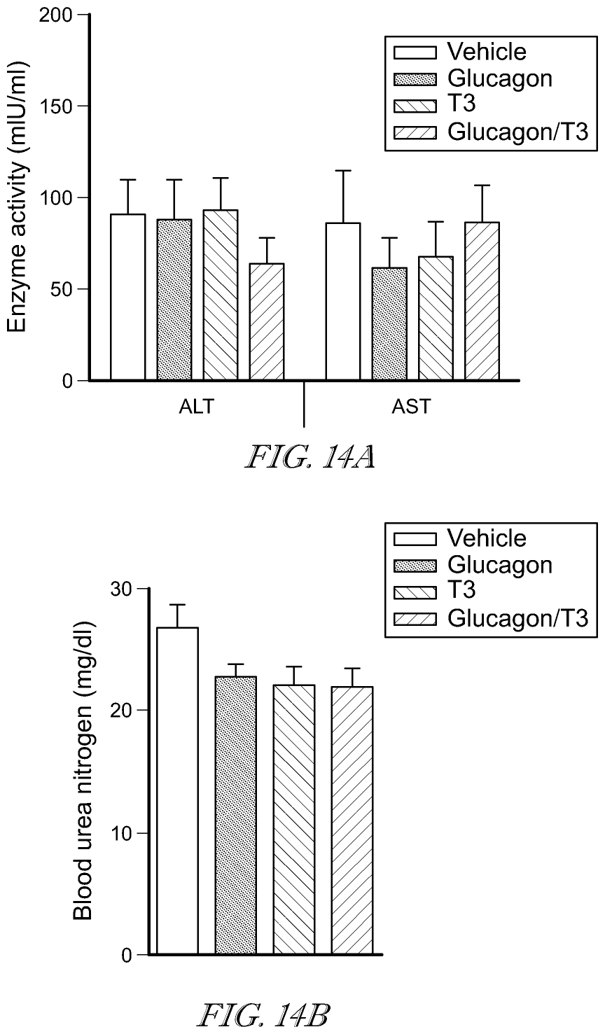

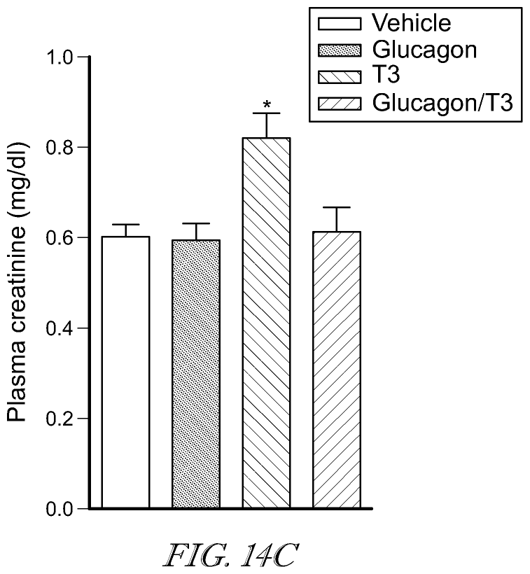

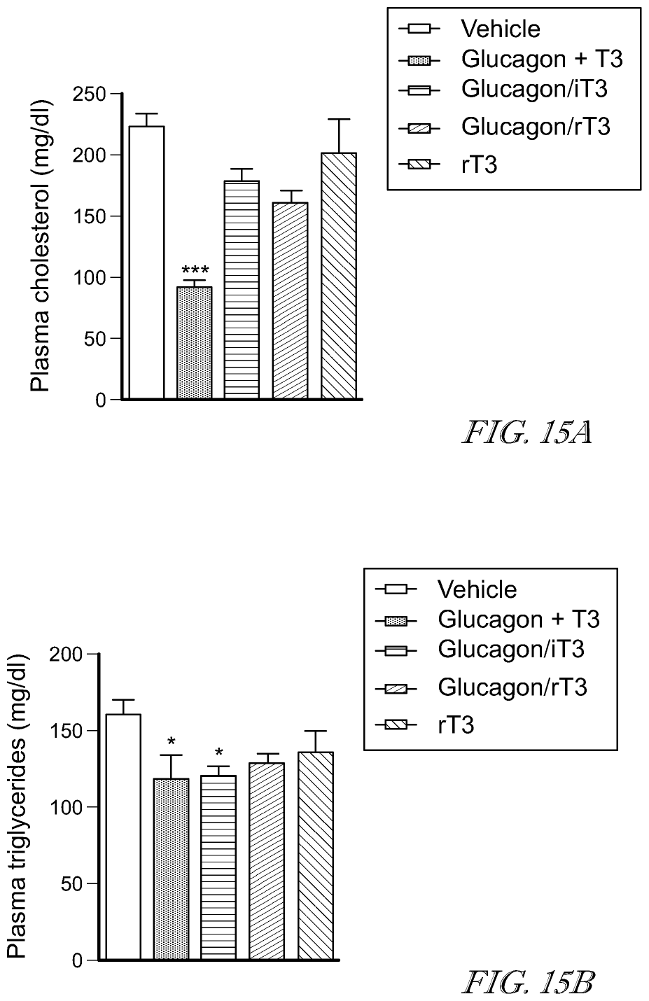

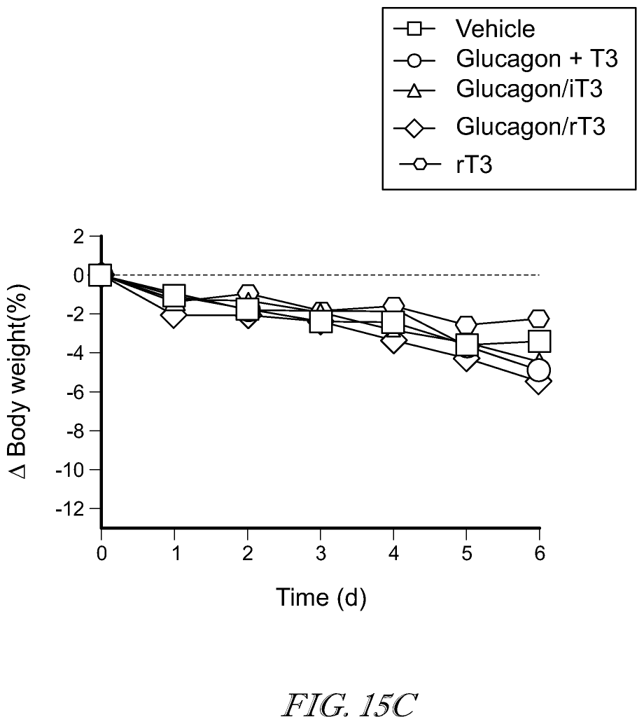

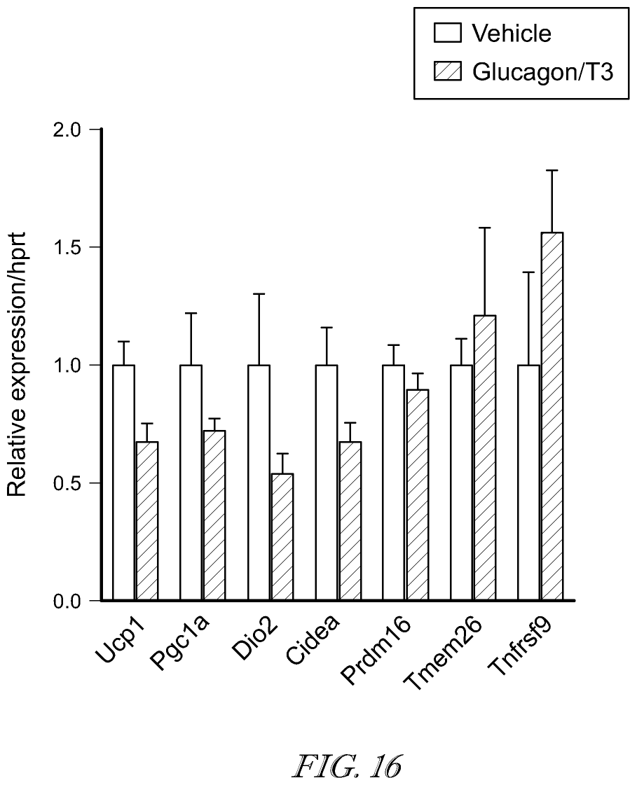

Effects on levels of T3 deposited in the liver (FIG. 1A), plasma levels of total cholesterol (FIG. 1B), cholesterol bound to different lipoprotein fractions (FIG. 1C), triglycerides (FIG. 1D; column 1, vehicle; column 2, glucagon; column 3, T3 and column 4, glucagon/T3), hepatic cholesterol (FIG. 1E; column 1, vehicle; column 2, glucagon; column 3, T3 and column 4, glucagon/T3), liver H & E staining and steatosis scoring (FIG. 1F), hepatic mRNA expression of select targets (FIG. 1G; solid bar, vehicle; open bar, glucagon; shades bar, T3 and cross hatched bar, glucagon/T3), and plasma levels of FGF21 from HFHCD fed male C57B16j mice (FIG. 1H) following daily subcutaneous injections of vehicle, a glucagon analog, T3, or glucagon/T3 at a dose of 100 nmol kg-1 for 14 days (n=8). Effects on plasma levels of total cholesterol (FIG. 1I), cholesterol bound to different lipoprotein fractions (FIG. 1J), and atherosclerotic plaque coverage expressed as the percentage of oil-red 0 positive area per total area in the aortic root (FIG. 1K) following daily subcutaneous injections of vehicle or glucagon/T3 at a dose of 100 nmol kg-1 for 14 days (n=10). *p<0.05, **p<0.01, and ***p<0.001 comparing effects following compound injections to vehicle injections. All data are presented as mean±SEM.

FIGS. 2A-2D. Lipid Improvements of Glucagon/T3 Require GcgR and THRβ

Effects on plasma levels of (FIG. 2A) total cholesterol and (FIG. 2B) triglycerides from HFHSD-fed global GcgR−/− male mice following daily subcutaneous injections of vehicle or glucagon/T3 at a dose of 100 nmol kg-1 for 7 days (n=7-9). Effects on plasma levels of (FIG. 2C) total cholesterol and (FIG. 2D) triglycerides from western diet-fed Alf-THRβ−/− male mice following daily subcutaneous injections of vehicle or glucagon/T3 at a dose of 100 nmol kg-1 for 7 days (n=5-7). *p<0.05, **p<0.01, and ***p<0.001 comparing effects following compound injections to vehicle injections within each genotype.
All data are presented as mean±SEM.

FIGS. 3A-3D. Unbiased Transcriptional Profiling of Livers from Treated Mice RNA-seq analysis of livers from HFHCD-fed C57BL/6j male mice (n=4) following 14 days of daily treatment with vehicle, a glucagon analog, T3, the equimolar co-administration of the glucagon analog and T3, and the glucagon/T3 conjugate. (FIG. 3A) Overlap of genes significantly regulated (>2-fold change) by the different treatment groups compared to vehicle controls. (FIG. 3B) Top pathways enriched in the liver by treatment with glucagon/T3 with associated −log 10 P values. Each dot displays one significant regulated gene/transcript mapped to the Pathway shown (yaxis). The log 2-FC is indicated by the x-axis. Size of the dots on the far right corresponds to the negative log 10(p-value) for the enrichment. (FIG. 3C) Comparison of the magnitude of the fold change in transcription between similar genes regulated by both T3 alone and glucagon/T3. (FIG. 3D) Magnitude of the fold change in transcription between targets that are regulated in the same direction by both the co-administration of glucagon and T3 compared to glucagon/T3. To detect for synergistic like effects, we calculated a synergy score (SS, see methods/results for details) for each expressed transcript and found 208 synergistic targets. For each target the log 2FC for treatment with glucagon+T3 co-administration and glucagon/T3 is shown.

FIG. 4A-4K. Glucagon/T3 Increases Energy Expenditure and Lowers Body Weight in DIO Mice

Effects on body weight change (FIG. 4A), body composition (FIG. 4B), cumulative food intake (FIG. 4C), longitudinal energy expenditure (FIG. 4D), cumulative locomotor activity (FIG. 4E), plasma levels of T3 (FIG. 4F), rectal temperature (FIG. 4G), and average RER (FIG. 4H) during the light and dark phase during a 24 hour period between days 2 and 3 of treatment from HFHSD-fed male C57B16j mice following daily subcutaneous injections of vehicle, a glucagon analog, T3, or glucagon/T3 at a dose of 100 nmol kg-1 for 7 days (n=8). Effects on body weight change (FIG. 4I), longitudinal energy expenditure (FIG. 4J), and average RER during the light and dark phase (FIG. 4K) during a 24 hour period between days 2 and 3 of treatment from HFHSD-fed global GcgR−/− male mice or wild-type controls following daily subcutaneous injections of vehicle or glucagon/T3 at a dose of 100 nmol kg-1 for 7 days (n=7-9). *p<0.05, **p<0.01, and ***p<0.001 comparing effects following compound injections to vehicle injections within comparable genotypes. All data are presented as mean±SEM.

FIGS. 5A-5F. Glucagon/T3 Induces Browning of iWAT and Full Weight-Lowering Efficacy Depends on UCP-1 Mediated Thermogenesis

Effects on levels of T3 deposited in iWAT (FIG. 5A), iWAT H & E staining (FIG. 5B), and iWAT mRNA expression of select targets (FIG. 5C), following daily subcutaneous injections of vehicle, a glucagon analog, T3, or glucagon/T3 at a dose of 100 nmol kg-1 for 14 days (n=8). Effects on body weight change (FIG. 5D), average RER during the light and dark phase (FIG. 5E) during a 24 hour period between days 2 and 3 of treatment, and longitudinal energy expenditure from HFHSD-fed global Ucp1−/− male mice (FIG. 5F) or wildtype controls maintained at 30° C. following daily subcutaneous injections of vehicle or glucagon/T3 at a dose of 100 nmol kg-1 for 7 days (n=4-7). *p<0.05, **p<0.01, and ***p<0.001 comparing effects following compound injections to vehicle injections. All data are presented as mean±SEM.

FIG. 6A-6I. The T3 Action of Glucagon/T3 Overpowers the Hyperglycemic Effects of Glucagon

Effects on fasted blood glucose through 120 min and ad libitum-fed blood glucose at 16 h from HFHSD-fed male C57B16j mice (FIG. 6A) following a single subcutaneous injection of vehicle, a glucagon analog, T3, or glucagon/T3 at a dose of 100 nmol kg-1 (n=8). Effects on intraperitoneal glucose tolerance (1.5 g kg-1) (FIG. 6B), intraperitoneal insulin tolerance (0.75 IU kg-1) (FIG. 6C), plasma levels of insulin (FIG. 6D), intraperitoneal pyruvate tolerance (1.5 g kg-1) at the indicated days from HFHSD-fed male C57B16j mice (FIG. 6E) following daily subcutaneous injections of vehicle, a glucagon analog, T3, or glucagon/T3 at a dose of 100 nmol kg-1 (n=8). Acute effects on RER during the light phase of the second day of treatment from HFHSD-fed male C57B16j mice (FIG. 6F) immediately following a subcutaneous injection of vehicle, a glucagon analog, T3, or glucagon/T3 at a dose of 100 nmol kg-1 (n=8). Effects on plasma levels of free fatty acids from HFHSD-fed male C57B16j mice (FIG. 6G) following a single subcutaneous injection of vehicle, a glucagon analog, T3, or glucagon/T3 at a dose of 100 nmol kg-1 (n=8). Effects on hepatic mRNA expression of select targets indicative of glucose metabolism (FIG. 6H) and the PGC-1 axis from HFHCD-fed male C57B16j mice (FIG. 6I) following daily subcutaneous injections of vehicle, a glucagon analog, T3, or glucagon/T3 at a dose of 100 nmol kg-1 for 14 days (n=8). *p<0.05, **p<0.01, and ***p<0.001 comparing effects following compound injections to vehicle injections. All data are presented as mean±SEM.

FIGS. 7A-7I. Glucagon/T3 is Devoid of Adverse Effects on Cardiac Function

Effects on heart rate (FIG. 7A), respiration rate (FIG. 7B), fraction shortening (FIG. 7C), ejection fraction (FIG. 7D), heart weight to tibia length ratio (FIG. 7E), left ventricular internal diameter at the end of diastole (FIG. 7F) and systole (FIG. 7G), and whole heart mRNA expression of T3-sensitive targets (FIG. 7H) and surrogate hypertrophic markers (FIG. 7I) from HFHSD-fed male C57B16j mice following daily subcutaneous injections of vehicle, a glucagon analog, T3, or glucagon/T3 at a dose of 100 nmol kg-1 (n=8) for 28 days. *p<0.05, **p<0.01, and ***p<0.001 comparing effects following compound injections to vehicle injections. All data are presented as mean±SEM. For each of the graphs, the solid bars represent vehicle; open bars represent glucagon; stippled bars represent T3 and cross hatched bars represent glucagon/T3.

FIG. 8 Presents the metabolic pathways for Thyroxine (T4).

FIG. 9 Presents the chemical structures of Triiodothyronine (T3) and various known analogs thereof.

FIG. 10 Presents the chemical structures of L-thyroxine and its enantiomer Dextrothyroxine which was used in an early clinical trial to treat dyslipidemia; as well as the chemical structures of various thyroxine analogs including the organ-selective analogs L-94901 and T-0681, and TRβ1-selective analogs GC-1, CGS23425, KB-141, DITPA, and MB07344, the active form of the prodrug MB07811.

FIG. 11 Presents the chemical structures of Triiodothyronine (T3) and various known analogs thereof.

FIGS. 12A-12E. Chemical Structures of Glucagon and T3 Conjugates.

Sequence, structure, molecular weight and GcgR activity of native glucagon (FIG. 12A; SEQ ID NO: 1), the glucagon analog used for creation of conjugates (FIG. 12B; SEQ ID NO: 934), glucagon/T3 (FIG. 12C; glucagon sequence=SEQ ID NO: 934), glucagon/iT3 (FIG. 12D; glucagon sequence=SEQ ID NO: 934), and glucagon/rT3 (FIG. 12E; glucagon sequence=SEQ ID NO: 934).

FIGS. 13A-13I. In Vitro Profiling of Glucagon/T3 Character, Constituent Receptor Activity, and Stability. Mass spectrometry confirming the identity of the glucagon analog (FIG. 13A), glucagon/T3 (FIG. 13B), glucagon/iT3 (FIG. 13C), and glucagon/rT3 (FIG. 13D). Receptor activity profiles of the conjugates at GcgR (FIG. 13E) and THR (FIG. 13F) using DR4-luciferase reporter assays. HPLC chromatograms of glucagon/T3 incubated in human plasma at 37° C. after 0 h (FIG. 13G), 6 h (FIG. 13H), and 24 h (FIG. 13I) exposure.

FIGS. 14A-14C. Glucagon/T3 Does Not Harm Tissue Function or Thyroid Hormone Endocrinology. Effects on plasma levels of ALT and AST (FIG. 14A), blood urea nitrogen (FIG. 14B) and creatinine from HFHCD-fed male C57B16j mice (FIG. 14C) following daily subcutaneous injections of vehicle, a glucagon analog, T3, or glucagon/T3 at a dose of 100 nmol kg-1 for 14 days (n=8).

FIGS. 15A-15C. Metabolic Efficacy of Different Glucagon/T3 Conjugate Versions. Effects on plasma levels of total cholesterol (FIG. 15A), triglycerides (FIG. 15B), and percent body weight loss from HFHSD-fed male C57B16j mice (15C) following daily subcutaneous injections of vehicle, equimolar co-administration of the glucagon analog and T3, glucagon/iT3, glucagon/rT3, or rT3 at a dose of 100 nmol kg-1 for 14 days (n=8). *p<0.05 and ***p<0.001 comparing effects following compound injections to vehicle injections. All data are presented as mean±SEM.

FIG. 16. Thermogenic gene program in classical BAT. Effects on the relative expression of selected theromegenic genes in BAT from HFHCD-fed male C57B16j mice following daily subcutaneous injections of vehicle or glucagon/T3 at a dose of 100 nmol kg.

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. For the purposes of the present disclosure designation of an amino acid without specifying its stereochemistry is intended to encompass either the L or D form of the amino acid, or a racemic mixture. The D isomer of native amino acids is indicated by a lower case “d” preceding the standard 3 letter amino acid code (e.g., dSer).

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “bioactive polypeptide” refers to polypeptides which are capable of exerting a biological effect in vitro and/or in vivo.

As used herein a general reference to a peptide is intended to encompass peptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, and (v) increased potency at the IGF and/or insulin peptide receptors.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases and increased potency at the IGF and/or insulin receptors.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “hydrophilic moiety” refers to any compound that is readily water-soluble or readily absorbs water, and which are tolerated in vivo by mammalian species without toxic effects (i.e. are biocompatible). Examples of hydrophilic moieties include polyethylene glycol (PEG), polylactic acid, polyglycolic acid, a polylactic-polyglycolic acid copolymer, polyvinyl alcohol, polyvinylpyrrolidone, polymethoxazoline, polyethyloxazoline, polyhydroxyethyl methacrylate, polyhydroxypropyl methacrylamide, polymethacrylamide, polydimethylacrylamide, and derivatised celluloses such as hydroxymethylcellulose or hydroxyethylcellulose and co-polymers thereof, as well as natural polymers including, for example, albumin, heparin and dextran.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of an glucagon analog refers to a nontoxic but sufficient amount of a glucagon analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “derivative” is intended to encompass chemical modification to a compound (e.g., an amino acid), including chemical modification in vitro, e.g. by introducing a group in a side chain in one or more positions of a polypeptide, e.g. a nitro group in a tyrosine residue, or iodine in a tyrosine residue, or by conversion of a free carboxylic group to an ester group or to an amide group, or by converting an amino group to an amide by acylation, or by acylating a hydroxy group rendering an ester, or by alkylation of a primary amine rendering a secondary amine or linkage of a hydrophilic moiety to an amino acid side chain. Other derivatives are obtained by oxidation or reduction of the side-chains of the amino acid residues in the polypeptide.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

The term “glucagon agonist peptide” refers to a compound that binds to and activates downstream signaling of the glucagon receptor.

As used herein, “thyroid hormone receptor ligand” refers to a compound that has biological agonist activity and binds to and activates downstream signaling of the thyroid hormone receptor. The thyroid hormone receptor ligand is wholly or partly non-peptidic.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

Throughout the application, all references to a particular amino acid position by number (e.g., position 28) refer to the amino acid at that position in native glucagon (SEQ ID NO: 1) or the corresponding amino acid position in any analogs thereof. For example, a reference herein to “position 28” would mean the corresponding position 27 for an analog of glucagon in which the first amino acid of SEQ ID NO: 1 has been deleted. Similarly, a reference herein to “position 28” would mean the corresponding position 29 for an analog of glucagon in which one amino acid has been added before the N-terminus of SEQ ID NO: 1. In addition a reference to a position greater than 29 (native glucagon only has 29 amino acids) is intended to refer to amino acid position in an analog having a C-terminus amino acid extension after the corresponding position 29 of SEQ ID NO: 1

As used herein the general term “polyethylene glycol chain” or “PEG chain”, refers to mixtures of condensation polymers of ethylene oxide and water, in a branched or straight chain, represented by the general formula H(OCH₂CH₂)—OH, wherein n is at least 2. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-Butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like.

The term “C₂-Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃alkyl)(C₆-C₁₀ aryl)” refers to a 5 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆heteroaryl)” refers to a 5 or 6 membered heteroaryl that is attached to a parent moiety via a one to “n” membered alkyl chain.

As used herein, the term “halo” refers to one or more members of the group consisting of fluorine, chlorine, bromine, and iodine.

As used herein the term “patient” without further designation is intended to encompass any warm blooded vertebrate domesticated animal (including for example, but not limited to livestock, horses, cats, dogs and other pets) and humans.

The term “isolated” as used herein means having been removed from its natural environment. In some embodiments, the analog is made through recombinant methods and the analog is isolated from the host cell.

The term “purified,” as used herein relates to the isolation of a molecule or compound in a form that is substantially free of contaminants normally associated with the molecule or compound in a native or natural environment and means having been increased in purity as a result of being separated from other components of the original composition. The term “purified polypeptide” is used herein to describe a polypeptide which has been separated from other compounds including, but not limited to nucleic acid molecules, lipids and carbohydrates.

As used herein, the term “peptide” encompasses a sequence of 2 or more amino acids and typically less than 50 amino acids, wherein the amino acids are naturally occurring or coded or non-naturally occurring or non-coded amino acids. Non-naturally occurring amino acids refer to amino acids that do not naturally occur in vivo but which, nevertheless, can be incorporated into the peptide structures described herein. “Non-coded” as used herein refer to an amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

As used herein the term “hydroxyl acid” refers to an amino acid that has been modified to replace the alpha carbon amino group with a hydroxyl group.

As used herein, “partly non-peptidic” refers to a molecule wherein a portion of the molecule is a chemical compound or substituent that has biological activity and that does not comprises a sequence of amino acids.

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. For example a peptidomimetic may include a sequence of naturally-occurring amino acids with the insertion or substitution of a non-peptide moiety, e.g. a retroinverso fragment, or incorporation of non-peptide bonds such as an azapeptide bond (CO substituted by NH) or pseudo-peptide bond (e.g. NH substituted with CH₂), or an ester bond (e.g., depsipeptides, wherein one or more of the amide (—CONHR—) bonds are replaced by ester (COOR) bonds). Alternatively the peptidomimetic may be devoid of any naturally-occurring amino acids.

As used herein the term “charged amino acid” or “charged residue” refers to an amino acid that comprises a side chain that is negatively charged (i.e., de-protonated) or positively charged (i.e., protonated) in aqueous solution at physiological pH. For example, negatively charged amino acids include aspartic acid, glutamic acid, cysteic acid, homocysteic acid, and homoglutamic acid, whereas positively charged amino acids include arginine, lysine and histidine. Charged amino acids include the charged amino acids among the 20 amino acids commonly found in human proteins, as well as atypical or non-naturally occurring amino acids.

As used herein the term “acidic amino acid” refers to an amino acid that comprises a second acidic moiety (other than the alpha carboxylic acid of the amino acid), including for example, a side chain carboxylic acid or sulfonic acid group.

As used herein, the term “prodrug” is defined as any compound that undergoes chemical modification before exhibiting its full pharmacological effects.

As used herein, a “dipeptide” is the result of the linkage of an α-amino acid or α-hydroxyl acid to another amino acid, through a peptide bond.

As used herein the term “chemical cleavage” absent any further designation encompasses a non-enzymatic reaction that results in the breakage of a covalent chemical bond.

As used herein the term “glucagon/T3 conjugates” is a generic reference to any conjugate that comprises a peptide having the ability to bind and activate the glucagon receptor and a second compound having the ability to bind and activate the thyroid hormone receptor.

### EMBODIMENTS

Thyroid hormones have profound effects on lipid, cholesterol and glucose metabolism through liver-specific actions. Thyroid hormones also have substantial effects on thermogenesis and lipolysis through adipose-specific actions. These combined actions make thyroid hormone an attractive drug candidate for the treatment of dyslipidemia and obesity. However, adverse effects primarily in the cardiovascular system have until now precluded its use for chronic treatment of metabolic diseases. Importantly, the beneficial functions of thyroid hormone on systemic metabolism are largely aligned with chronic actions of glucagon on lipid metabolism and body weight. As disclosed herein by using glucagon as a targeting ligand, unbiased thyroid hormone action can be selectively guided to the liver and adipose depots, where synergistic benefits on lipid metabolism and adiposity are unleashed. Importantly, the disclosed conjugates uncouple the metabolic benefits from deleterious effects on the cardiovascular system that would otherwise arise from systemic thyroid hormone action. Furthermore, the liver-specific effects of thyroid hormone action counteract the diabetogenic effects of glucagon action, completing mutual cancellation of the inherent limitations of each hormone. Unimolecular integration of thyroid hormone and glucagon action profiles synergize to maximize comprehensive metabolic benefits while masking their harmful effects that had prevented their individual use.

Applicants disclose herein compositions and methods for glucagon-mediated selective delivery of thyroid hormone action to the liver as a primary target and to inguinal white fat (iWAT) as a secondary target. Together, coordinated glucagon and thyroid hormone actions synergize to correct hyperlipidemia, reverse hepatic steatosis and lower body weight through liver and fat-specific mechanisms.

Provided herein are chemical conjugates of a glucagon agonist peptide and compounds having thyroid hormone activity (“glucagon/T3 conjugates”). These conjugates with plural activities are useful for the treatment of a variety of diseases including hyperlipidemia, metabolic syndrome, diabetes, obesity, liver steatosis, and chronic cardiovascular disease.

As disclosed herein chemical conjugates of glucagon and thyroid hormone (glucagon/T3) have been engineered to capitalize on the preferential sites of glucagon action to precisely harness T3 action in select tissues. Coordinated glucagon and T3 actions synergize to correct hyperlipidemia, hepatic steatosis, atherosclerosis, glucose intolerance and obesity in patients. Each hormonal constituent of the conjugate retains its native activity and mutually enriches cellular processes in hepatocytes and adipocytes. Synchronized signaling driven by glucagon and T3 reciprocally minimizes the inherent harmful effects of each hormone. Liver directed T3 action offsets the diabetogenic liability of glucagon, and glucagon-mediated delivery spares the cardiovascular system from adverse T3 action.

The glucagon agonist peptide conjugates of the present disclosure can be represented by the following formula:

Q-L-Y

wherein Q is a glucagon agonist peptide, Y is a thyroid hormone receptor ligand, and L is a linking group or a bond joining Q to Y.

Compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

As disclosed herein glucagon and T3 signaling pathways converge to reverse hypercholesterolemia through pleotropic mechanisms. The observed synergism and reciprocal regulation of certain gene targets offer clues to molecular underpinnings that could be mediating many of the responses observed on hepatic cholesterol handling by a single molecule glucagon/T3 conjugate.

Lipid deposition in the liver is a key factor in hepatic insulin resistance and the pathogenesis of type 2 diabetes. Hepatic steatosis is a predisposing determinant in liver diseases not commonly associated with diabetes, including nonalcoholic steatohepatitis (NASH), cirrhosis, and hepatocellular carcinomas. Glucagon and thyroid hormone have individually been shown to have beneficial effects on hepatic triglyceride metabolism. As demonstrated herein glucagon-mediated targeting of T3 effectively removes fat deposition in the liver more potently than either agent alone without worsening insulin sensitivity or promoting hyperthermia. The secondary effects of glucagon/T3 on adipose tissue and the associated modest weight-lowering efficacy will only further support alleviating disease symptoms of NASH.

Accordingly in one embodiment a method is provided for alleviating disease symptoms of NASH, wherein the method comprises administering a glucagon/T3 conjugate to a patient in need thereof. The glucagon/T3 conjugate acts as a specific pharmacological agent targeted to the liver in order to directly counteract the localized gluconeogenesis and glycogenolysis induced by glucagon. The addition of thyroid hormone action lessens the acute rise in blood glucose that is otherwise seen with unopposed glucagon administration, and improves glucose utilization after glucose, insulin, and pyruvate challenges that are ordinarily deteriorated after chronic glucagon treatment.

In accordance with one embodiment a method of inducing weight loss or preventing weight gain is provided, wherein the method comprises administering a glucagon/T3 conjugate to a patient in need thereof. The weight loss following glucagon/T3 therapy is due to increased energy expenditure, some of which is mediated via lipolytic mechanisms and the recruitment of thermogenesic-capable adipocytes in iWAT. The primary mechanism responsible for non-shivering thermogenesis in adipocytes is coordinated lipolysis and concurrent uncoupling of the mitochondrial respiratory chain via UCP1 to allow for rapid fatty acid oxidation, minimal ATP production, and heat production. Both glucagon and T3 have individually been reported to increase UCP1 activity in vivo.

Glucagon/T3 conjugates causes mobilization and utilization of triglycerides and cholesterol, and prevents the accumulation of atherosclerotic plaques in the aortic root, all of which are vital to reduce CHD risk. In accordance with one embodiment a method is provided for reducing the accumulation of atherosclerotic plaques in a patient and treat chronic cardiovascular disease, wherein the method comprises administering a glucagon/T3 conjugate to a patient in need thereof. In one embodiment a glucagon/T3 conjugate is administered to a patient to decrease low-density lipoprotein, triglycerides, apolipoprotein B, and lipoprotein(a) levels.

Importantly, the synergistic effects of glucagon and T3 co-agonism translate to less reliance on individual signaling cues to have equal potency as the single hormones. Thus lower circulating concentrations of the conjugate are needed to elicit lipid lowering and body weight-lowering effects, which presumably contribute to the enhanced safety profile.

### Thyroid Hormone Receptor Ligand Agonists

Thyroxine (T₄) is a thyroid hormone involved in the control of cellular metabolism. Chemically, thyroxine is an iodinated derivative of the amino acid tyrosine. The maintenance of a normal level of thyroxine is important for normal growth and development of children as well as for proper bodily function in the adult. Its absence leads to delayed or arrested development. Hypothyroidism, a condition in which the thyroid gland fails to produce enough thyroxine, leads to a decrease in the general metabolism of all cells, most characteristically measured as a decrease in nucleic acid and protein synthesis, and a slowing down of all major metabolic processes. Conversely, hyperthyroidism is an imbalance of metabolism caused by overproduction of thyroxine.

During metabolism, T4 is converted to T3 or to rT3 via removal of an iodine atom from one of the hormonal rings. T3 is the biologically active thyroid hormone, whereas rT3 has no biological activity. Both T3 and T4 are used to treat thyroid hormone deficiency (hypothyroidism). They are both absorbed well by the gut, so can be given orally.

In accordance with the present disclosure a conjugate is provided comprising a thyroid receptor ligand that is covalently linked to a glucagon agonist peptide. More particularly in one embodiment the thyroid receptor ligand (Y) of the Q-L-Y conjugate, is thyroid hormone or a thyroid hormone receptor agonist that binds and activates the thyroid receptor. Suitable compounds include any of the compounds disclosed in FIGS. 8-11 or a compound having the general structure of

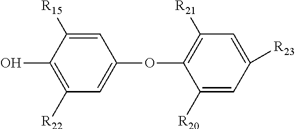

wherein

R₁₅ is C₁-C₄ alkyl, —CH₂(pyridazinone), —CH₂(OH)(phenyl)F, —CH(OH)CH₃, halo or H;

R₂₀ is halo, CH₃ or H;

R₂₁ is halo, CH₃ or H;

R₂₂ is H, OH, halo, —CH₂(OH)(C₆ aryl)F, or C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, or —OCH₂PO₃²⁻.

In accordance with one embodiment the thyroid hormone component (Y) is a compound of the general structure

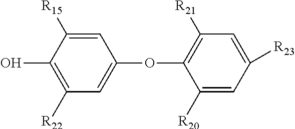

wherein

R₁₅ is C₁-C₄ alkyl, —CH(OH)CH₃, I or H

R₂₀ is I, Br, CH₃ or H;

R₂₁ is I, Br, CH₃ or H;

R₂₂ is H, OH, I, or C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, or —OCH₂PO₃²⁻. In one embodiment R₂₃ is —CH₂CH(NH₂)COOH.

In accordance with one embodiment the thyroid hormone component (Y) is a compound of the general structure

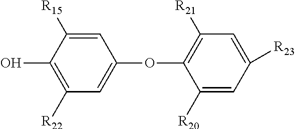

wherein

R₁₅ is isopropyl, —CH(OH)CH₃, I or H

R₂₀ is I, Br, Cl, or CH₃;

R₂₁ is I, Br, Cl, or CH₃;

R₂₂ is H; and

R₂₃ is —OCH₂COOH, —CH₂COOH, —NHC(O)CH₂COOH, or —CH₂CH₂COOH.

In accordance with one embodiment the thyroid hormone component (Y) is a compound of the general structure of Formula I:

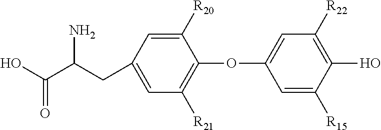

wherein

R₂₀, R₂₁, and R₂₂ are independently selected from the group consisting of H, OH, halo and C₁-C₄ alkyl; and

R₁₅ is halo or H. In one embodiment R₂₀ and R₂₁ are each CH₃, R₁₅ is H and R₂₂ are independently selected from the group consisting of H, OH, halo and C₁-C₄ alkyl. In one embodiment R₂₀, R₂₁ and R₂₂ are each halo and R₁₅ is H or halo. In a further embodiment R₂₀, R₂₁ and R₂₂ are each I or Cl, and R₁₅ is H or I. In a further embodiment R₂₀, R₂₁ and R₂₂ are each I, and R₁₅ is H or I.

In accordance with one embodiment Y is selected from the group consisting of thyroxine T4 (3,5,3′,5′-tetraiodothyronine) and 3,5,3′-triiodo L-thyronine.

In one embodiment, the thyroid receptor ligand (Y) of the Q-L-Y conjugates, is an indole derivative of thyroxine, including for example, compounds disclosed in U.S. Pat. No. 6,794,406 and US published application no. US 2009/0233979, the disclosures of which are incorporated herein. In one embodiment the indole derivative of thyroxine comprises a compound of the general structure of Formula II:

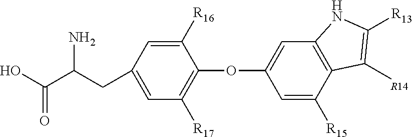

wherein

R₁₃ is H or C₁-C₄ alkyl;

R₁₄ is C₁-C₈ alkyl;

R₁₅ is H or C₁-C₄ alkyl; and

R₁₆ and R₁₇ are independently halo or C₁-C₄ alkyl.

In one embodiment, the thyroid receptor ligand (Y) of the Q-L-Y conjugates, is an indole derivative of thyroxine as disclosed in WO97/21993 (U. Cal SF), WO99/00353 (KaroBio), GB98/284425 (KaroBio), and U.S. Provisional Application 60/183,223, the disclosures of which are incorporated by reference herein. In one embodiment the thyroid receptor ligand comprises the general structure of Formula III:

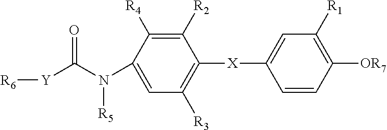

wherein X is oxygen, sulfur, carbonyl, methylene, or NH;

Y is (CH₂)ₙ where n is an integer from 1 to 5, or C═C;

R₁ is halogen, trifluoromethyl, or C₁-C₆ alkyl or C₃-C₇ cycloalkyl;

R₂ and R₃ are the same or different and are hydrogen, halogen, C₁-C₆ alkyl or C₃-C₇ cycloalkyl, with the proviso that at least one of R₂ and R₃ being other than hydrogen;

R₄ is hydrogen or C₁-C₄ alkyl;

R₅ is hydrogen or C₁-C₄ alkyl;

R₆ is carboxylic acid, or ester thereof; and

R₇ is hydrogen, or an alkanoyl or aroyl group.

### The Glucagon Agonist Peptide (Q)

In one embodiment, Q of the Q-L-Y conjugates described herein is a native glucagon peptide comprising the sequence of SEQ ID NO: 1. In one embodiment Q is glucagon agonist peptide wherein the native sequence of glucagon has up to 10 modifications relative to the native sequence. A glucagon agonist peptide refers to a group of peptides related in structure in their N-terminal and/or C-terminal regions (see, for example, Sherwood et al., Endocrine Reviews 21: 619-670 (2000)). It is believed that the C-terminus generally functions in receptor binding and the N-terminus generally functions in receptor signaling. A few amino acids in the N-terminal and C-terminal regions are highly conserved among members of the glucagon agonist. Some of these conserved amino acids include Gly4, Phe6, Phe22, Val23, Trp25 and Leu26, with amino acids at these positions showing identity, conservative substitutions or similarity in the structure of their amino acid side chains.

In some embodiments, Q exhibits an EC₅₀ for glucagon receptor activation (or an IC₅₀ for glucagon receptor antagonism) of about 10 mM or less, or about 1 mM (1000 μM) or less (e.g., about 750 μM or less, about 500 μM or less, about 250 μM or less, about 100 μM or less, about 75 μM or less, about 50 μM or less, about 25 μM or less, about 10 μM or less, about 7.5 μM or less, about 6 μM or less, about 5 μM or less, about 4 μM or less, about 3 μM or less, about 2 μM or less or about 1 μM or less). In some embodiments, Q exhibits an EC₅₀ or IC₅₀ at the glucagon receptor of about 1000 nM or less (e.g., about 750 nM or less, about 500 nM or less, about 250 nM or less, about 100 nM or less, about 75 nM or less, about 50 nM or less, about 25 nM or less, about 10 nM or less, about 7.5 nM or less, about 6 nM or less, about 5 nM or less, about 4 nM or less, about 3 nM or less, about 2 nM or less or about 1 nM or less). In some embodiments, Q has an EC₅₀ or IC₅₀ at the glucagon receptor which is in the picomolar range. Accordingly, in some embodiments, Q exhibits an EC₅₀ or IC₅₀ at the glucagon receptor of about 1000 pM or less (e.g., about 750 pM or less, about 500 pM or less, about 250 pM or less, about 100 pM or less, about 75 pM or less, about 50 pM or less, about 25 pM or less, about 10 pM or less, about 7.5 pM or less, about 6 pM or less, about 5 pM or less, about 4 pM or less, about 3 pM or less, about 2 pM or less or about 1 pM or less).

In some embodiments, Q exhibits an EC₅₀ or IC₅₀ at the glucagon receptor that is about 0.001 pM or more, about 0.01 pM or more, or about 0.1 pM or more. Glucagon receptor activation (glucagon receptor activity) can be measured by in vitro assays measuring cAMP induction in HEK293 cells over-expressing the glucagon receptor, e.g., assaying HEK293 cells co-transfected with DNA encoding the glucagon receptor and a luciferase gene linked to cAMP responsive element as described in Example 2.

In some embodiments, Q exhibits about 0.001% or more, about 0.01% or more, about 0.1% or more, about 0.5% or more, about 1% or more, about 5% or more, about 10% or more, about 20% or more, about 30% or more, about 40% or more, about 50% or more, about 60% or more, about 75% or more, about 100% or more, about 125% or more, about 150% or more, about 175% or more, about 200% or more, about 250% or more, about 300% or more, about 350% or more, about 400% or more, about 450% or more, or about 500% or higher activity at the glucagon receptor relative to native glucagon (glucagon potency). In some embodiments, Q exhibits about 5000% or less or about 10,000% or less activity at the glucagon receptor relative to native glucagon. The activity of Q at a receptor relative to a native ligand of the receptor is calculated as the inverse ratio of EC₅₀s for Q versus the native ligand.

In one embodiment the native glucagon sequence is modified as follows:

### Improved Solubility

Native glucagon exhibits poor solubility in aqueous solution, particularly at physiological pH, with a tendency to aggregate and precipitate over time. In contrast, the glucagon agonist peptides in some embodiments exhibit at least 2-fold, 5-fold, or even higher solubility compared to native glucagon at a pH between 6 and 8, or between 6 and 9, for example, at pH 7 after 24 hours at 25° C.

Accordingly, in some embodiments, a glucagon agonist peptide has been modified relative to the wild type peptide of His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr (SEQ ID NO: 1) to improve the peptide's solubility in aqueous solutions, particularly at a pH ranging from about 5.5 to about 8.0, while retaining the native peptide's biological activity.

For example, the solubility of any of the glucagon agonist peptides described herein can be further improved by attaching a hydrophilic moiety to the peptide. Introduction of such groups also increases duration of action, e.g. as measured by a prolonged half-life in circulation.

In some embodiments, solubility is improved by adding charge to the glucagon agonist peptide by the substitution of native non-charged amino acids with charged amino acids selected from the group consisting of lysine, arginine, histidine, aspartic acid and glutamic acid, or by the addition of charged amino acids to the amino or carboxy terminus of the peptide.

In accordance with some embodiments, the glucagon agonist peptide has improved solubility due to the fact that the peptide is modified by amino acid substitutions and/or additions that introduce a charged amino acid into the C-terminal portion of the peptide, and in some embodiments at a position C-terminal to position 27 of SEQ ID NO: 1. Optionally, one, two or three charged amino acids may be introduced within the C-terminal portion, and in some embodiments C-terminal to position 27. In accordance with some embodiments, the native amino acid(s) at positions 28 and/or 29 are substituted with a charged amino acid, and/or one to three charged amino acids are added to the C-terminus of the peptide, e.g. after position 27, 28 or 29. In exemplary embodiments, one, two, three or all of the charged amino acids are negatively charged. In other embodiments, one, two, three or all of the charged amino acids are positively charged.

In specific exemplary embodiments, the glucagon agonist peptide may comprise any one or two of the following modifications: substitution of N28 with E; substitution of N28 with D; substitution of T29 with D; substitution of T29 with E; insertion of E after position 27, 28 or 29; insertion of D after position 27, 28 or 29. For example, D28E29, E28E29, E29E30, E28E30, D28E30.

In accordance with one exemplary embodiment, the glucagon agonist peptide comprises an amino acid sequence of SEQ ID NO: 811, or an analog thereof that contains 1 to 3 further amino acid modifications (described herein in reference to glucagon agonists) relative to native glucagon, or a glucagon agonist analog thereof. SEQ ID NO: 811 represents a modified glucagon agonist peptide, wherein the asparagine residue at position 28 of the native protein has been substituted with an aspartic acid. In another exemplary embodiment the glucagon agonist peptide comprises an amino acid sequence of SEQ ID NO: 838, wherein the asparagine residue at position 28 of the native protein has been substituted with glutamic acid. Other exemplary embodiments include glucagon agonist peptides of SEQ ID NOs: 824, 825, 826, 833, 835, 836 and 837.

Substituting the normally occurring amino acid at position 28 and/or 29 with charged amino acids, and/or the addition of one to two charged amino acids at the carboxy terminus of the glucagon agonist peptide, enhances the solubility and stability of the glucagon peptides in aqueous solutions at physiologically relevant pHs (i.e., a pH of about 6.5 to about 7.5) by at least 5-fold and by as much as 30-fold. Accordingly, glucagon agonist peptides of some embodiments retain glucagon activity and exhibit at least 2-fold, 5-fold, 10-fold, 15-fold, 25-fold, 30-fold or greater solubility relative to native glucagon at a given pH between about 5.5 and 8, e.g., pH 7, when measured after 24 hours at 25° C.

Additional modifications, e.g. conservative substitutions, which modifications are further described herein, may be made to the glucagon agonist peptide that still allow it to retain glucagon activity.

### Improved Stability

Any of the glucagon agonist peptides may additionally exhibit improved stability and/or reduced degradation, for example, retaining at least 95% of the original peptide after 24 hours at 25° C. Any of the glucagon agonist peptides disclosed herein may additionally exhibit improved stability at a pH within the range of 5.5 to 8, for example, retaining at least 75%, 80%, 90%, 95%, 96%, 97%, 98% or 99% of the original peptide after 24 hours at 25° C. In some embodiments, the glucagon agonist peptides of the invention exhibit improved stability, such that at least 75% (e.g., at least 80%, at least 85%, at least 90%, at least 95%, more than 95%, up to 100%) of a concentration of the peptide or less than about 25% (e.g., less than 20%, less than 15%, less than 10%, less than 5%, 4%, 3%, 2%, 1%, down to 0%) of degraded peptide is detectable at 280 nm by an ultraviolet (UV) detector after about 1 or more weeks (e.g., about 2 weeks, about 4 weeks, about 1 month, about two months, about four months, about six months, about eight months, about ten months, about twelve months) in solution at a temperature of at least 20° C. (e.g., 21° C., 22° C., 23° C., 24° C., 25° C., 26° C., at least 27.5° C., at least 30° C., at least 35° C., at least 40° C., at least 50° C.) and less than 100° C., less than 85° C., less than 75° C., or less than 70° C. The glucagon agonist peptides may include additional modifications that alter its pharmaceutical properties, e.g. increased potency, prolonged half-life in circulation, increased shelf-life, reduced precipitation or aggregation, and/or reduced degradation, e.g., reduced occurrence of cleavage or chemical modification after storage.

In yet further exemplary embodiments, any of the foregoing glucagon agonist peptides can be further modified to improve stability by modifying the amino acid at position 15 of SEQ ID NO: 1 to reduce degradation of the peptide over time, especially in acidic or alkaline buffers. In exemplary embodiments, Asp at position 15 is substituted with a Glu, homo-Glu, cysteic acid, or homo-cysteic acid.

Alternatively, any of the glucagon agonist peptides described herein can be further modified to improve stability by modifying the amino acid at position 16 of SEQ ID NO: 1. In exemplary embodiments, Ser at position 16 is substituted with Thr or Aib, or any of the amino acids substitutions described herein with regard to glucagon agonist peptides which enhance potency at the glucagon receptor. Such modifications reduce cleavage of the peptide bond between Asp15-Ser16.

In some embodiments, any of the glucagon agonist peptides described herein can be further modified to reduce degradation at various amino acid positions by modifying any one, two, three, or all four of positions 20, 21, 24, or 27. Exemplary embodiments include substitution of Gln at position 20 with Ser, Thr, Ala or Aib, substitution of Asp at position 21 with Glu, substitution of Gln at position 24 with Ala or Aib, substitution of Met at position 27 with Leu or Nle. Removal or substitution of methionine reduces degradation due to oxidation of the methionine. Removal or substitution of Gln or Asn reduces degradation due to deamidation of Gln or Asn. Removal or substitution of Asp reduces degradation that occurs through dehydration of Asp to form a cyclic succinimide intermediate followed by isomerization to iso-aspartate.

### Enhanced Potency

In accordance with another embodiment, glucagon agonist peptides are provided that have enhanced potency at the glucagon receptor, wherein the peptides comprise an amino acid modification at position 16 of native glucagon (SEQ ID NO: 1). By way of nonlimiting example, such enhanced potency can be provided by substituting the naturally occurring serine at position 16 with glutamic acid or with another negatively charged amino acid having a side chain with a length of 4 atoms, or alternatively with any one of glutamine, homoglutamic acid, or homocysteic acid, or a charged amino acid having a side chain containing at least one heteroatom, (e.g. N, O, S, P) and with a side chain length of about 4 (or 3-5) atoms. Substitution of serine at position 16 with glutamic acid enhances glucagon activity at least 2-fold, 4-fold, 5-fold and up to 10-fold greater at the glucagon receptor. In some embodiments, the glucagon agonist peptide retains selectivity for the glucagon receptor relative to the GLP-1 receptors, e.g., at least 5-fold, 10-fold, or 15-fold selectivity.

### DPP-IV Resistance

In some embodiments, the glucagon peptides disclosed herein are further modified at position 1 or 2 to reduce susceptibility to cleavage by dipeptidyl peptidase IV. More particularly, in some embodiments, position 1 and/or position 2 of the glucagon agonist peptide is substituted with the DPP-IV resistant amino acid(s) described herein. In some embodiments, position 2 of the analog peptide is substituted with an amino isobutyric acid. In some embodiments, position 2 of the analog peptide is substituted with an amino acid selected from the group consisting of D-serine, D-alanine, glycine, N-methyl serine, and ε-amino butyric acid. In another embodiment, position 2 of the glucagon agonist peptide is substituted with an amino acid selected from the group consisting of D-serine, glycine, and aminoisobutyric acid. In some embodiments, the amino acid at position 2 is not D-serine.

Reduction in glucagon activity upon modification of the amino acids at position 1 and/or position 2 of the glucagon peptide can be restored by stabilization of the alpha-helix structure in the C-terminal portion of the glucagon peptide (around amino acids 12-29). The alpha helix structure can be stabilized by, e.g., formation of a covalent or non-covalent intramolecular bridge (e.g., a lactam bridge between side chains of amino acids at positions “i” and “i+4”, wherein i is an integer from 12 to 25), substitution and/or insertion of amino acids around positions 12-29 with an alpha helix-stabilizing amino acid (e.g., an α,α-disubstituted amino acid such as Aib), as further described herein.

### Modifications at Position 3

Glucagon receptor activity can be reduced by an amino acid modification at position 3 (according to the amino acid numbering of wild type glucagon), e.g. substitution of the naturally occurring glutamine at position 3, with an acidic, basic, or a hydrophobic amino acid. For example substitution at position 3 with glutamic acid, ornithine, or norleucine substantially reduces or destroys glucagon receptor activity. Maintained or enhanced activity at the glucagon receptor may be achieved by modifying the Gln at position 3 with a glutamine analog as described herein. For example, glucagon agonists can comprise the amino acid sequence of SEQ ID NO: 863, SEQ ID NO: 869, SEQ ID NO: 870, SEQ ID NO: 871, SEQ ID NO: 872, SEQ ID NO: 873, and SEQ ID NO: 874.

Additional modifications may be made to the glucagon agonist peptide which may further increase solubility and/or stability and/or glucagon activity. The glucagon agonist peptide may alternatively comprise other modifications that do not substantially affect solubility or stability, and that do not substantially decrease glucagon activity. In exemplary embodiments, the glucagon agonist peptide may comprise a total of up to 11, or up to 12, or up to 13, or up to 14 amino acid modifications relative to the native glucagon sequence. For example, conservative or non-conservative substitutions, additions or deletions may be carried out at any of positions 2, 5, 7, 10, 11, 12, 13, 14, 17, 18, 19, 20, 21, 24, 27, 28 or 29.

Exemplary modifications of the glucagon agonist peptide include but are not limited to:

(a) non-conservative substitutions, conservative substitutions, additions or deletions while retaining at least partial glucagon agonist activity, for example, conservative substitutions at one or more of positions 2, 5, 7, 10, 11, 12, 13, 14, 16, 17, 18, 19, 20, 21, 24, 27, 28 or 29, substitution of Tyr at position 10 with Val or Phe, substitution of Lys at position 12 with Arg, substitution of one or more of these positions with Ala;

(b) deletion of amino acids at positions 29 and/or 28, and optionally position 27, while retaining at least partial glucagon agonist activity;

(c) modification of the aspartic acid at position 15, for example, by substitution with glutamic acid, homoglutamic acid, cysteic acid or homocysteic acid, which may reduce degradation; or modification of the serine at position 16, for example, by substitution of threonine, Aib, glutamic acid or with another negatively charged amino acid having a side chain with a length of 4 atoms, or alternatively with any one of glutamine, homoglutamic acid, or homocysteic acid, which likewise may reduce degradation due to cleavage of the Asp15-Ser16 bond;

(d) addition of a hydrophilic moiety such as the water soluble polymer polyethylene glycol, as described herein, e.g. at position 16, 17, 20, 21, 24, 29, 40 or at the C-terminal amino acid, which may increase solubility and/or half-life;

(e) modification of the methionine at position 27, for example, by substitution with leucine or norleucine, to reduce oxidative degradation;

(f) modification of the Gln at position 20 or 24, e.g. by substitution with Ser, Thr, Ala or Aib, to reduce degradation that occurs through deamidation of Gln

(g) modification of Asp at position 21, e.g. by substitution with Glu, to reduce degradation that occurs through dehydration of Asp to form a cyclic succinimide intermediate followed by isomerization to iso-aspartate;

(h) modifications at position 1 or 2 as described herein that improve resistance to DPP-IV cleavage, optionally in combination with an intramolecular bridge such as a lactam bridge between positions “i” and “i+4”, wherein i is an integer from 12 to 25, e.g., 12, 16, 20, 24;

(i) acylating or alkylating the glucagon peptide as described herein, which may increase the activity at the glucagon receptor and/or the GLP-1 receptor, increase half-life in circulation and/or extending the duration of action and/or delaying the onset of action, optionally combined with addition of a hydrophilic moiety, additionally or alternatively, optionally combined with a modification which selectively reduces activity at the GLP-1 peptide, e.g., a modification of the Thr at position 7, such as a substitution of the Thr at position 7 with an amino acid lacking a hydroxyl group, e.g., Abu or Ile; deleting amino acids C-terminal to the amino acid at position 27 (e.g., deleting one or both of the amino acids at positions 28 and 29, yielding a peptide 27 or 28 amino acids in length);

(j) C-terminal extensions as described herein;

(k) homodimerization or heterodimerization as described herein; and

combinations of the (a) through (k).

In some embodiments, exemplary modifications of the glucagon agonist peptide include at least one amino acid modification selected from Group A and one or more amino acid modifications selected from Group B and/or Group C,

wherein Group A is:

substitution of Asn at position 28 with a charged amino acid;

substitution of Asn at position 28 with a charged amino acid selected from the group consisting of Lys, Arg, His, Asp, Glu, cysteic acid, and homocysteic acid;

substitution at position 28 with Asn, Asp, or Glu;

substitution at position 28 with Asp;

substitution at position 28 with Glu;

substitution of Thr at position 29 with a charged amino acid;

substitution of Thr at position 29 with a charged amino acid selected from the group consisting of Lys, Arg, His, Asp, Glu, cysteic acid, and homocysteic acid;

substitution at position 29 with Asp, Glu, or Lys;

substitution at position 29 with Glu;

insertion of 1-3 charged amino acids after position 29;

insertion after position 29 of Glu or Lys;

insertion after position 29 of Gly-Lys or Lys-Lys;

or combinations thereof;

wherein Group B is:

substitution of Asp at position 15 with Glu;

substitution of Ser at position 16 with Thr or Aib;

and wherein Group C is:

substitution of His at position 1 with a non-native amino acid that reduces susceptibility of the glucagon peptide to cleavage by dipeptidyl peptidase IV (DPP-IV),

substitution of Ser at position 2 with a non-native amino acid that reduces susceptibility of the glucagon peptide to cleavage by dipeptidyl peptidase IV (DPP-IV),

substitution of Lys at position 12 with Arg;

substitution of Gln at position 20 with Ser, Thr, Ala or Aib;

substitution of Asp at position 21 with Glu;

substitution of Gln at position 24 with Ser, Thr, Ala or Aib;

substitution of Met at position 27 with Leu or Nle;

deletion of amino acids at positions 27-29;

deletion of amino acids at positions 28-29;

deletion of the amino acid at positions 29;

or combinations thereof.

In exemplary embodiments, Lys at position 12 is substituted with Arg. In other exemplary embodiments amino acids at positions 29 and/or 28, and optionally at position 27, are deleted.

In some specific embodiments, the glucagon peptide comprises (a) an amino acid modification at position 1 and/or 2 that confers DPP-IV resistance, e.g., substitution with DMIA at position 1, or Aib at position 2, (b) an intramolecular bridge within positions 12-29, e.g. at positions 16 and 20, or one or more substitutions of the amino acids at positions 16, 20, 21, and 24 with an α,α disubstituted amino acid, optionally (c) linked to a hydrophilic moiety such as PEG, e.g., through Cys at position 24, 29 or at the C-terminal amino acid, optionally (d) an amino acid modification at position 27 that substitutes Met with, e.g., Nle, optionally (e) amino acid modifications at positions 20, 21 and 24 that reduce degradation, and optionally (f) linked to SEQ ID NO: 820. When the glucagon peptide is linked to SEQ ID NO: 820, the amino acid at position 29 in certain embodiments is Thr or Gly. In other specific embodiments, the glucagon peptide comprises (a) Asp28Glu29, or Glu28Glu29, or Glu29Glu30, or Glu28Glu30 or Asp28Glu30, and optionally (b) an amino acid modification at position 16 that substitutes Ser with, e.g. Thr or Aib, and optionally (c) an amino acid modification at position 27 that substitutes Met with, e.g., Nle, and optionally (d) amino acid modifications at positions 20, 21 and 24 that reduce degradation. In a specific embodiment, the glucagon peptide is T16, A20, E21, A24, Nle27, D28, E29.

In some embodiments, the glucagon agonist peptide comprises the amino acid sequence:

X1-X2-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Z (SEQ ID NO: 839) with 1 to 3 amino acid modifications thereto,

wherein X1 and/or X2 is a non-native amino acid that reduces susceptibility of (or increases resistance of) the glucagon peptide to cleavage by dipeptidyl peptidase IV (DPP-IV), optionally wherein X1 is selected from the group consisting of His, D-His, N-methyl-His, alpha-methyl-His, imidazole acetic acid, des-amino-His, hydroxyl-His, acetyl-His, homo-His, and alpha, alpha-dimethyl imidiazole acetic acid (DMIA), and X2 is selected from the group consisting of: Ser, D-Ser, D-Ala, Gly, N-methyl-Ser, Val, and alpha, amino isobutyric acid (Aib), wherein at least one of X1 and X2 is a non-native amino acid at that position relative to SEQ ID NO: 1.

wherein Z is selected from the group consisting of —COOH (the naturally occurring C-terminal carboxylate), -Asn-COOH, Asn-Thr-COOH, and Y—COOH, wherein Y is 1 to 2 amino acids, and

optionally wherein an intramolecular bridge, preferably a covalent bond, connects the side chains of an amino acid at position i and an amino acid at position i+4, wherein i is 12, 16, 20 or 24.

In some embodiments, the intramolecular bridge is a lactam bridge. In some embodiments, the amino acids at positions i and i+4 of SEQ ID NO: 839 are Lys and Glu, e.g., Glu16 and Lys20. In some embodiments, X1 is selected from the group consisting of: D-His, N-methyl-His, alpha-methyl-His, imidazole acetic acid, des-amino-His, hydroxyl-His, acetyl-His, homo-His, and alpha, alpha-dimethyl imidiazole acetic acid (DMIA). In other embodiments, X2 is selected from the group consisting of: D-Ser, D-Ala, Gly, N-methyl-Ser, Val, and alpha, amino isobutyric acid (Aib).

In some embodiments, the glucagon peptide is covalently linked to a hydrophilic moiety at any of amino acid positions 16, 17, 20, 21, 24, 29, 40, within a C-terminal extension, or at the C-terminal amino acid. In exemplary embodiments, this hydrophilic moiety is covalently linked to a Lys, Cys, Orn, homocysteine, or acetyl-phenylalanine residue at any of these positions. Exemplary hydrophilic moieties include polyethylene glycol (PEG), for example, of a molecular weight of about 1,000 Daltons to about 40,000 Daltons, or about 20,000 Daltons to about 40,000 Daltons.

In other embodiments, the glucagon agonist peptide comprises the amino acid sequence: X1-X2-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Z (SEQ ID NO: 839),

wherein X1 and/or X2 is a non-native amino acid that reduces susceptibility of (or increases resistance of) the glucagon peptide to cleavage by dipeptidyl peptidase IV (DPP-IV), optionally wherein X1 is selected from the group consisting of His, D-His, N-methyl-His, alpha-methyl-His, imidazole acetic acid, des-amino-His, hydroxyl-His, acetyl-His, homo-His, and alpha, alpha-dimethyl imidiazole acetic acid (DMIA), and X2 is selected from the group consisting of: Ser, D-Ser, D-Ala, Gly, N-methyl-Ser, Val, and alpha, amino isobutyric acid (Aib), wherein at least one of X1 and X2 is a non-native amino acid at that position relative to SEQ ID NO: 1.

wherein one, two, three, four or more of positions 16, 20, 21, and 24 of the glucagon peptide is substituted with an α,α-disubstituted amino acid, and

wherein Z is selected from the group consisting of —COOH (the naturally occurring C-terminal carboxylate), -Asn-COOH, Asn-Thr-COOH, and Y—COOH, wherein Y is 1 to 2 amino acids.

Exemplary further amino acid modifications to the foregoing glucagon agonist peptides include substitution of Thr at position 7 with an amino acid lacking a hydroxyl group, e.g., aminobutyric acid (Abu), Ile, optionally, in combination with substitution or addition of an amino acid comprising a side chain covalently attached (optionally, through a spacer) to an acyl or alkyl group, which acyl or alkyl group is non-native to a naturally-occurring amino acid, substitution of Lys at position 12 with Arg; substitution of Asp at position 15 with Glu; substitution of Ser at position 16 with Thr or Aib; substitution of Gln at position 20 with Ser, Thr, Ala or Aib; substitution of Asp at position 21 with Glu; substitution of Gln at position 24 with Ser, Thr, Ala or Aib; substitution of Met at position 27 with Leu or Nle; substitution of Asn at position 28 with a charged amino acid; substitution of Asn at position 28 with a charged amino acid selected from the group consisting of Lys, Arg, His, Asp, Glu, cysteic acid, and homocysteic acid; substitution at position 28 with Asn, Asp, or Glu; substitution at position 28 with Asp; substitution at position 28 with Glu; substitution of Thr at position 29 with a charged amino acid; substitution of Thr at position 29 with a charged amino acid selected from the group consisting of Lys, Arg, His, Asp, Glu, cysteic acid, and homocysteic acid; substitution at position 29 with Asp, Glu, or Lys; substitution at position 29 with Glu; insertion of 1-3 charged amino acids after position 29; insertion at position 30 (i.e., after position 29) of Glu or Lys; optionally with insertion at position 31 of Lys; addition of SEQ ID NO: 820 to the C-terminus, optionally, wherein the amino acid at position 29 is Thr or Gly; substitution or addition of an amino acid covalently attached to a hydrophilic moiety; or a combination thereof.

Any of the modifications described above in reference to glucagon agonists which increase glucagon receptor activity, retain partial glucagon receptor activity, improve solubility, increase stability, or reduce degradation can be applied to glucagon peptides individually or in combination. Thus, glucagon agonist peptides can be prepared that retain at least 20% of the activity of native glucagon at the glucagon receptor, and which are soluble at a concentration of at least 1 mg/mL at a pH between 6 and 8 or between 6 and 9, (e.g. pH 7), and optionally retain at least 95% of the original peptide (e.g. 5% or less of the original peptide is degraded or cleaved) after 24 hours at 25° C. Alternatively, high potency glucagon peptides can be prepared that exhibit at least about 100%, 125%, 150%, 175%, 200%, 250%, 300%, 350%, 400%, 450%, 500%, 600%, 700%, 800%, 900% or 10-fold or more of the activity of native glucagon at the glucagon receptor, and optionally are soluble at a concentration of at least 1 mg/mL at a pH between 6 and 8 or between 6 and 9, (e.g. pH 7), and optionally retains at least 95% of the original peptide (e.g. 5% or less of the original peptide is degraded or cleaved) after 24 hours at 25° C. In some embodiments, the glucagon peptides described herein may exhibit at least any of the above indicated relative levels of activity at the glucagon receptor but no more than 1,000%, 5,000% or 10,000% of the activity of native glucagon at the glucagon receptor.

### Examples of Embodiments of Glucagon Agonist Peptides

In accordance with some embodiments the native glucagon peptide of SEQ ID NO: 1 is modified by the substitution of the native amino acid at position 28 and/or 29 with a negatively charged amino acid (e.g., aspartic acid or glutamic acid) and optionally the addition of a negatively charged amino acid (e.g., aspartic acid or glutamic acid) to the carboxy terminus of the peptide. In an alternative embodiment the native glucagon peptide of SEQ ID NO: 1 is modified by the substitution of the native amino acid at position 29 with a positively charged amino acid (e.g., lysine, arginine or histidine) and optionally the addition of one or two positively charged amino acid (e.g., lysine, arginine or histidine) on the carboxy terminus of the peptide. In accordance with some embodiments a glucagon analog having improved solubility and stability is provided wherein the analog comprises the amino acid sequence of SEQ ID NO: 834 with the proviso that at least one amino acids at position, 28, or 29 is substituted with an acidic amino acid and/or an additional acidic amino acid is added at the carboxy terminus of SEQ ID NO: 834. In some embodiments the acidic amino acids are independently selected from the group consisting of Asp, Glu, cysteic acid and homocysteic acid.

In accordance with some embodiments a glucagon agonist having improved solubility and stability is provided wherein the agonist comprises the amino acid sequence of SEQ ID NO: 833, wherein at least one of the amino acids at positions 27, 28 or 29 is substituted with a non-native amino acid residue (i.e. at least one amino acid present at position 27, 28 or 29 of the analog is an acid amino acid different from the amino acid present at the corresponding position in SEQ ID NO: 1). In accordance with some embodiments a glucagon agonist is provided comprising the sequence of SEQ ID NO: 833 with the proviso that when the amino acid at position 28 is asparagine and the amino acid at position 29 is threonine, the peptide further comprises one to two amino acids, independently selected from the group consisting of Lys, Arg, His, Asp or Glu, added to the carboxy terminus of the glucagon peptide.

It has been reported that certain positions of the native glucagon peptide can be modified while retaining at least some of the activity of the parent peptide. Accordingly, applicants anticipate that one or more of the amino acids located at positions at positions 2, 5, 7, 10, 11, 12, 13, 14, 16, 17, 18, 19, 20, 21, 24, 27, 28 or 29 of the peptide of SEQ ID NO: 811 can be substituted with an amino acid different from that present in the native glucagon peptide, and still retain the enhanced potency, physiological pH stability and biological activity of the parent glucagon peptide. For example, in accordance with some embodiments the methionine residue present at position 27 of the native peptide is changed to leucine or norleucine to prevent oxidative degradation of the peptide.

In some embodiments a glucagon analog of SEQ ID NO: 833 is provided wherein 1 to 6 amino acids, selected from positions 1, 2, 5, 7, 10, 11, 12, 13, 14, 16, 17, 18, 19, 20, 21 or 24 of the analog differ from the corresponding amino acid of SEQ ID NO: 1. In accordance with another embodiment a glucagon analog of SEQ ID NO: 833 is provided wherein 1 to 3 amino acids selected from positions 1, 2, 5, 7, 10, 11, 12, 13, 14, 16, 17, 18, 19, 20, 21 or 24 of the analog differ from the corresponding amino acid of SEQ ID NO: 1. In another embodiment, a glucagon analog of SEQ ID NO: 807, SEQ ID NO: 808 or SEQ ID NO: 834 is provided wherein 1 to 2 amino acids selected from positions 1, 2, 5, 7, 10, 11, 12, 13, 14, 16, 17, 18, 19, 20, 21 or 24 of the analog differ from the corresponding amino acid of SEQ ID NO: 1, and in a further embodiment those one to two differing amino acids represent conservative amino acid substitutions relative to the amino acid present in the native sequence (SEQ ID NO: 1). In some embodiments a glucagon peptide of SEQ ID NO: 811 or SEQ ID NO: 813 is provided wherein the glucagon peptide further comprises one, two or three amino acid substitutions at positions selected from positions 2, 5, 7, 10, 11, 12, 13, 14, 16, 17, 18, 19, 20, 21, 24, 27 or 29. In some embodiments the substitutions at positions 2, 5, 7, 10, 11, 12, 13, 14, 16, 17, 18, 19, 20, 27 or 29 are conservative amino acid substitutions.

In some embodiments a glucagon agonist is provided comprising an analog peptide of SEQ ID NO: 1 wherein the analog differs from SEQ ID NO: 1 by having an amino acid other than serine at position 2 and by having an acidic amino acid substituted for the native amino acid at position 28 or 29 or an acidic amino acid added to the carboxy terminus of the peptide of SEQ ID NO: 1. In some embodiments the acidic amino acid is aspartic acid or glutamic acid. In some embodiments a glucagon analog of SEQ ID NO: 809, SEQ ID NO: 812, SEQ ID NO: 813 or SEQ ID NO: 832 is provided wherein the analog differs from the parent molecule by a substitution at position 2. More particularly, position 2 of the analog peptide is substituted with an amino acid selected from the group consisting of D-serine, alanine, D-alanine, glycine, n-methyl serine and amino isobutyric acid.

In another embodiment a glucagon agonist is provided comprising an analog peptide of SEQ ID NO: 1 wherein the analog differs from SEQ ID NO: 1 by having an amino acid other than histidine at position 1 and by having an acidic amino acid substituted for the native amino acid at position 28 or 29 or an acidic amino acid added to the carboxy terminus of the peptide of SEQ ID NO: 1. In some embodiments the acidic amino acid is aspartic acid or glutamic acid. In some embodiments a glucagon analog of SEQ ID NO: 809, SEQ ID NO: 812, SEQ ID NO: 813 or SEQ ID NO: 832 is provided wherein the analog differs from the parent molecule by a substitution at position 1. More particularly, position 1 of the analog peptide is substituted with an amino acid selected from the group consisting of DMIA, D-histidine, desaminohistidine, hydroxyl-histidine, acetyl-histidine and homo-histidine.

In accordance with some embodiments the modified glucagon peptide comprises a sequence selected from the group consisting of SEQ ID NO: 809, SEQ ID NO: 812, SEQ ID NO: 813 and SEQ ID NO: 832. In a further embodiment a glucagon peptide is provided comprising a sequence of SEQ ID NO: 809, SEQ ID NO: 812, SEQ ID NO: 813 or SEQ ID NO: 832 further comprising one to two amino acids, added to the C-terminus of SEQ ID NO: 809, SEQ ID NO: 812, SEQ ID NO: 813 or SEQ ID NO: 832, wherein the additional amino acids are independently selected from the group consisting of Lys, Arg, His, Asp Glu, cysteic acid or homocysteic acid. In some embodiments the additional amino acids added to the carboxy terminus are selected from the group consisting of Lys, Arg, His, Asp or Glu or in a further embodiment the additional amino acids are Asp or Glu.

In another embodiment the glucagon peptide comprises the sequence of SEQ ID NO: 807 or a glucagon agonist analog thereof. In some embodiments the peptide comprising a sequence selected from the group consisting of SEQ ID NO: 808, SEQ ID NO: 810, SEQ ID NO: 811, SEQ ID NO: 812 and SEQ ID NO: 813. In another embodiment the peptide comprising a sequence selected from the group consisting of SEQ ID NO: 808, SEQ ID NO: 810 and SEQ ID NO: 811. In some embodiments the glucagon peptide comprises the sequence of SEQ ID NO: 808, SEQ ID NO: 810 and SEQ ID NO: 811 further comprising an additional amino acid, selected from the group consisting of Asp and Glu, added to the C-terminus of the glucagon peptide. In some embodiments the glucagon peptide comprises the sequence of SEQ ID NO: 811 or SEQ ID NO: 813, and in a further embodiment the glucagon peptide comprises the sequence of SEQ ID NO: 811.

In accordance with some embodiments a glucagon agonist is provided comprising a modified glucagon peptide selected from the group consisting of:

(SEQ ID NO: 834)
NH₂-His-Ser-Gln-Gly-Thr-Phe-Thr-Ser-Asp-Tyr-Ser-
Lys-Tyr-Leu-Xaa-Ser-Arg-Arg-Ala-Gln-Asp-Phe-Val-
Gln-Trp-Leu-Xaa-Xaa-Xaa-R,
(SEQ ID NO: 811)
NH₂-His-Ser-Gln-Gly-Thr-Phe-Thr-Ser-Asp-Tyr-Ser-
Lys-Tyr-Leu-Asp-Ser-Arg-Arg-Ala-Gln-Asp-Phe-Val-
Gln-Trp-Leu-Met-Asp-Thr-R
and
(SEQ ID NO: 813)
NH₂-His-Ser-Gln-Gly-Thr-Phe-Thr-Ser-Asp-Tyr-Ser-
Lys-Tyr-Leu-Glu-Ser-Arg-Arg-Ala-Gln-Asp-Phe-Val-
Gln-Trp-Leu-Met-Asp-Thr-R

Wherein the Xaa at position 15 is Asp, Glu, cysteic acid, homoglutamic acid or homocysteic acid, the Xaa at position 27 is Met, Leu or Nle, the Xaa at position 28 is Asn or an acidic amino acid and the Xaa at position 29 is Thr or an acidic amino acid and R is an acidic amino acid, COOH or CONH₂, with the proviso that an acidic acid residue is present at one of positions 28, 29 or 30. In some embodiments R is COOH, and in another embodiment R is CONH₂.

The present disclosure also encompasses glucagon fusion peptides wherein a second peptide has been fused to the C-terminus of the glucagon peptide to enhance the stability and solubility of the glucagon peptide. More particularly, the fusion glucagon peptide may comprise a glucagon agonist analog comprising a glucagon peptide NH₂-His-Ser-Gln-Gly-Thr-Phe-Thr-Ser-Asp-Tyr-Ser-Lys-Tyr-Leu-Xaa-Ser-Arg-Arg-Ala-Gln-Asp-Phe-Val-Gln-Trp-Leu-Xaa-Xaa-Xaa-R (SEQ ID NO: 834), wherein R is an acidic amino acid or an amino acid sequence of SEQ ID NO: 820 (GPSSGAPPPS), SEQ ID NO: 821 (KRNRNNIA) or SEQ ID NO: 822 (KRNR) linked to the carboxy terminal amino acid of the glucagon peptide. In some embodiments the glucagon peptide is selected from the group consisting of SEQ ID NO: 833, SEQ ID NO: 807 or SEQ ID NO: 808 further comprising an amino acid sequence of SEQ ID NO: 820 (GPSSGAPPPS), SEQ ID NO: 821 (KRNRNNIA) or SEQ ID NO: 822 (KRNR) linked to the carboxy terminal amino acid of the glucagon peptide. In some embodiments the glucagon fusion peptide comprises SEQ ID NO: 802, SEQ ID NO: 803, SEQ ID NO: 804, SEQ ID NO: 805 and SEQ ID NO: 806 or a glucagon agonist analog thereof, further comprising an amino acid sequence of SEQ ID NO: 820 (GPSSGAPPPS), SEQ ID NO: 821 (KRNRNNIA) or SEQ ID NO: 822 (KRNR) linked to amino acid 29 of the glucagon peptide. In accordance with some embodiments the fusion peptide further comprises a PEG chain linked to an amino acid at position 16, 17, 21, 24, 29, within a C-terminal extension, or at the C-terminal amino acid, wherein the PEG chain is selected from the range of 500 to 40,000 Daltons. In some embodiments the amino acid sequence of SEQ ID NO: 820 (GPSSGAPPPS), SEQ ID NO: 821 (KRNRNNIA) or SEQ ID NO: 822 (KRNR) is bound to amino acid 29 of the glucagon peptide through a peptide bond. In some embodiments the glucagon peptide portion of the glucagon fusion peptide comprises a sequence selected from the group consisting of SEQ ID NO: 810, SEQ ID NO: 811 and SEQ ID NO: 813. In some embodiments the glucagon peptide portion of the glucagon fusion peptide comprises the sequence of SEQ ID NO: 811 or SEQ ID NO: 813, wherein a PEG chain is linked at position 21, 24, 29, within a C-terminal extension or at the C-terminal amino acid, respectively.

In another embodiment the glucagon peptide sequence of the fusion peptide comprises the sequence of SEQ ID NO: 811, further comprising an amino acid sequence of SEQ ID NO: 820 (GPSSGAPPPS), SEQ ID NO: 821 (KRNRNNIA) or SEQ ID NO: 822 (KRNR) linked to amino acid 29 of the glucagon peptide. In some embodiments the glucagon fusion peptide comprises a sequence selected from the group consisting of SEQ ID NO: 824, SEQ ID NO: 825 and SEQ ID NO: 826. Typically the fusion peptides of the present invention will have a C-terminal amino acid with the standard carboxylic acid group. However, analogs of those sequences wherein the C-terminal amino acid has an amide substituted for the carboxylic acid are also encompassed as embodiments. In accordance with some embodiments the fusion glucagon peptide comprises a glucagon agonist analog selected from the group consisting of SEQ ID NO: 810, SEQ ID NO: 811 and SEQ ID NO: 813, further comprising an amino acid sequence of SEQ ID NO: 823 (GPSSGAPPPS-CONH₂) linked to amino acid 29 of the glucagon peptide.

The glucagon agonists of the present invention can be further modified to improve the peptide's solubility and stability in aqueous solutions while retaining the biological activity of the glucagon peptide. In accordance with some embodiments, introduction of hydrophilic groups at one or more positions selected from positions 16, 17, 20, 21, 24 and 29 of the peptide of SEQ ID NO: 811, or a glucagon agonist analog thereof, are anticipated to improve the solubility and stability of the pH stabilize glucagon analog. More particularly, in some embodiments the glucagon peptide of SEQ ID NO: 810, SEQ ID NO: 811, SEQ ID NO: 813, or SEQ ID NO: 832 is modified to comprise one or more hydrophilic groups covalently linked to the side of amino acids present at positions 21 and 24 of the glucagon peptide.

In accordance with some embodiments, the glucagon peptide of SEQ ID NO: 811 is modified to contain one or more amino acid substitution at positions 16, 17, 20, 21, 24 and/or 29, wherein the native amino acid is substituted with an amino acid having a side chain suitable for crosslinking with hydrophilic moieties, including for example, PEG. The native peptide can be substituted with a naturally occurring amino acid or a synthetic (non-naturally occurring) amino acid. Synthetic or non-naturally occurring amino acids refer to amino acids that do not naturally occur in vivo but which, nevertheless, can be incorporated into the peptide structures described herein.

In some embodiments, a glucagon agonist of SEQ ID NO: 810, SEQ ID NO: 811 or SEQ ID NO: 813 is provided wherein the native glucagon peptide sequence has been modified to contain a naturally occurring or synthetic amino acid in at least one of positions 16, 17, 21, 24, 29, within a C-terminal extension or at the C-terminal amino acid of the native sequence, wherein the amino acid substitute further comprises a hydrophilic moiety. In some embodiments the substitution is at position 21 or 24, and in a further embodiment the hydrophilic moiety is a PEG chain. In some embodiments the glucagon peptide of SEQ ID NO: 811 is substituted with at least one cysteine residue, wherein the side chain of the cysteine residue is further modified with a thiol reactive reagent, including for example, maleimido, vinyl sulfone, 2-pyridylthio, haloalkyl, and haloacyl. These thiol reactive reagents may contain carboxy, keto, hydroxyl, and ether groups as well as other hydrophilic moieties such as polyethylene glycol units. In an alternative embodiment, the native glucagon peptide is substituted with lysine, and the side chain of the substituting lysine residue is further modified using amine reactive reagents such as active esters (succinimido, anhydride, etc) of carboxylic acids or aldehydes of hydrophilic moieties such as polyethylene glycol. In some embodiments the glucagon peptide is selected form the group consisting of SEQ ID NO: 814, SEQ ID NO: 815, SEQ ID NO: 816, SEQ ID NO: 817, SEQ ID NO: 818 and SEQ ID NO: 819.

In accordance with some embodiments the pegylated glucagon peptide comprises two or more polyethylene glycol chains covalently bound to the glucagon peptide wherein the total molecular weight of the glucagon chains is about 1,000 to about 5,000 Daltons. In some embodiments the pegylated glucagon agonist comprises a peptide of SEQ ID NO: 806, wherein a PEG chain is covalently linked to the amino acid residue at position 21 and at position 24, and wherein the combined molecular weight of the two PEG chains is about 1,000 to about 5,000 Daltons. In another embodiment the pegylated glucagon agonist comprises a peptide of SEQ ID NO: 806, wherein a PEG chain is covalently linked to the amino acid residue at position 21 and at position 24, and wherein the combined molecular weight of the two PEG chains is about 5,000 to about 20,000 Daltons.

The polyethylene glycol chain may be in the form of a straight chain or it may be branched. In accordance with some embodiments the polyethylene glycol chain has an average molecular weight selected from the range of about 500 to about 40,000 Daltons. In some embodiments the polyethylene glycol chain has a molecular weight selected from the range of about 500 to about 5,000 Daltons. In another embodiment the polyethylene glycol chain has a molecular weight of about 20,000 to about 40,000 Daltons.

Any of the glucagon peptides described above may be further modified to include a covalent or non-covalent intramolecular bridge or an alpha helix-stabilizing amino acid within the C-terminal portion of the glucagon peptide (amino acid positions 12-29). In accordance with some embodiments, the glucagon peptide comprises any one or more of the modifications discussed above in addition to an amino acid substitution at positions 16, 20, 21, or 24 (or a combination thereof) with an α,α-disubstituted amino acid, e.g., Aib. In accordance with another embodiment, the glucagon peptide comprises any one or more modifications discussed above in addition to an intramolecular bridge, e.g., a lactam, between the side chains of the amino acids at positions 16 and 20 of the glucagon peptide.

In accordance with some embodiments, the glucagon peptide comprises the amino acid sequence of SEQ ID NO: 877, wherein the Xaa at position 3 is an amino acid comprising a side chain of Structure I, II, or III:

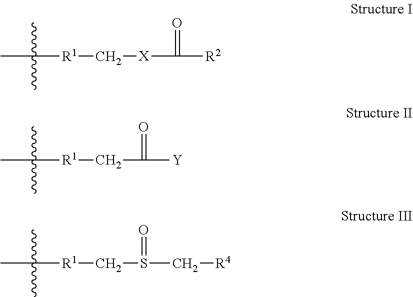

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, O, or S; and Y is NHR⁴, SR³, or OR³. In some embodiments, X is NH or Y is NHR⁴. In some embodiments, R¹ is C₀₋₂ alkyl or C₁ heteroalkyl. In some embodiments, R² is NHR⁴ or C₁ alkyl. In some embodiments, R⁴ is H or C¹ alkyl. In exemplary embodiments an amino acid comprising a side chain of Structure I is provided wherein, R¹ is CH₂—S, X is NH, and R² is CH₃ (acetamidomethyl-cysteine, C(Acm)); R¹ is CH₂, X is NH, and R² is CH₃ (acetyldiaminobutanoic acid, Dab(Ac)); R¹ is C₀ alkyl, X is NH, R² is NHR⁴, and R⁴ is H (carbamoyldiaminopropanoic acid, Dap(urea)); or R¹ is CH₂—CH₂, X is NH, and R² is CH₃ (acetylornithine, Orn(Ac)). In exemplary embodiments an amino acid comprising a side chain of Structure II is provided, wherein R¹ is CH₂, Y is NHR⁴, and R⁴ is CH₃ (methylglutamine, Q(Me)); In exemplary embodiments an amino acid comprising a side chain of Structure III is provided wherein, R¹ is CH₂ and R⁴ is H (methionine-sulfoxide, M(O)); In specific embodiments, the amino acid at position 3 is substituted with Dab(Ac). For example, glucagon agonists can comprise the amino acid sequence of SEQ ID NO: 863, SEQ ID NO: 869, SEQ ID NO: 871, SEQ ID NO: 872, SEQ ID NO: 873, and SEQ ID NO: 874.

In certain embodiments, the glucagon peptide is an analog of the glucagon peptide of SEQ ID NO: 877. In specific aspects, the analog comprises any of the amino acid modifications described herein, including, but not limited to: a substitution of Asn at position 28 with a charged amino acid; a substitution of Asn at position 28 with a charged amino acid selected from the group consisting of Lys, Arg, His, Asp, Glu, cysteic acid, and homocysteic acid; a substitution at position 28 with Asn, Asp, or Glu; a substitution at position 28 with Asp; a substitution at position 28 with Glu; a substitution of Thr at position 29 with a charged amino acid; a substitution of Thr at position 29 with a charged amino acid selected from the group consisting of Lys, Arg, His, Asp, Glu, cysteic acid, and homocysteic acid; a substitution at position 29 with Asp, Glu, or Lys; a substitution at position 29 with Glu; a insertion of 1-3 charged amino acids after position 29; an insertion after position 29 of Glu or Lys; an insertion after position 29 of Gly-Lys or Lys-Lys; and a combination thereof.

In certain embodiments, the analog of the glucagon peptide of SEQ ID NO: 877 comprises an α,α-disubstituted amino acid, such as Aib, at one, two, three, or all of positions 16, 20, 21, and 24.

In certain embodiments, the analog of the glucagon peptide of SEQ ID NO: 877 comprises one or more of the following: substitution of His at position 1 with a non-native amino acid that reduces susceptibility of the glucagon peptide to cleavage by dipeptidyl peptidase IV (DPP-IV), substitution of Ser at position 2 with a non-native amino acid that reduces susceptibility of the glucagon peptide to cleavage by dipeptidyl peptidase IV (DPP-IV), substitution of Thr at position 7 with an amino acid lacking a hydroxyl group, e.g., Abu or Ile; substitution of Tyr at position 10 with Phe or Val; substitution of Lys at position 12 with Arg; substitution of Asp at position 15 with Glu, substitution of Ser at position 16 with Thr or Aib; substitution of Gln at position 20 with Ala or Aib; substitution of Asp at position 21 with Glu; substitution of Gln at position 24 with Ala or Aib; substitution of Met at position 27 with Leu or Nle; deletion of amino acids at positions 27-29; deletion of amino acids at positions 28-29; deletion of the amino acid at positions 29; addition of the amino acid sequence of SEQ ID NO: 820 to the C-terminus, wherein the amino acid at position 29 is Thr or Gly, or a combination thereof.

In accordance with specific embodiments, the glucagon peptide comprises the amino acid sequence of any of SEQ ID NOs: 862-867 and 869-874.

In certain embodiments, the analog of the glucagon peptide comprising SEQ ID NO: 877 comprises a hydrophilic moiety, e.g., PEG, covalently linked to the amino acid at any of positions 16, 17, 20, 21, 24, and 29 or at the C-terminal amino acid.

In certain embodiments, the glucagon agonist peptide comprises the sequence of SEQ ID NO: 877 wherein an amino acid comprising a side chain is covalently attached, optionally through a spacer, to an acyl group or an alkyl group, which acyl group or alkyl group is non-native to a naturally-occurring amino acid. In one embodiment the covalently linked acyl or alkyl group has a carboxylate at its free end. The acyl group in some embodiments is a C4 to C30 fatty acyl group, optionally with carboxylate groups at each end. In one embodiment the glucagon agonist peptide comprises a covalently linked C4 to C30 acyl group optionally with a carboxylate at its free end. In specific aspects, the acyl group or alkyl group is covalently attached to the side chain of the amino acid at position 10. In some embodiments, the amino acid at position 7 is Be or Abu.

The glucagon agonist may be a peptide comprising the amino acid sequence of any of the SEQ ID NOs: 1-919, optionally with up to 1, 2, 3, 4, or 5 further modifications that retain glucagon agonist activity. In certain embodiments, the glucagon agonist comprises the amino acids of any of SEQ ID NOs: 859-919.

In accordance with one embodiment Q is a glucagon analog comprising the sequence X₁X₂X₃GTFTSDYSX₁₂YLX₁₅X₁₆RRAQX₂₁FVX₂₁WLX₂₇X₂₈X₂₉ (SEQ ID NO: 920)

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₃ is an amino acid comprising a side chain of Structure I, II, or III:

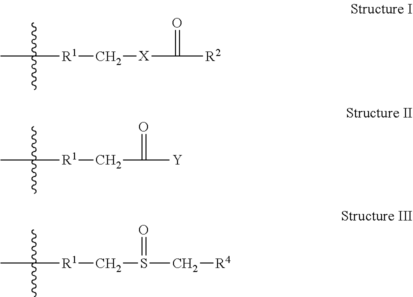

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, O, or S; and Y is NHR⁴, SR³, or OR³;

X₁₂ is Lys or Arg;

X₁₅ is Asp, Glu, cysteic acid, homoglutamic acid or homocysteic acid;

X₁₆ is Ser, glutamine, homoglutamic acid, homocysteic acid, Thr or Aib,

X₂₁ is Asp, Lys, Cys, Orn, homocysteine or acetyl phenylalanine;

X₂₄ is Gln, Lys, Cys, Orn, homocysteine or acetyl phenylalanine;

X₂₇ is Met, Leu or Nle;

X₂₈ is Asn, Lys, Arg, His, Asp or Glu; and

X₂₉ is Thr, Lys, Arg, His, Gly, Asp or Glu, optionally with up to 3 additional conservative amino acid substitutions at positions selected from 5, 7, 10, 11, 13, 14, 17, 18, 19, or 20, and optionally wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) is bound to amino acid 29 of the glucagon peptide through a peptide bond, wherein X₄₀ is an amino acid selected from the group consisting of Cys or Lys.

In accordance with one embodiment Q is a glucagon analog comprising the sequence

HX₂QGTFTSDYSX₁₂YLX₁₅X₁₆RRAQX₂₁FVX₂₄WLX₂₇X₂₈X₂₉ (SEQ ID NO: 921)

wherein

X₂ is selected from the group consisting of Ser, D-Ser, Ala, D-Ala, Gly, N-methyl-Ser, Aib, Val, or α-amino-N-butyric acid;

X₁₂ is Lys or Arg;

X₁₅ is Asp or Glu;

X₁₆ is Ser, Thr or Aib,

X₂₁ is Asp, Lys, Cys, Orn, homocysteine or acetyl phenylalanine;

X₂₄ is Gln, Lys, Cys, Orn, homocysteine or acetyl phenylalanine;

X₂₇ is Met, Leu or Nle;

X₂₈ is Asn, Lys, Arg, His, Asp or Glu; and

X₂₉ is Thr, Lys, Arg, His, Gly, Asp or Glu, optionally wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) is bound to amino acid 29 of the glucagon peptide through a peptide bond, wherein X₄₀ is an amino acid selected from the group consisting of Cys or Lys.

In accordance with one embodiment Q is a glucagon analog comprising the sequence HX₂QGTFTSDYSX₁₂YLX₁₅X₁₆RRAQDFVQWLX₂₇X₂₈X₂₉ (SEQ ID NO: 922)

wherein
X₂ is selected from the group consisting of Ser, D-Ser, Ala, D-Ala, Gly, N-methyl-Ser, Aib, Val, or α-amino-N-butyric acid;

### X₁₂ is Lys or Arg;

### X₁₅ is Asp or Glu;

### X₁₆ is Ser, Thr or Aib,

### X₂₇ is Met, Leu or Nle;

### X₂₈ is Asn, Lys, Arg, His, Asp or Glu; and

X₂₉ is Thr, Lys, Arg, His, Gly, Asp or Glu, optionally wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) is bound to amino acid 29 of the glucagon peptide through a peptide bond, wherein X₄₀ is an amino acid selected from the group consisting of Cys or Lys.

In accordance with one embodiment Q is a glucagon analog comprising the sequence of SEQ ID NO: 1 modified by comprising at least one amino acid modification selected from the group consisting of a substitution at position 28 with Asn, Asp, or Glu;

substitution at position 28 with Asp;

substitution at position 28 with Glu;

substitution of Thr at position 29 with a charged amino acid;

substitution of Thr at position 29 with a charged amino acid selected from the group consisting of Lys, Arg, His, Asp, Glu, cysteic acid, and homocysteic acid;

substitution at position 29 with Asp, Glu, or Lys;

substitution at position 29 with Glu or Gly;

insertion of 1-3 charged amino acids after position 29;

insertion after position 29 of Glu or Lys; or insertion after position 29 of Gly-Lys or Lys-Lys, optionally wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) is bound to amino acid 29 of the glucagon peptide through a peptide bond, wherein X₄₀ is an amino acid selected from the group consisting of Cys or Lys. In a further embodiment Q is a glucagon analog comprising the sequence HX₂QGTFTSDYSX₁₂YLX₁₅X₁₆RRAQDFVQWLX₂₇X₂₈GGPSSGAPPPSX₄₀ (SEQ ID NO: 923) wherein

X₂ is selected from the group consisting of Ser, D-Ser, Ala, D-Ala, Gly, N-methyl-Ser, Aib, Val, or α-amino-N-butyric acid;

X₁₂ is Lys or Arg;

X₁₅ is Asp or Glu;

X₁₆ is Ser, Thr or Aib,

X₂₇ is Met, Leu or Nle;

X₂₈ is Asn, Lys, Arg, His, Asp or Glu; and

X₄₀ is Cys or Lys. In one embodiment the glucagon peptide is SEQ ID NO: 923, wherein X₂ of is Aib or D-Ser and X₁₆ is Aib.

In accordance with one embodiment Q is a glucagon analog comprising the sequence HX₂QGTFTSDYSX₁₂YLDSRRAQDFVQWLX₂₇X₂₈GGPSSGAPPPSX₄₀ (SEQ ID NO: 924)

wherein

X₂ is selected from the group consisting of Ser, D-Ser, Ala, D-Ala, Gly, N-methyl-Ser, Aib, Val, or α-amino-N-butyric acid;

X₁₂ is Lys or Arg;

X₂₇ is Met, Leu or Nle;

X₂₈ is Asn, Lys, Arg, His, Asp or Glu; and

X₄₀ is an amino acid selected from the group consisting of Cys or Lys. In one embodiment X₂ of SEQ ID NO: 924 is Aib or D-Ser.

In one embodiment Q comprises the amino acid sequence:

His-X₂-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Z (SEQ ID NO: 839) with 1 to 3 amino acid modifications thereto,
(a) wherein X₂ is a non-native amino acid (relative to SEQ ID NO: 1) that reduces susceptibility of the glucagon peptide to cleavage by dipeptidyl peptidase IV (DPP-IV),
(b) wherein Z is selected from the group consisting of —COOH, -Asn-COOH, Asn-Thr-COOH, and W—COOH, wherein W is selected from the group consisting of GPSSGAPPPS, GGPSSGAPPPS, NGGPSSGAPPPS and NGGPSSGAPPPSK, wherein Q exhibits glucagon agonist activity. In one embodiment Q comprises the amino acid sequence:
His-X₂-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Gly-Gly-Pro-Ser-Ser-Gly-Ala-Pro-Pro-Pro-X₄₀ (SEQ ID NO: 935) wherein X₂ is Aib or D-Ser and X₄₀ is Lys or Cys. In one embodiment Q comprises the peptide of SEQ ID NO: 935 wherein X₂ is D-Ser and X₄₀ is Lys.

In accordance with one embodiment L-Y is covalently conjugated to the N-terminus, C-terminus, or an amino acid side chain of Q. More particularly, L-Y is covalently conjugated to an amino acid side chain of an amino acid at position 10, 30, 37, 38, 39, 40, 41, 42, or 43 of Q, and L is an amino acid or dipeptide. In one embodiment the carboxylate group of 3,5,3′,5′-tetra-iodothyronine or 3,5,3′-triiodo L-thyronine is covalently linked to an amine of Q to form an amide bond.

### Structure of L

In some embodiments, L is a bond. In these embodiments, Q and Y are conjugated together by reacting a nucleophilic reactive moiety on Q with and electrophilic reactive moiety on Y. In alternative embodiments, Q and Y are conjugated together by reacting an electrophilic reactive moiety on Q with a nucleophilic moiety on Y. In exemplary embodiments, L is an amide bond that forms upon reaction of an amine on Q (e.g. an ε-amine of a lysine residue) with a carboxyl group on Y. In alternative embodiments, Q and or Y are derivatized with a derivatizing agent before conjugation.

In some embodiments, L is a linking group. In some embodiments, L is a bifunctional linker and comprises only two reactive groups before conjugation to Q and Y. In embodiments where both Q and Y have electrophilic reactive groups, L comprises two of the same or two different nucleophilic groups (e.g. amine, hydroxyl, thiol) before conjugation to Q and Y. In embodiments where both Q and Y have nucleophilic reactive groups, L comprises two of the same or two different electrophilic groups (e.g. carboxyl group, activated form of a carboxyl group, compound with a leaving group) before conjugation to Q and Y. In embodiments where one of Q or Y has a nucleophilic reactive group and the other of Q or Y has an electrophilic reactive group, L comprises one nucleophilic reactive group and one electrophilic group before conjugation to Q and Y.

L can be any molecule with at least two reactive groups (before conjugation to Q and Y) capable of reacting with each of Q and Y. In some embodiments L has only two reactive groups and is bifunctional. L (before conjugation to the peptides) can be represented by Formula VI:

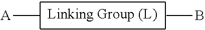

wherein A and B are independently nucleophilic or electrophilic reactive groups. In some embodiments A and B are either both nucleophilic groups or both electrophilic groups. In some embodiments one of A or B is a nucleophilic group and the other of A or B is an electrophilic group.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some embodiments, the linking group is hydrophilic such as, for example, polyalkylene glycol. Before conjugation to the peptides of the composition, the hydrophilic linking group comprises at least two reactive groups (A and B), as described herein and as shown below:

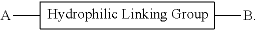

In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on Q or Y and the carboxylic acid or activated carboxylic acid can be coupled to an amine on Q or Y with or without the use of a coupling reagent. Any appropriate coupling agent known to one skilled in the art can be used to couple the carboxylic acid with the amine. In some embodiments, the linking group is maleimido-PEG(20 kDa)-COOH, iodoacetyl-PEG(20 kDa)-COOH, maleimido-PEG(20 kDa)-NHS, or iodoacetyl-PEG(20 kDa)-NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimido, disulfide, amide, ester, thioester, alkene, cycloalkene, alkyne, trizoyl, carbamate, carbonate, cathepsin B-cleavable, and hydrazone. In some embodiments, the linking group is an amino acid selected from the group Asp, Glu, homoglutamic acid, homocysteic acid, cysteic acid, gamma-glutamic acid. In some embodiments, the linking group is a dipeptide selected from the group consisting of: Ala-Ala, β-Ala-β-Ala, Leu-Leu, Pro-Pro, γ-aminobutyric acid-γ-aminobutyric acid, and γ-Glu-γ-Glu. In one embodiment L comprises gamma-glutamic acid.

In embodiments where Q and Y are conjugated together by reacting a carboxylic acid with an amine, an activating agent can be used to form an activated ester of the carboxylic acid. The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3-tetramethyl uronium hexafluorophosphate (HATU), and o-benzotriazole-N,N,N′,N′-tetramethyl-uronium-hexafluoro-phosphate (HBTU).

In some embodiments, Q comprises a nucleophilic reactive group (e.g. the amino group, thiol group, or hydroxyl group of the side chain of lysine, cysteine or serine) that is capable of conjugating to an electrophilic reactive group on Y or L. In some embodiments, Q comprises an electrophilic reactive group (e.g. the carboxylate group of the side chain of Asp or Glu) that is capable of conjugating to a nucleophilic reactive group on Y or L. In some embodiments, Q is chemically modified to comprise a reactive group that is capable of conjugating directly to Y or to L. In some embodiments, Q is modified at the C-terminal to comprise a natural or nonnatural amino acid with a nucleophilic side chain, such as an amino acid represented by Formula I, Formula II, or Formula III, as previously described herein (see Acylation and alkylation). In exemplary embodiments, the C-terminal amino acid of Q is selected from the group consisting of lysine, ornithine, serine, cysteine, and homocysteine. For example, the C-terminal amino acid of Q can be modified to comprise a lysine residue. In some embodiments, Q is modified at the C-terminal amino acid to comprise a natural or nonnatural amino acid with an electrophilic side chain such as, for example, Asp and Glu. In some embodiments, an internal amino acid of Q is substituted with a natural or nonnatural amino acid having a nucleophilic side chain, such as an amino acid represented by Formula I, Formula II, or Formula III, as previously described herein (see Acylation and alkylation). In exemplary embodiments, the internal amino acid of Q that is substituted is selected from the group consisting of lysine, ornithine, serine, cysteine, and homocysteine. For example, an internal amino acid of Q can be substituted with a lysine residue. In some embodiments, an internal amino acid of Q is substituted with a natural or nonnatural amino acid with an electrophilic side chain, such as, for example, Asp and Glu.

In some embodiments, Y comprises a reactive group that is capable of conjugating directly to Q or to L. In some embodiments, Y comprises a nucleophilic reactive group (e.g. amine, thiol, hydroxyl) that is capable of conjugating to an electrophilic reactive group on Q or L. In some embodiments, Y comprises electrophilic reactive group (e.g. carboxyl group, activated form of a carboxyl group, compound with a leaving group) that is capable of conjugating to a nucleophilic reactive group on Q or L.

Stability of L in vivo

In some embodiments, L is stable in vivo. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers. For example, the following compound is not capable of undergoing significant hydrolysis in vivo:

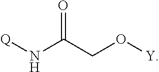

In some embodiments, L is hydrolyzable in vivo. In these embodiments, L comprises a functional group that is capable of undergoing hydrolysis in vivo. Nonlimiting examples of functional groups that are capable of undergoing hydrolysis in vivo include esters, anhydrides, and thioesters. For example the following compound is capable of undergoing hydrolysis in vivo because it comprises an ester group:

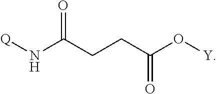

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the Q-L-Y conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

In accordance with one embodiment L-Y comprises the structure:

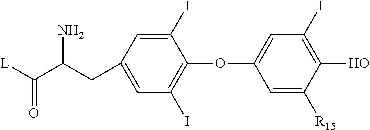

wherein

L is a bond, an amino acid, or dipeptide joining Q to Y; and

R₁₅ is H or I. In one embodiment L is γ-Glu or the dipeptide, γ-Glu-γ-Glu. In one embodiment L-Y comprises the structure

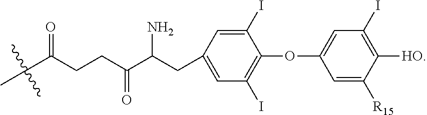

### Acylation of Q

In some embodiments, the glucagon agonist peptide, Q is modified to comprise an acyl group. The acyl group can be covalently linked directly to an amino acid of the peptide Q, or indirectly to an amino acid of Q via a spacer, wherein the spacer is positioned between the amino acid of Q and the acyl group. Q may be acylated at the same amino acid position where a hydrophilic moiety is linked, or at a different amino acid position. The glucagon agonist peptide may comprise an acyl group which is non-native to a naturally-occurring amino acid. Acylation can be carried out at any position within Q. Acylation may occur at any position including any of positions 1-29, a position within a C-terminal extension, or the C-terminal amino acid, provided that the activity exhibited by the non-acylated glucagon agonist peptide is retained upon acylation. For example, if the unacylated peptide has glucagon agonist activity, then the acylated peptide retains the glucagon agonist activity. Nonlimiting examples include acylation at positions 5, 7, 10, 11, 12, 13, 14, 16, 17, 18, 19, 20, 21, 24, 27, 28, or 29 (according to the amino acid numbering of wild type glucagon) or at positions 30, 37, 38, 39, 40, 41, 42, or 43 of a C-terminal extended glucagon agonist peptide (according to the amino acid numbering of wild type glucagon). Other nonlimiting examples include acylation at position 10 (according to the amino acid numbering of the wild type glucagon) and pegylation at one or more positions in the C-terminal portion of the glucagon peptide, e.g., position 24, 28 or 29 (according to the amino acid numbering of the wild type glucagon), within a C-terminal extension, or at the C-terminus (e.g., through adding a C-terminal Cys). In one embodiment the acyl group is a C4 to C30 fatty acyl group, optionally with carboxylate groups at each end. In other embodiments, the acyl group is a C16, C18 or C20 acyl group optionally with a carboxylate at its free end when linked to the glucagon agonist peptide.

In a specific aspect of the invention, Q is modified to comprise an acyl group by direct acylation of an amine, hydroxyl, or thiol of a side chain of an amino acid of Q. In some embodiments, Q is directly acylated through the side chain amine, hydroxyl, or thiol of an amino acid. In some embodiments, acylation is at position 10, 20, 24, or 29 (according to the amino acid numbering of the wild type glucagon). In this regard, the acylated glucagon agonist peptide can comprise the amino acid sequence of SEQ ID NO: 1, or a modified amino acid sequence thereof comprising one or more of the amino acid modifications described herein, with at least one of the amino acids at positions 10, 20, 24, and 29 (according to the amino acid numbering of the wild type glucagon) modified to any amino acid comprising a side chain amine, hydroxyl, or thiol. In some specific embodiments of the invention the direct acylation of the Q occurs through the side chain amine, hydroxyl, or thiol of the amino acid at position 10 (according to the amino acid numbering of the wild type glucagon).

In some embodiments, the acylated amino acid of Q comprises a side chain amine and is an amino acid of Formula I:

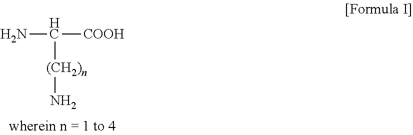

In some exemplary embodiments, the amino acid of Formula I, is the amino acid wherein n is 4 (Lys) or n is 3 (Orn).

In other embodiments, the acylated amino acid of Q comprises a side chain hydroxyl and is an amino acid of Formula II:

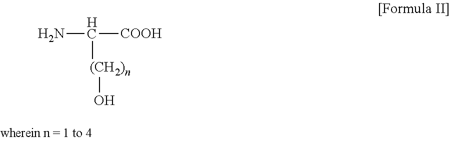

In some exemplary embodiments, the amino acid of Formula II is the amino acid wherein n is 1 (Ser).

In yet other embodiments, the acylated amino acid of peptide Q comprises a side chain thiol and is an amino acid of Formula III:

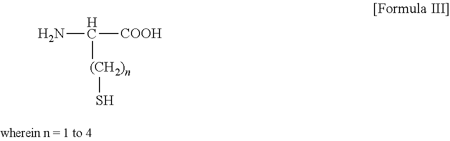

In some exemplary embodiments, the amino acid of Formula III is the amino acid wherein n is 1 (Cys).

In yet other embodiments, the amino acid of peptide Q comprising a side chain amine, hydroxyl, or thiol is a disubstituted amino acid comprising the same structure of Formula I, Formula II, or Formula III, except that the hydrogen bonded to the alpha carbon of the amino acid of Formula I, Formula II, or Formula III is replaced with a second side chain.

In some embodiments of the present disclosure, the acylated peptide Q comprises a spacer between the peptide and the acyl group. In some embodiments, Q is covalently bound to the spacer, which is covalently bound to the acyl group. In some exemplary embodiments, Q is modified to comprise an acyl group by acylation of an amine, hydroxyl, or thiol of a spacer, which spacer is attached to a side chain of an amino acid at position 10, 20, 24, or 29 (according to the amino acid numbering of the wild type glucagon), or at the C-terminal amino acid of the glucagon agonist peptide. The amino acid of peptide Q to which the spacer is attached can be any amino acid comprising a moiety which permits linkage to the spacer. For example, an amino acid comprising a side chain —NH₂, —OH, or —COOH (e.g., Lys, Orn, Ser, Asp, or Glu) is suitable. An amino acid of peptide Q (e.g., a singly or doubly α-substituted amino acid) comprising a side chain —NH₂, —OH, or —COOH (e.g., Lys, Orn, Ser, Asp, or Glu) is also suitable. In some embodiments the acylated glucagon agonist peptide can comprise the amino acid sequence of SEQ ID NO: 1, or a modified amino acid sequence thereof comprising one or more of the amino acid modifications described herein, with at least one of the amino acids at positions 10, 20, 24, and 29 (according to the amino acid numbering of the wild type glucagon) modified to any amino acid comprising a side chain amine, hydroxyl, or carboxylate.

In some embodiments, the spacer between the peptide Q and the acyl group is an amino acid comprising a side chain amine, hydroxyl, or thiol, or a dipeptide or tripeptide comprising an amino acid comprising a side chain amine, hydroxyl, or thiol. In some embodiments, the amino acid spacer is not γ-Glu. In some embodiments, the dipeptide spacer is not γ-Glu-γ-Glu.

When acylation occurs through an amine group of the amino acid of the spacer, the acylation can occur through the alpha amine of the amino acid or a side chain amine. In the instance in which the alpha amine is acylated, the spacer amino acid can be any amino acid. For example, the spacer amino acid can be a hydrophobic amino acid, e.g., Gly, Ala, Val, Leu, Ile, Trp, Met, Phe, Tyr. In some embodiments, the spacer amino acid can be, for example, a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, 8-aminooctanoic acid. Alternatively, the spacer amino acid can be an acidic residue, e.g., Asp and Glu. In the instance in which the side chain amine of the spacer amino acid is acylated, the spacer amino acid is an amino acid comprising a side chain amine, e.g., an amino acid of Formula I (e.g., Lys or Orn). In this instance, it is possible for both the alpha amine and the side chain amine of the spacer amino acid to be acylated, such that the peptide is diacylated. Embodiments of the invention include such diacylated molecules.

When acylation occurs through a hydroxyl group of the amino acid of the spacer, the amino acid or one of the amino acids of the dipeptide or tripeptide can be an amino acid of Formula II. In a specific exemplary embodiment, the amino acid is Ser.

When acylation occurs through a thiol group of the amino acid of the spacer, the amino acid or one of the amino acids of the dipeptide or tripeptide can be an amino acid of Formula III. In a specific exemplary embodiment, the amino acid is Cys.

In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.).

The acylated peptides Q described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In some embodiments the acylated glucagon agonist peptide can comprise SEQ ID NO: 1, including any of the modifications described herein, in which at least one of the amino acids at position 10, 20, 24, and 29 (according to the amino acid numbering of the wild type glucagon) comprise an acyl group and at least one of the amino acids at position 16, 17, 21, 24, or 29 (according to the amino acid numbering of the wild type glucagon), a position within a C-terminal extension, or the C-terminal amino acid are modified to a Cys, Lys, Orn, homo-Cys, or Ac-Phe, and the side chain of the amino acid is covalently bonded to a hydrophilic moiety (e.g., PEG). In some embodiments the acyl group is attached to position 10 (according to the amino acid numbering of the wild type glucagon), optionally via a spacer comprising Cys, Lys, Orn, homo-Cys, or Ac-Phe, and the hydrophilic moiety is incorporated at a Cys residue at position 24.

Alternatively, the acylated peptide (Q) can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

### Alkylation of Q

In some embodiments, Q is modified to comprise an alkyl group. The alkyl group can be covalently linked directly to an amino acid of the peptide Q, or indirectly to an amino acid of Q via a spacer, wherein the spacer is positioned between the amino acid of Q and the alkyl group. The alkyl group can be attached to Q via an ether, thioether, or amino linkage, for example. Q may be alkylated at the same amino acid position where a hydrophilic moiety is linked, or at a different amino acid position. As described herein, Q may comprise an alkyl group which is non-native to a naturally-occurring amino acid. In one embodiment the alkyl group is a C4 to C30 alkyl group, optionally with a carboxylate group at its free end when linked to the glucagon agonist peptide. In other embodiments, the alkyl group is a C16, C18 or C20 alkyl group optionally with a carboxylate at its free end when linked to the glucagon agonist peptide.

Alkylation can be carried out at any position within Q. Where Q is a glucagon agonist peptide, alkylation may occur at any position including any of positions 1-29, a position within a C-terminal extension, or the C-terminal amino acid, provided that an agonist activity of the unalkyated peptide is retained upon alkylation. Nonlimiting examples include alkylation at positions 5, 7, 10, 11, 12, 13, 14, 16, 17, 18, 19, 20, 21, 24, 27, 28, or 29 (according to the amino acid numbering of wild type glucagon) or at positions 30, 37, 38, 39, 40, 41, 42, or 43 of a C-terminal extended glucagon agonist peptide (according to the amino acid numbering of wild type glucagon). Other nonlimiting examples include alkylation at position 10 (according to the amino acid numbering of wild type glucagon) and pegylation at one or more positions in the C-terminal portion of the glucagon agonist peptide, e.g., position 24, 28 or 29 (according to the amino acid numbering of wild type glucagon), within a C-terminal extension, or at the C-terminus (e.g., through adding a C-terminal Cys).

In a specific aspect of the invention, peptide Q is modified to comprise an alkyl group by direct alkylation of an amine, hydroxyl, or thiol of a side chain of an amino acid of Q. In some embodiments, Q is directly alkylated through the side chain amine, hydroxyl, or thiol of an amino acid. In some embodiments, where Q is a glucagon agonist peptide, alkylation is at position 10, 20, 24, or 29 (according to the amino acid numbering of wild type glucagon). In this regard, the alkylated glucagon agonist peptide can comprise the amino acid sequence of SEQ ID NO: 1, or a modified amino acid sequence thereof comprising one or more of the amino acid modifications described herein, with at least one of the amino acids at positions 10, 20, 24, and 29 (according to the amino acid numbering of wild type glucagon) modified to any amino acid comprising a side chain amine, hydroxyl, or thiol. In some specific embodiments of the invention, the direct alkylation of Q occurs through the side chain amine, hydroxyl, or thiol of the amino acid at position 10 (according to the amino acid numbering of wild type glucagon).

In some embodiments, the amino acid of peptide Q comprising a side chain amine is an amino acid of Formula I. In some exemplary embodiments, the amino acid of Formula I, is the amino acid wherein n is 4 (Lys) or n is 3 (Orn). In other embodiments, the amino acid of peptide Q comprising a side chain hydroxyl is an amino acid of Formula II. In some exemplary embodiments, the amino acid of Formula II is the amino acid wherein n is 1 (Ser). In yet other embodiments, the amino acid of peptide Q comprising a side chain thiol is an amino acid of Formula III. In some exemplary embodiments, the amino acid of Formula II is the amino acid wherein n is 1 (Cys).

In yet other embodiments, the amino acid of peptide Q comprising a side chain amine, hydroxyl, or thiol is a disubstituted amino acid comprising the same structure of Formula I, Formula II, or Formula III, except that the hydrogen bonded to the alpha carbon of the amino acid of Formula I, Formula II, or Formula III is replaced with a second side chain.

In some embodiments of the invention, the alkylated peptide Q comprises a spacer between the peptide and the alkyl group. In some embodiments, the Q is covalently bound to the spacer, which is covalently bound to the alkyl group. In some exemplary embodiments, peptide Q is modified to comprise an alkyl group by alkylation of an amine, hydroxyl, or thiol of a spacer, which spacer is attached to a side chain of an amino acid at position 10, 20, 24, or 29 (according to the amino acid numbering of wild type glucagon) of Q. The amino acid of peptide Q to which the spacer is attached can be any amino acid comprising a moiety which permits linkage to the spacer. The amino acid of peptide Q to which the spacer is attached can be any amino acid (e.g., a singly α-substituted amino acid or an α,α-disubstituted amino acid) comprising a moiety which permits linkage to the spacer. An amino acid of peptide Q comprising a side chain —NH₂, —OH, or —COOH (e.g., Lys, Orn, Ser, Asp, or Glu) is suitable. In some embodiments the alkylated Q can comprise the amino acid sequence of SEQ ID NO: 1, or a modified amino acid sequence thereof comprising one or more of the amino acid modifications described herein, with at least one of the amino acids at positions 10, 20, 24, and 29 (according to the amino acid numbering of wild type glucagon) modified to any amino acid comprising a side chain amine, hydroxyl, or carboxylate.

In some embodiments, the spacer between the peptide Q and the alkyl group is an amino acid comprising a side chain amine, hydroxyl, or thiol or a dipeptide or tripeptide comprising an amino acid comprising a side chain amine, hydroxyl, or thiol. In some embodiments, the amino acid spacer is not γ-Glu. In some embodiments, the dipeptide spacer is not γ-Glu-γ-Glu.

When alkylation occurs through an amine group of the amino acid of the spacer the alkylation can occur through the alpha amine of the amino acid or a side chain amine. In the instance in which the alpha amine is alkylated, the spacer amino acid can be any amino acid. For example, the spacer amino acid can be a hydrophobic amino acid, e.g., Gly, Ala, Val, Leu, Ile, Trp, Met, Phe, Tyr. Alternatively, the spacer amino acid can be an acidic residue, e.g., Asp and Glu. In exemplary embodiments, the spacer amino acid can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, 8-aminooctanoic acid. Alternatively, the spacer amino acid can be an acidic residue, e.g., Asp and Glu, provided that the alkylation occurs on the alpha amine of the acidic residue. In the instance in which the side chain amine of the spacer amino acid is alkylated, the spacer amino acid is an amino acid comprising a side chain amine, e.g., an amino acid of Formula I (e.g., Lys or Orn). In this instance, it is possible for both the alpha amine and the side chain amine of the spacer amino acid to be alkylated, such that the peptide is dialkylated. Embodiments of the invention include such dialkylated molecules.

When alkylation occurs through a hydroxyl group of the amino acid of the spacer, the amino acid or one of the amino acids of the spacer can be an amino acid of Formula II. In a specific exemplary embodiment, the amino acid is Ser.

When alkylation occurs through a thiol group of the amino acid of the spacer, the amino acid or one of the amino acids of the spacer can be an amino acid of Formula III. In a specific exemplary embodiment, the amino acid is Cys.

In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.).

The alkylated peptides (Q) described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In some embodiments the alkylated Q can comprise SEQ ID NO: 1, or a modified amino acid sequence thereof comprising one or more of the amino acid modifications described herein, in which at least one of the amino acids at position 10, 20, 24, and 29 (according to the amino acid numbering of wild type glucagon) comprise an alkyl group and at least one of the amino acids at position 16, 17, 21, 24, and 29, a position within a C-terminal extension or the C-terminal amino acid are modified to a Cys, Lys, Orn, homo-Cys, or Ac-Phe, and the side chain of the amino acid is covalently bonded to a hydrophilic moiety (e.g., PEG). In some embodiments the alkyl group is attached to position 10 (according to the amino acid numbering of wild type glucagon), optionally via a spacer comprising Cys, Lys, Orn, homo-Cys, or Ac-Phe, and the hydrophilic moiety is incorporated at a Cys residue at position 24.

Alternatively, the alkylated peptide Q can comprise a spacer, wherein the spacer is both alkylated and modified to comprise the hydrophilic moiety. Nonlimiting examples of suitable spacers include a spacer comprising one or more amino acids selected from the group consisting of Cys, Lys, Orn, homo-Cys, and Ac-Phe.

### Fc Fusion Heterologous Moiety

In some embodiments Q is conjugated, e.g., fused to an immunoglobulin or portion thereof (e.g. variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC).

For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In a related embodiment, the Fc region may comprise one or more native or modified constant regions from an immunoglobulin heavy chain, other than CH1, for example, the CH2 and CH3 regions of IgG and IgA, or the CH3 and CH4 regions of IgE.

Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain.

Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Sayers et al. (J Biol Chem. 279(34):35320-5, 2004).

Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety.

### Hydrophilic Heterologous Moiety

In some embodiments, Q described herein is covalently bonded to a hydrophilic moiety. Hydrophilic moieties can be attached to Q under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the peptide by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Further activating groups which can be used to link the hydrophilic moiety (water soluble polymer) to a protein include an alpha-halogenated acyl group (e.g., alpha-iodo acetic acid, alpha-bromoacetic acid, alpha-chloroacetic acid). In specific aspects, an amino acid residue of the peptide having a thiol is modified with a hydrophilic moiety such as PEG. In some embodiments, an amino acid on Q comprising a thiol is modified with maleimide-activated PEG in a Michael addition reaction to result in a PEGylated peptide comprising the thioether linkage shown below:

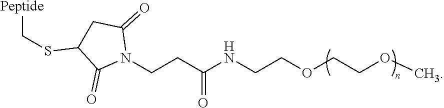

In some embodiments, the thiol of an amino acid of Q is modified with a haloacetyl-activated PEG in a nucleophilic substitution reaction to result in a PEGylated peptide comprising the thioether linkage shown below:

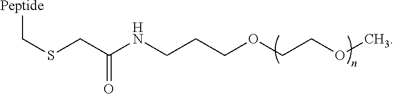

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by α1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD.

The hydrophilic moiety, e.g., polyethylene glycol chain, in accordance with some embodiments has a molecular weight selected from the range of about 500 to about 40,000 Daltons. In some embodiments the polyethylene glycol chain has a molecular weight selected from the range of about 500 to about 5,000 Daltons, or about 1,000 to about 5,000 Daltons. In another embodiment the hydrophilic moiety, e.g., polyethylene glycol chain, has a molecular weight of about 10,000 to about 20,000 Daltons. In yet other exemplary embodiments the hydrophilic moiety, e.g. polyethylene glycol chain, has a molecular weight of about 20,000 to about 40,000 Daltons.

Linear or branched hydrophilic polymers are contemplated. Resulting preparations of conjugates may be essentially monodisperse or polydisperse, and may have about 0.5, 0.7, 1, 1.2, 1.5 or 2 polymer moieties per peptide.

In some embodiments, the native amino acid of the peptide is substituted with an amino acid having a side chain suitable for crosslinking with hydrophilic moieties, to facilitate linkage of the hydrophilic moiety to the peptide. Exemplary amino acids include Cys, Lys, Orn, homo-Cys, or acetyl phenylalanine (Ac-Phe). In other embodiments, an amino acid modified to comprise a hydrophilic group is added to the peptide at the C-terminus.

In some embodiments, the peptide of the conjugate is conjugated to a hydrophilic moiety, e.g. PEG, via covalent linkage between a side chain of an amino acid of the peptide and the hydrophilic moiety. In some embodiments, the peptide is conjugated to a hydrophilic moiety via the side chain of an amino acid at position 16, 17, 21, 24, 29, 40, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG).

### Multimers

The glucagon agonist peptides, Q may be part of a dimer, trimer or higher order multimer comprising at least two, three, or more peptides bound via a linker, wherein at least one or both peptides is a glucagon related peptide. The dimer may be a homodimer or heterodimer. In some embodiments, the linker is selected from the group consisting of a bifunctional thiol crosslinker and a bi-functional amine crosslinker. In some aspects of the invention, the monomers are connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer are attached together in a “tail-to-tail” orientation in which the C-terminal amino acids of each monomer are attached together. A conjugate moiety may be covalently linked to any of the glucagon related peptides described herein, including a dimer, trimer or higher order multimer.

### Conjugates

In some embodiments, the peptides (Q) described herein are glycosylated, amidated, carboxylated, phosphorylated, esterified, N-acylated, cyclized via, e.g., a disulfide bridge, or converted into a salt (e.g., an acid addition salt, a basic addition salt), and/or optionally dimerized, multimerized, or polymerized, or conjugated.

The present disclosure also encompasses conjugates in which Q of Q-L-Y is further linked to a heterologous moiety. The conjugation between Q and the heterologous moiety can be through covalent bonding, non-covalent bonding (e.g. electrostatic interactions, hydrogen bonds, van der Waals interactions, salt bridges, hydrophobic interactions, and the like), or both types of bonding. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. In some aspects, the covalent bonds are peptide bonds. The conjugation of Q to the heterologous moiety may be indirect or direct conjugation, the former of which may involve a linker or spacer. Suitable linkers and spacers are known in the art and include, but not limited to, any of the linkers or spacers described herein under the sections “Acylation and alkylation”.

As used herein, the term “heterologous moiety” is synonymous with the term “conjugate moiety” and refers to any molecule (chemical or biochemical, naturally-occurring or non-coded) which is different from Q to which it is attached. Exemplary conjugate moieties that can be linked to Q include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising Q and a plasma protein, wherein the plasma protein is selected from the group consisting of albumin, transferin, fibrinogen and globulins. In some embodiments the plasma protein moiety of the conjugate is albumin or transferin. The conjugate in some embodiments comprises Q and one or more of a polypeptide, a nucleic acid molecule, an antibody or fragment thereof, a polymer, a quantum dot, a small molecule, a toxin, a diagnostic agent, a carbohydrate, an amino acid.

### Prodrug Derivative of the Glucagon/T3 Conjugates

In accordance with one embodiment a non-enzymatic self cleaving dipeptide moiety is provided that can be covalently linked to either the glucagon agonist peptide or the thyroid hormone receptor ligand of the glucagon/T3 conjugate, or both, wherein the dipeptide (and any compound linked to the dipeptide) is released from the conjugate at a predetermined length of time after exposure to physiological conditions. Advantageously, the rate of cleavage depends on the structure and stereochemistry of the dipeptide element and also on the strength of the nucleophile present on the dipeptide that induces cleavage and diketopiperazine or diketomorpholine formation. In one embodiment a complex comprising the glucagon/T3 conjugate and a dipeptide of the structure A-B is provided, wherein A is an amino acid or a hydroxyl acid and B is an N-alkylated amino acid that is linked to the glucagon/T3 conjugate through formation of an amide bond between B and an amine of the glucagon/T3 conjugate. The amino acids of the dipeptide are selected such that a non-enzymatic chemical cleavage of A-B from the drug produces a diketopiperazine or diketomorpholine and the reconstituted native drug.

In one embodiment a glucagon/T3 conjugate is provided comprising a complex having the general structure of A-B-(Q-L-Y) wherein

A is an amino acid or a hydroxyl acid;

B is an N-alkylated amino acid, wherein the dipeptide A-B is covalently linked to an amine (forming an amide bond) of either the glucagon agonist peptide or the thyroid hormone receptor ligand of the glucagon/T3 conjugate. In one embodiment the side chain of A or B of the dipeptide is acylated or alkylated with an hydrocarbon chain of sufficient length to bind plasma proteins. In one embodiment the dipeptide further comprises a depot polymer linked to the side chain of A or B. Chemical cleavage of A-B from Q produces a diketopiperazine or diketomorpholine and releases the active drug to the patient in a controlled manner over a predetermined duration of time after administration.

In one embodiment the dipeptide element linked to the glucagon/T3 conjugate comprises a compound having the general structure of Formula I:

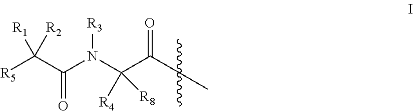

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH.

In another embodiment the dipeptide element linked to the glucagon/T3 conjugate comprises a compound having the general structure of Formula I:

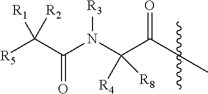

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In one embodiment a complex is provided comprising the general structure A-B-(Q-L-Y), wherein Q-L-Y comprises any of the structures as described elsewhere in this disclosure and A-B is a dipeptide that is linked via an amide bond to an amine of the Q-L-Y conjugate. In one embodiment A-B is linked to amine present on L. In one embodiment A-B is linked to amine present on Q. In one embodiment A-B is linked to amine present on Y.

In one embodiment, a complex of the structure A-B-(Q-L-Y) is provided, wherein Q-L-Y comprises any of the structures as described elsewhere in this disclosure and wherein

A is an amino acid or a hydroxy acid;

B is an N-alkylated amino acid linked to Q or Y through an amide bond between a carboxyl moiety of B and an amine of Q or Y; and

A-B comprises the structure:

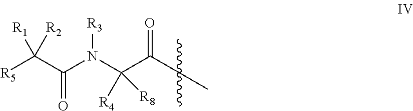

wherein
(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;
(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;
(c) R⁵ is NHR⁶ or OH;
(d) R⁶ is H, C₁-C₈ alkyl; and
(e) R⁷ is selected from the group consisting of H and OH
wherein the chemical cleavage half-life (t_{1/2}) of A-B from Q or Y is at least about 1 hour to about 1 week in PBS under physiological conditions.

In a further embodiment, A-B comprises the structure:

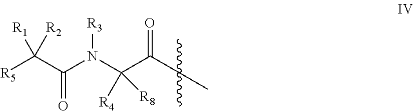

wherein

R₁ and R₈ are independently H or C₁-C₈ alkyl;

R₂ and R₄ are independently selected from the group consisting of H, C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, and (C₁-C₄ alkyl)(C₆ aryl)R₇;

R₃ is C₁-C₆ alkyl;

R₅ is NH₂; and

R₇ is selected from the group consisting of hydrogen, and OH.
In a further embodiment, A-B comprises the structure:

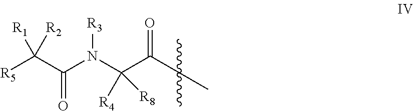

wherein

R₁ is H;

R₂ is H, C₁-C₄ alkyl, (CH₂ alkyl)OH, (C₁-C₄ alkyl)NH₂, or (CH₂)(C₆ aryl)R₇;

R₃ is C₁-C₆ alkyl;

R₄ is H, C₁-C₄ alkyl, or (CH₂)(C₆ aryl)R₇;

R₅ is NH₂;

R₈ is hydrogen; and

R₇ is H or OH.

In a further embodiment, A-B comprises the structure:

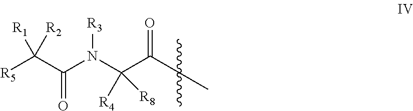

wherein

R₁ is H;

R₂ is (C₁-C₄ alkyl)NH₂;

R₃ is C₁-C₆ alkyl;

R₄ is H, C₁-C₄ alkyl, or (CH₂)(C₆ aryl)R₇;

R₅ is NH₂; and

R₈ is hydrogen.

### EXEMPLARY EMBODIMENTS

In accordance with embodiment 1 a conjugate comprising the structure Q-L-Y is provided;

wherein

Q is a glucagon agonist peptide;

Y is a thyroid receptor ligand; and

L is a linking group or a bond joining Q to Y.

In accordance with embodiment 2, the conjugate of claim 1 is provided, wherein Y is a compound having the general structure

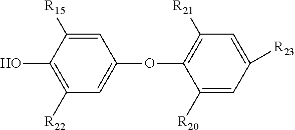

wherein

R₁₅ is C₁-C₄ alkyl, —CH₂(pyridazinone), —CH₂(OH)(phenyl)F, —CH(OH)CH₃, halo or H;

R₂₀ is halo, CH₃ or H;

R₂₁ is halo, CH₃ or H;

R₂₂ is H, OH, halo, —CH₂(OH)(C₆ aryl)F, and C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, or —OCH₂PO₃²⁻.

In accordance with embodiment 3, the conjugate of any one of claims 1 to 2 is provided wherein

wherein

R₁₅ is C₁-C₄ alkyl, —CH(OH)CH₃, I or H

R₂₀ is I, Br, CH₃ or H;

R₂₁ is I, Br, CH₃ or H;

R₂₂ is H, OH, I, or C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, or —OCH₂PO₃²⁻.

In accordance with embodiment 4, the conjugate of any one of claims 1 to 3 is provided wherein

R₁₅ is C₁-C₄ alkyl, I or H;

R₂₀ is I, Br, CH₃ or H;

R₂₁ is I, Br, CH₃ or H;

R₂₂ is H, OH, I, or C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, and —OCH₂PO₃²⁻.

In accordance with embodiment 5, the conjugate of any one of claims 1 to 4 is provided wherein Y is a compound of the general structure of Formula I:

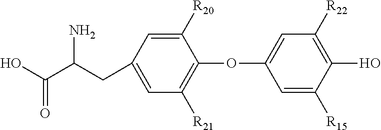

wherein

R₂₀, R₂₁, and R₂₂ are independently selected from the group consisting of H, OH, halo and C₁-C₄ alkyl; and

R₁₅ is halo or H.

In accordance with embodiment 6, the conjugate of any one of claims 1 to 5 is provided wherein Y is selected from the group consisting of 3,5,3′,5′-tetra-iodothyronine and 3,5,3′-triiodo L-thyronine.

In accordance with embodiment 7, the conjugate of any one of claims 1 to 6 is provided wherein Y is 3,5,3′-triiodo L-thyronine.

In accordance with embodiment 8, the conjugate of any one of claims 1 to 6 is provided wherein Y is a compound having the general structure

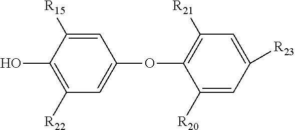

wherein

R₁₅ is isopropyl;

R₂₀ is CH₃;

R₂₁ is CH₃;

R₂₂ is H; and

R₂₃ is —OCH₂PO₃²⁻.

In accordance with embodiment 9, the conjugate of any one of claims 1 to 8 is provided wherein the conjugate comprises a glucagon agonist peptide of SEQ ID NO: 1 or analog thereof comprising at least one amino acid modification selected from the group consisting of a

substitution at position 28 with Asn, Asp, or Glu;

substitution at position 28 with Asp;

substitution at position 28 with Glu;

substitution of Thr at position 29 with a charged amino acid;

substitution of Thr at position 29 with a charged amino acid selected from the group consisting of Lys, Arg, His, Asp, Glu, cysteic acid, and homocysteic acid;

substitution at position 29 with Asp, Glu, or Lys;

substitution at position 29 with Glu or Gly;

insertion of 1-3 charged amino acids after position 29;

insertion after position 29 of Glu or Lys;

insertion after position 29 of Gly-Lys or Lys-Lys; and

Gln at position 3.

In accordance with embodiment 10, the conjugate of any one of claims 1 to 9 is provided wherein Q is a glucagon analog comprising the sequence

X₁X₂X₃GTFTSDYSX₁₂YLX₁₅X₁₆RRAQX₂₁FVX₂₄WLX₂₇X₂₈X₂₉ (SEQ ID NO: 920)

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₃ is an amino acid comprising a side chain of Structure I, II, or III:

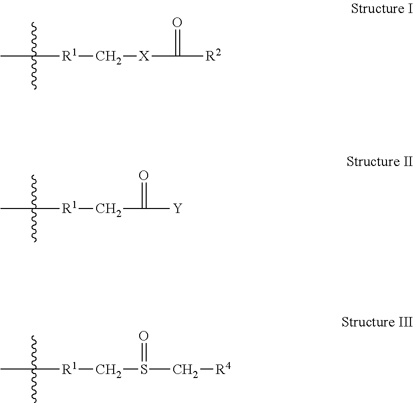

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, 0, or S; and Y is NHR⁴, SR³, or OR³;

one, two, three, or all of the amino acids at positions 16, 20, 21, and 24 substituted with an α,α-disubstituted amino acid;

X₁₂ is Lys or Arg;

X₁₅ is Asp, Glu, cysteic acid, homoglutamic acid or homocysteic acid;

X₁₆ is Ser, glutamine, homoglutamic acid, homocysteic acid, Thr or Aib;

X₂₁ is Asp, Lys, Cys, Orn, homocysteine or acetyl phenylalanine;

X₂₄ is Gln, Lys, Cys, Orn, homocysteine or acetyl phenylalanine;

X₂₇ is Met, Leu or Nle;

X₂₈ is Asn, Lys, Arg, His, Asp or Glu; and

X₂₉ is Thr, Lys, Arg, His, Gly, Asp or Glu.

In accordance with embodiment 11, the conjugate of claim 10 is provided wherein

X₃ is Gln; and

X₁₆ is Aib.

In accordance with embodiment 12, the conjugate of any one of claims 1 to 11 is provided wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) bound to amino acid 29 of the glucagon peptide through a peptide bond, wherein X₄₀ is an amino acid selected from the group consisting of Cys or Lys.

In accordance with embodiment 13, the conjugate of any one of claims 1 to 12 is provided wherein the amino acid at position 29 is Gly and the glucagon agonist peptide further comprises a C-terminal extension of SEQ ID NO: 926 (GPSSGAPPPSK).

In accordance with embodiment 14, the conjugate of any one of claims 1 to 13 is provided wherein Q is a peptide comprising the sequence of

HX₂QGTFTSDYSX₁₂YLDSRRAQDFVQWLX₂₇X₂₈GGPSSGAPPPSX₄₀ (SEQ ID NO: 924)

wherein

X₂ is selected from the group consisting of D-Ser, or Aib;

X₁₂ is Lys or Arg;

X₂₇ is Met, Leu or Nle;

X₂₈ is Asn, Lys, Arg, His, Asp or Glu; and

X₄₀ is Lys; and

Y is a compound of the general structure of Formula I:

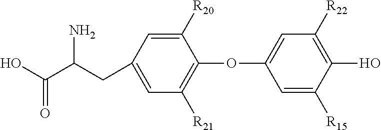

R₂₀, R₂₁ and R₂₂ are each halo and R₁₅ is H or halo.

In accordance with embodiment 15, the conjugate of any one of claims 1 to 14 is provided wherein the thyroid hormone receptor ligand is covalently attached to the side chain amine of a Lys at position 29 or at position 30-40 of a C-terminal extension relative to native glucagon.

In accordance with embodiment 16, the conjugate of any one of claims 1 to 15 is provided wherein the thyroid hormone receptor ligand is covalently attached to the side chain amine of a Lys at position 30 or 40 of said C-terminal extension.

In accordance with embodiment 17, the conjugate of any one of claims 1 to 16 is provided wherein the thyroid hormone receptor ligand is covalently attached to the glucagon agonist peptide via an amino acid or dipeptide linker.

In accordance with embodiment 18, the conjugate of any one of claims 1 to 17 is provided wherein the glucagon agonist peptide comprises the C-terminal extension of GPSSGAPPPSK (SEQ ID NO: 926); and

the thyroid hormone receptor ligand is 3,5,3′,5′-tetra-iodothyronine, or 3,5,3′-triiodo L-thyronine, wherein the thyroid hormone receptor ligand is covalently linked to the side chain amine of a Lys of the glucagon agonist peptide through a gamma glutamic acid (γGlu) spacer added to the carboxylate of the thyroid hormone receptor.

In accordance with embodiment 19, the conjugate of any one of claims 1 to 18 is provided wherein the glucagon agonist peptide comprises SEQ ID NO: 1.

In accordance with embodiment 20, the conjugate of any one of claims 1 to 19 is provided wherein L is stable in vivo, is hydrolyzable in vivo, or is metastable in vivo.

In accordance with embodiment 23, the conjugate of any one of claims 1 to 22 is provided wherein L comprises an ether moiety or an amide moiety.

In accordance with embodiment 24, the conjugate of any one of claims 1 to 19 is provided wherein Q comprises the amino acid sequence:

X₁-X₂-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Z (SEQ ID NO: 839) with 1 to 3 amino acid modifications thereto,

(a) wherein X₁ and/or X₂ is a non-native amino acid (relative to SEQ ID NO: 1601) that reduces susceptibility of the glucagon peptide to cleavage by dipeptidyl peptidase IV (DPP-IV),

(b) wherein Z is selected from the group consisting of —COOH, -Asn-COOH, Asn-Thr-COOH, and W—COOH, wherein W is selected from the group consisting of GPSSGAPPPS (SEQ ID NO: 823), GGPSSGAPPPS (SEQ ID NO: 928), GPSSGAPPPK (SEQ ID NO: 929), GGPSSGAPPPK (SEQ ID NO: 930), NGGPSSGAPPPS (SEQ ID NO: 931) and NGGPSSGAPPPSK (SEQ ID NO: 932), wherein Q exhibits glucagon agonist activity.

In accordance with embodiment 25, the conjugate of any one of claims 1 to 24 is provided wherein Q comprises the amino acid sequence of SEQ ID NO: 1 and comprises:

(a) at least one amino acid modification selected from the group consisting of:

(i) substitution of Thr at position 29 with a charged amino acid;

(ii) substitution of Thr at position 29 with a charged amino acid selected from the group consisting of Lys, Arg, His, Asp, Glu, cysteic acid, and homocysteic acid;

(iii) substitution at position 29 with Asp, Glu, or Lys;

(iv) substitution at position 29 with Glu or Gly;

(v) insertion after position 29 of 1 to 3 charged amino acids;

(vi) insertion after position 29 of Glu or Lys;

(vii) insertion after position 29 of Gly-Lys, or Lys-Lys;

(viii) substitution of Gln at position 3 with an amino acid comprising a side chain of Structure I, II, or III:

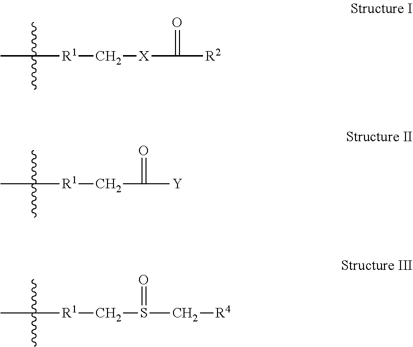

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, 0, or S; and Y is NHR⁴, SR³, or OR³; and

(ix) a combination thereof; and

(b) substitution of Ser at position 16 with Thr, Glu, or Aib; and at least one amino acid modification selected from the group consisting of:

(i) substitution of His at position 1 with a non-native amino acid that reduces susceptibility of the glucagon peptide to cleavage by dipeptidyl peptidase IV (DPP-IV),

(ii) substitution of Ser at position 2 with a non-native amino acid that reduces susceptibility of the glucagon peptide to cleavage by dipeptidyl peptidase IV (DPP-IV),

(iii) substitution of Thr at position 7 with Ile, Abu, or Val;

(iv) substitution of Gln at position 20 with Ser, Thr, Ala, Aib, Arg, or Lys;

(v) substitution of Met at position 27 with Leu or Nle;

(vi) deletion of amino acids at positions 28-29;

(vii) deletion of the amino acid at positions 29;

(viii) addition of the amino acid sequence GPSSGAPPPS to the C-terminus; and

(ix) addition of the amino acid sequence GPSSGAPPPSX to the C-terminus, wherein X is any amino acid; and

(x) a combination thereof;

wherein Q exhibits glucagon agonist activity.

In accordance with embodiment 26, the conjugate of any one of claims 1 to 25 is provided wherein L-Y is covalently conjugated to the N-terminus, C-terminus, or an amino acid side chain of Q.

In accordance with embodiment 27, the conjugate of any one of claims 1 to 26 is provided wherein L-Y is covalently conjugated to an amino acid side chain of an amino acid at position 10, 30, 37, 38, 39, 40, 41, 42, or 43 of Q, and L is an amino acid or dipeptide.

In accordance with embodiment 28, the conjugate of any one of claims 1 to 27 is provided wherein L-Y comprises the structure:

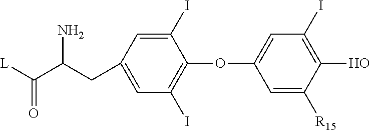

wherein

L is a bond, an amino acid, or dipeptide joining Q to Y; and

R₁₅ is H or I.

In accordance with embodiment 29, the conjugate of claim 28 is provided wherein L is γ-Glu or the dipeptide, γ-Glu-γ-Glu.

In accordance with embodiment 30, the conjugate of any one of claims 1 to 27 is provided wherein L-Y comprises the structure

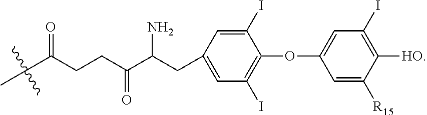

In accordance with embodiment 31, the conjugate of any one of claims 1 to 30 is provided wherein Q comprises the sequence

X₁X₂QGTFTSDYSKYLX₁₅X₁₆RRAQDFVQWLX₂₇X₂₈GGPSSGAPPPSX₄₀ (SEQ ID NO: 927)

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₁₅ is Asp, Glu, cysteic acid, homoglutamic acid or homocysteic acid;

X₁₆ is Ser, glutamine, homoglutamic acid, homocysteic acid, Thr or Aib;

X₂₇ is Met, Leu or Nle;

X₂₈ is Asn, Lys, Arg, His, Asp or Glu; and

X₄₀ is an amino acid selected from the group consisting of Cys or Lys.

In accordance with embodiment 32, the conjugate of any one of claims 1 to 31 is provided wherein L-Y is conjugated to an amino acid side chain of Q at position 40.

In accordance with embodiment 33, the conjugate of any one of claims 1 to 32 is provided wherein

X₁ is His;

X₂ is selected from the group consisting of Ser, D-Ser, Ala, D-Ala, Gly, N-methyl-Ser, Aib, Val, or α-amino-N-butyric acid;

X₁₅ is Asp, Glu, cysteic acid, homoglutamic acid or homocysteic acid;

X₁₆ is Ser, glutamine, Thr or Aib;

X₂₇ is Met, Leu or Nle;

X₂₈ is Asn;

X₂₉ is Thr or Gly; and

X₄₀ is Lys.

In accordance with embodiment 34, the conjugate of any one of claims 1 to 33 is provided further comprising the structure A-B, wherein

A is an amino acid or a hydroxy acid;

B is an N-alkylated amino acid linked to Q or Y through an amide bond between a carboxyl moiety of B and an amine of Q or Y; and

A-B comprises the structure:

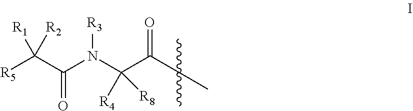

wherein

(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;

(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;

(c) R⁵ is NHR⁶ or OH;

(d) R⁶ is H, C₁-C₈ alkyl; and

(e) R⁷ is selected from the group consisting of H and OH

wherein the chemical cleavage half-life (t_{1/2}) of A-B from Q or Y is at least about 1 hour to about 1 week in PBS under physiological conditions.

In accordance with embodiment 35, the conjugate of claim 34 is provided wherein
R₁ and R₈ are independently H or C₁-C₈ alkyl;
R₂ and R₄ are independently selected from the group consisting of H, C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, and (C₁-C₄ alkyl)(C₆ aryl)R₇;
R₃ is C₁-C₆ alkyl;
R₅ is NH₂; and
R₇ is selected from the group consisting of hydrogen, and OH.

In accordance with embodiment 36, the conjugate of any one of claims 1 to 35 wherein
R₁ is H;
R₂ is H, C₁-C₄ alkyl, (CH₂ alkyl)OH, (C₁-C₄ alkyl)NH₂, or (CH₂)(C₆ aryl)R₇;
R₃ is C₁-C₆ alkyl;
R₄ is H, C₁-C₄ alkyl, or (CH₂)(C₆ aryl)R₇;
R₈ is hydrogen; and
R₅ is an amine.

In accordance with embodiment 37, the conjugate of any one of claims 1 to 36 is provided further comprising an amino acid side chain on Q covalently attached to an acyl group or an alkyl group via an alkyl amine, amide, ether, ester, thioether, or thioester linkage, which acyl group or alkyl group is non-native to a naturally occurring amino acid.

In accordance with embodiment 38, the conjugate of any one of claims 1 to 37 is provided wherein the amino acid to which the acyl or alkyl group is attached is at position 10, 20, or 24 or at position 30, 37, 38, 39, 40, 41, 32, or 43 of a C-terminal amino acid extension relative to the sequence of native glucagon.

In accordance with embodiment 39, the conjugate of claim 38 is provided wherein the amino acid to which the acyl or alkyl group is attached is at a position corresponding to position 10 relative to the sequence of native glucagon.

In accordance with embodiment 40, the conjugate of claim 38 is provided, wherein the acyl group or the alkyl group is attached to the side chain of the amino acid through a spacer and comprises carboxylate at the free end of the alkyl or acyl group.

In accordance with embodiment 41, the conjugate of any one of claims 1 to 40 is provided wherein the spacer is an acidic amino acid or an acidic dipeptide.

In accordance with embodiment 42, the conjugate of any one of claims 1 to 41 is provided as a pharmaceutical composition comprising the conjugate of any one of the known glucagon agonist peptides and a pharmaceutically acceptable carrier.

In accordance with embodiment 43, the conjugate of any one of claims 1 to 42 for use in for treating a disease or medical condition in a patient, wherein the disease or medical condition is selected from the group consisting of hyperlipidemia, metabolic syndrome, diabetes, obesity, liver steatosis, and chronic cardiovascular disease, comprising administering to the patient the pharmaceutical composition of embodiment 40 in an amount effective to treat the disease or medical condition.

### Example 1

### Generation of Glucagon and Thyroid Hormone Conjugates

Applicants designed a series of unimolecular conjugates of glucagon and thyroid hormone. First, a dipeptidyl peptidase IV (DPP-IV)-resistant, C-terminally extended glucagon analog was created that allows for the site-specific addition of thyroid hormone. Starting with the native glucagon sequence (FIG. 12A), we introduced the D-stereoisomer of serine (dSer) at position 2 to impart DPP-IV resistance. To introduce enough chemical space to facilitate the addition of thyroid hormones to the peptide without negatively impacting activity at the glucagon receptor (GcgR), we added an 11-residue C-terminal extension derived from the GLP-1 paralog exendin-4 along with a terminal lysine (Lys) residue to serve as the anchor point for thyroid hormone conjugation. This 40-mer glucagon analog is used as the “glucagon” component in the conjugates described in this Example, and has a comparable in vitro activity profile at GcgR as native glucagon (FIG. 12B).

We then constructed three different glucagon/thyroid hormone conjugates. Two of these conjugates include the most bioactive form of thyroid hormone, 3,5,3′-triiodothryonine (T3), in which the orientation of covalent attachment to the peptide is inverted relative to each other. In the first conjugate, herein referred to as “glucagon/T3”, the T3 moiety is covalently attached to the side chain amine of the C-terminal Lys40 through a gamma glutamic acid (γGlu) spacer added to the carboxylate of T3 (FIG. 12C). In the second conjugate, the T3 attachment is inverted relative to the first conjugate (glucagon/T3) and is thus herein referred to as “glucagon/iT3”. In the second conjugate, the amine of T3 is covalently linked to the peptide through a succinate spacer at Lys40 (FIG. 12D). For the third conjugate, 3,3,5′-triiodothryonine was used, otherwise called reverse T3 (rT3), an inactive metabolite of thyroid hormone. The rT3 was coupled to glucagon with the same linker chemistry as used with glucagon/T3 to generate the conjugate referred to as “glucagon/rT3” (FIG. 12E).

As expected, the three different conjugates have similar activity as the parent peptide at GcgR (FIG. 13E) yet only glucagon/T3 (1 μM) elicited transcriptional activity of a thyroid hormone response element (DR4) in the presence of TR when tested in HepG2 cells (FIG. 13F). These cells endogenously express GcgR and the conjugate uses it to enter the cell at which point the attached T3 can initiate transcription. The transcriptional activity observed with glucagon/T3 is not due to extracellular degradation as the conjugate remains intact with nearly no detectable degradation (as determined based on mass spectral analysis) in the presence of human plasma at 37° C. for up to 24 hours, which is well beyond the assay incubation period.

### Materials and Methods

### Peptide Synthesis.

Peptide backbones were synthesized by standard fluorenylmethoxycarbonyl (Fmoc)-based solid phase peptide synthesis using 0.1 mmol Rink amide 4-methylbenzhydrylamine (MBHA) resin (Midwest Biotech) on an Applied Biosystems 433A peptide synthesizer. The automated synthesizer utilized 20% piperidine in N-methyl-2-pyrrolidone (NMP) for N-terminal amine deprotection and diisopropylcarbodiimide (DIC)/6-CI-HOBt for amino acid coupling.

### Synthesis of Glucagon/T3 Conjugate.

A 1:1 molar ratio of 3, 5, 3′-triiodothyronine and di-tert-butyl dicarbonate was dissolved in dioxane/water (4:1, v:v) in the presence of an ice bath with an addition of 0.1 equivalent of triethylamine (TEA). The reaction was stirred for 30 min at 30° C. and then at room temperature for 30 hours, during which the progress of the reaction was monitored by analytical HPLC. Upon completion, the pH of the solution was lowered to 4.0 with 0.1 M hydrochloride (HCl) acid, subsequently treated it repetitively with dichloromethane (DCM) to extract desired product. The organic phase was collected, combined and evaporated in vacuum to afford crude product Boc-T3-OH with good purity.

The peptide backbone synthesized contained a C-terminal N′-methyltrityl-Llysine (Lys(Mtt)-OH) moiety, whose side chain was orthogonally deprotected by four sequential 10-min treatments with 1% trifluoroacetic acid (TFA), 2% triisopropylsilane (TIS) in DCM to expose an amine as a site for T3 conjugation. The peptidyl-resin was then mixed with a tenfold excess of Fmoc-L-Glu-OtBu(rE) activated by 3-(diethoxyphosphoryloxy)-1,2,3-benzotriazin-4(3H)-one/N,Ndiisopropylethylamine (DEPBT/DIEA) in dimethylformide (DMF) for 2 hours. The completion of the coupling was confirmed by Kaiser test, after which the resin was washed and treated with 20% piperidine in DMF to remove the Fmoc protecting group located at the side chain of the γGlu residue. Subsequently, the peptidyl-resin was reacted with a fivefold excess of crude Boc-T3-OH combined with DEPBT/DIEA in DMF for 2 hours to facilitate T3 conjugation to peptide backbone. Afterwards, the resin were treated with TFA cleavage cocktail containing TFA/anisole/TIS/H2O (85:5:5:5) for 2 hours at room temperature to release conjugate from solid support. Cleaved and fully deprotected conjugate was precipitated and washed with chilled diethyl-ether. The crude conjugates was dissolved in 15% aqueous acetonitrile containing 15% acetic acid and purified by preparative reversed-phase HPLC utilizing a linear gradient of buffer B over buffer A (A: 10% aqueous acetonitrile, 0.1% TFA; B: 100% acetonitrile, 0.1% TFA) on an axia-packed phenomenex luna C18 column (250×21.20 mm) to afford the desired conjugate with carboxyl coupling of T3 to glucagon.

### Synthesis of Glucagon/iT3 Conjugate.

3, 5, 3′-triiodothyronine (CHEM-IMPEX INT'L INC.) was solubilized in tert-butyl acetate in the presence of 0.1 equivalent of perchloric acid (HClO₄). The mixture was stirred at 0° C. for 2 hours and at room temperature for 14 hours. Upon completion, the mixture was washed with water and ethyl acetate, treated with 10M sodium hydroxide (NaOH) until pH of the solution reached 9. Subsequently, the mixture was extracted with DCM. The combined organic phase was dried by magnesium sulfate (MgSO₄) and evaporated in vacuum to obtain the desired product NH₂-T3-OtBu. NH₂-T3-OtBu and succinic anhydride were mixed in anhydrous DMF with 0.1 equivalent of DIEA. The reaction was stirred at room temperature for 48 hours. The OH-Suc-T3-OtBu product was obtained followed the same workup as described for NH2-T3-OtBu. Crude OH-Suc-T3-OtBu was dissolved in 15% aqueous acetonitrile containing 15% acetic acid and purified by semi-preparative reversed-phase HPLC using a linear gradient of buffer B over buffer A on an axia-packed phenomenex Luna C18 column (250×21.20 mm). Equimolar equivalents of HO-Suc-T3-OtBu, DEPBT and DIEA were solubilized in DMF and directly added to the peptidyl-resin. The reaction was gently agitated at room temperature for two hours and was monitored by Kaiser test. The peptidyl resins were treated with TFA cleavage cocktail containing TFA/anisole/tTIS/H2O (85:5:5:5) for 2 hours at room temperature to cleave conjugate from solid support. Cleaved conjugate was precipitated and washed with chilled diethyl-ether. The glucagon/iT3 was dissolved and purified by reversed-phase HPLC using the condition described above.

### Synthesis of Glucagon/rT3 Conjugate

A 1:1 molar ratio of 3, 3′, 5′-triiodothyronine and di-tert-butyl dicarbonate were dissolved in dioxane/water (4:1, v:v) in the presence of an ice bath with an addition of 0.1 equivalent of TEA. The reaction was stirred for 30 mins at 0° C. and at room temperature for another 30 hours. Upon completion, the pH of the solution was lowered to 4.0 with 0.1 M HCl, subsequently treated it repetitively with DCM to extract desired product. The organic phase was collected, combined and evaporated in vacuum to afford crude product Boc-rT3-OH with good purity. The peptide backbone synthesized contained a C-terminal N′-methyltrityl-Llysine (Lys(Mtt)-OH) moiety, whose side chain was orthogonally deprotected by four 10-min treatments with 1% TFA, 2% TIS in DCM to expose amine. The peptidylresin was then mixed with a tenfold excess of Fmoc-L-Glu-OtBu (γGlu) activated by DEPBT/DIEA in DMF for 2 hours. The completion of the coupling was confirmed by Kaiser test, after which the resin was washed and treated with 20% piperidine in DMF to remove the Fmoc protecting group on the γGlu residue. Subsequently, the peptidyl-resin was reacted with a fivefold excess of crude Boc-rT3-OH combined with DEPBT/DIEA in DMF for 2 hours to facilitate rT3 conjugation to peptide backbone.

Afterwards, the resin were treated with TFA cleavage cocktail containing TFA/anisole/TIS/H2O (85:5:5:5) for 2 hours at room temperature to release conjugate from solid support. Cleaved and fully deprotected conjugate was precipitated and washed with chilled diethyl-ether. The glucagon/rT3 conjugate was dissolved and purified by reversed-phase HPLC using the same condition described above. We confirmed the molecular weights of peptide and conjugates by electrospray ionization (ESI) mass spectrometry and confirmed their character by analytical reversed-phase (HPLC in 0.1% TFA with an ACN gradient on a Zorbax C8 column (0.46 cm×5 cm).

### Human Glucagon Receptor Activation.

Each peptide or conjugate was individually tested for its ability to activate the human GcgR through a cell-based luciferase reporter gene assay that indirectly measures cAMP induction. Human embryonic kidney (HEK293) cells were co-transfected with GcgR cDNA (zeocin-selection) and a luciferase reporter gene construct fused to a cAMP response element (CRE) (hygromycin B-selection). Cells were seeded at a density of 22,000 cells per well and serum deprived for 16 h in DMEM (HyClone) supplemented with 0.25% (vol/vol) bovine growth serum (BGS) (HyClone). Serial dilutions of the peptides were added to 96-well cell-culture treated plates (BD Biosciences) containing the serum-deprived, co-transfected HEK293 cells, and incubated for 5 h at 37° C. and 5% CO2 in a humidified environment. To stop the incubation, an equivalent volume of Steady Lite HTS luminescence substrate reagent (Perkin Elmer) was added to the cells to induce lysis and expose the lysates to luciferin. The cells were agitated for 5 min and stored for 10 min in the dark.

Luminescence was measured on a MicroB eta-1450 liquid scintillation counter (Perkin-Elmer). Luminescence data was graphed against concentration of peptide and EC50 values were calculated using Origin software (OriginLab).

### TR Transcriptional Activity.

For testing of transcriptional activity HEPG2 cells were cultured in HAMF12/DEMEM medium (Biochrom), supplemented with 10 FBS. Cells were seeded at a density of 5×104 cells/well in a 96 well plate. One day after seeding cells were transfect each with 0.45 ng of DR4-luciferase and TRalpha plasmids using Mefatektene (Biontex). Two days after transfection cells were stimulated with 1 μM of each compound for 10 hours. Reaction was stopped and luciferase activity was measured according to the manufactures protocol (Promega).

### Plasma Stability.

Each compound was incubated with phosphate buffered saline (PBS, pH=7.4) containing 60% mouse plasma at 37° C. for the duration of the study. At the time points of 6 h, 24 h and 72 h, aliquots of the incubated solutions were withdrawn and diluted with acetonitrile to precipitate the plasma proteins, which were subsequently removed by microcentrifugation at 13,000 rpm for 5 mins. The supernatant was collected and diluted for the analytical reversed-phase HPLC using a linear gradient of buffer B over buffer A (A: 10% aqueous acetonitrile, 0.1% TFA; B: 90% aqueous acetonitrile, 0.1% TFA) on a Zorbax C8 column (4.6×50 mm). RNA-Seq Total RNAs were extracted from frozen liver samples of 4 independent mice per group (vehicle, glucagon only, T3 only, co-administration of glucagon and T3 and glucagon/T3) using TriPure RNA reagent (Sigma). Poly-A isolation, library generation and amplification were performed according to the protocol of the mRNASeq Library Prep Kit (Lexogen). The cDNA libraries were then converted to 5500 W librairies using the 5500 W Conversion Primer Kit (Lexogen). The Barcoded cDNA libraries were sequenced on a SOLiD 5500×1 Wildfire sequencer (Life technology). Mapping of color-coded reads on the mouse genome (mm10 assembly) was performed using the Lifescope software (Life Technologies). Reads for annotated (Ensembl) genes were counted using HtSeq. Normalization of expression levels for each gene and differential expression analysis were performed using DES eq276 (Bioconductor R package, false discovery rate <5%). The synergy score (SS) was calculated as the relative fold change (FC) of (glucagon/T3) compared to the maximum FC of T3 and glucagon alone: SS=FC (glucagon/T3)/max (T3, glucagon). Synergistic hits were selected by a SS >1.5. To reduce noise we removed transcripts from the hit list witch do not show significant regulation p<0.05 (BH corrected) when treated with (glucagon/T3) or opposite regulation.

### Pathway Enrichment.

Enriched KEGG pathways were determined using the hypergeometric distribution test using MATLAB (R2015b). Significant gene regulation was determined using DESeq2. Regulated genes used for pathway enrichment were selected using a threshold of p<0.01 and the false detection rate corrected using Benjamini & Hochberg (BH) procedure. Among significant enriched KEGG pathways, relevant pathways were manually selected.

### Wild-Type Mice for Pharmacology Studies.

For studies on lipid handling in wildtype mice (Western mice), male C57Bl/6j mice (Jackson Laboratories) were fed a atherogenic Western diet (Research Diets D12079B), which is a high-cholesterol diet (0.21% gm %) with 41% kcal from fat, 43% kcal from carbohydrates, and 17% kcal from protein. For studies on energy metabolism in obese mice (DIO mice), male C57Bl/6j mice (Jackson Laboratories) were fed a diabetogenic diet (Research Diets D12331), which is a high-sucrose diet with 58% kcal from fat 25.5% kcal from carbohydrates, and 16.4% kcal from protein. Both dietary challenges began at 8 weeks of age. HFHSD and HFHCD mice were single- or group-housed on a 12:12-h light-dark cycle at 22° C. with free access to food and water. Mice were maintained under these conditions for a minimum of 16 weeks before initiation of pharmacological studies and were between the ages of 6 months to 12 months old. All injections and tests were performed during the light cycle. Compounds were administered in a vehicle of 1% Tween-80 and 1% DMSO and were given by daily subcutaneous injections at the indicated doses at a volume of 5 μl per g body weight. Mice were randomized and evenly distributed to test groups according to body weight and body composition. If ex vivo molecular biology/histology/biochemistry analyses were performed, the entire group of mice for each treatment was analyzed and scored in a blinded fashion.

### Genetically-Modified Mouse Lines.

Liver-specific Thrb−/− mice were generated by crossing Thrbflox/flox mice with Alfp-Cre mice. Thrbflox/flox; Cre negative littermates were used as wild-type controls. Mice were maintained on the HFHCD for 8 weeks prior to initiation of treatment. A follow-up study was conducted 4 weeks after the start of the first arm to confirm the effects. The data presented is a compilation of the two independent studies. Inducible, global Gcgr−/− mice were generated by crossing Gcgrflox/flox mice with Rosa26-Cre-ERT2 (tamoxifen-inducible) mice. Design and construction of the Gcgr targeting vector and the subsequent steps to generate mice heterozygous of Gcgrflox/+ were performed by the Gene Targeted Mouse Service Core at the University of Cincinnati. Briefly, the vector was designed to “flox” exons 4-10 of the Gcgr gene, with the neomycin resistant gene and one loxP site being inserted in the intron upstream of exon 4 and the other loxP site in the intron downstream of exon 10. The “floxed” region and the two homologous arms, 3.4 kb and 2.5 kb respectively, were PCR-amplified from mouse genomic DNA and cloned into the vector. The construct was sequenced and then electroporated into mouse ES cells derived from a C57B16 strain, and the resulting cells were subject to drug selection on media containing G418. Drug resistant clones were initially screened by PCR and further confirmed by Southern blot analysis. Correctly targeted ES cell clones were injected into albino blastocysts to generate chimeras, which were then bred with C57B16 female mice to obtain ES cell-derived offspring as determined by the presence of black coat color.

Black mice were further analyzed by PCR for transmission of targeted Gcgr gene. The neomycin cassette was deleted by breeding with mice carrying “Flip” recombinase.

Gcgrflox/+ mice lacking the neomycin cassette and Flip allele were selected by subsequent breeding to wild-type C57B16 mice. The mice were backcrossed to C57B16 background for 5 generations and the crossed with Rosa26-Cre-ERT2 mice (Gt(ROSA)26Sortm1(cre/ERT2)Tyj), obtained from The Jackson Laboratory (Stock number #008463).

Gcgrflox/flox; Rosa26-Cre-ERT2 mice were maintained on the HFHSD for 12 weeks prior to induction of knockdown via twice daily interaperitoneal injections with tamoxifen (1 mg in 100 μl) for 5 consecutive days. Mice that received oil injections were used as wild-type controls. Treatment with compounds were initiated after 2 weeks of washout and recovery following the last tamoxifen injection.

Global Ldlr−/− mice and wild-type littermates were purchased from Jackson laboratories and were maintained on the HFHCD for 12 weeks prior to treatment initation. Global Ucp1−/− mice and wild-type littermates were bred in house, housed at 30° C., and maintained on a HFHSD for 12 weeks prior to initiations of treatment. All mice were single- or group-housed on a 12:12-h light-dark cycle with free access to food and water.

### Rodent Pharmacological and Metabolism Studies.

Compounds were administered by repeated subcutaneous injections in the middle of the light phase at the indicated doses with the indicated durations. Co-administration of compounds was administered by single formulated injections. Body weights and food intake were measured every day or every other day after the first injection. All studies with wild-type mice were performed with a group size of n=8 or greater using mice on a C57B16j background.

For assessment of glucose, pyruvate, and insulin tolerance during chronic treatment, the challenge tests were performed at least 24 hours after the last administration of compounds. The investigators were not blinded to group allocation during the in vivo experiments or to the assessment of longitudinal endpoints. All rodent studies were approved by and performed according to the guidelines of the Institutional Animal Care and Use Committee of the Helmholtz Center Munich, University of Cincinnati, Universite de Lyon, and in accordance with guidelines of the Association for the Assessment and Accreditation of Laboratory and Animal Care (AAALAC # Unit Number: 001057) and appropriate federal, state and local guidelines, respectively.

### Body Composition Measurements.

Whole-body composition (fat and lean mass) was measured using nuclear magnetic resonance technology (EchoMRI).

### Indirect Calorimetry.

Energy intake, energy expenditure, respiratory exchange ratio, and home-cage activity were assessed using a combined indirect calorimetry system (TSE Systems). O₂ consumption and CO₂ production were measured every 10 min for a total of up to 120 h (after 24 h of adaptation) to determine the respiratory quotient and energy expenditure after an initial treatment regimen. Food intake was determined continuously for the same time as the indirect calorimetry assessments by integration of scales into the sealed cage environment. Home-cage locomotor activity was determined using a multidimensional infrared light beam system with beams scanning the bottom and top levels of the cage, and activity being expressed as beam breaks.

### Blood Parameters.

Blood was collected at the indicated times from tail veins or after euthanasia using EDTA-coated microvette tubes (Sarstedt), immediately chilled on ice, centrifuged at 5,000 g and 4° C., and plasma was stored at −80° C. For fast liquid performance chromatography (FPLC) of cholesterol distribution in different lipoprotein fractions, fresh plasma from each treatment group was pooled (n=6-8) and ran over two Superose 6 HR columns in tandem. Cholesterol levels in the collected fractions were determined by colorimetric assay. Plasma insulin and T3 were quantified by an ELISA assay (Ultrasenstive Mouse Insulin ELISA and Rodent T3 ELISA; Alpco). Plasma FGF21 was quantified by an ELISA assay (Mouse FGF21 ELISA; Millipore). Plasma cholesterol, extracted hepatic cholesterol, triglycerides, ALT, and AST were measured using enzymatic assay kits (Thermo Fisher). Plasma creatinine and blood urea nitrogen were measured using enzymatic assay kits (Abcam). Plasma free fatty acids were measured using enzymatic assay kits (Wako). All assays were performed according to the manufacturers' instructions.

### Histopathology.

After chronic treatment, HFHCD-fed or HFHSD-fed C57B16/j male mice (age) were sacrificed with CO2, body weight as well as heart weights and tibia length was taken during necropsy. Livers and whole hearts were embedded in paraffin using a vacuum infiltration processor TissueTEK VIP (Sakura). 3 μm thick slides were cut using a HMS35 rotatory microtome (Zeiss) and H & E staining was performed. For H & E staining, rehydration was done in a decreasing ethanol series, rinsing with tapwater, 2 min Mayers acid Hemalum, bluing in tapwater followed by lmin EosinY (both BioOptica). Dehydration was performed in increasing ethanol series, mounting with Pertex® (Medite GmbH) and coverslips (CarlRoth Chemicals). The slides were evaluated independently using a brightfield microscope (Axioplan, Zeiss). Photos were taken using the Hamamatsu-Nanozoomer HT2.0 in 1.25×, 5×. 20× and 40× magnification. The hepatic steatosis score is defined as the unweighted sum of the three individual scores for steatosis, lobular inflammation and ballooning degeneration. Steatosis is graded by the presence of fat vacuoles in liver cells according to the percentage of affected tissue (0: <5%; 1: 5-33%; 2: 33-66%; 3: >66%). Lobular inflammation is scored by overall assessment of inflammatory foci per 200× field (0: no foci; 1: <2 foci; 2: 2-4 foci; 3: >4 foci). The individual score for ballooning degeneration ranges from 0 (none), 1 (few cells) to 2 (many cells). Total scores range from 0 to 8 with scores <2 considered non-steatosis, 3 considered as borderline steatosis, 4-5 considered onset of steatosis, and >6 considered steatosis. Different to liver and heart, inguinal fat pad samples were embedded in paraffin using Leica embedding machine (EG1150 H) and cut in 5 μm sections using Leica Microtome (RM2255) to perform H&E staining. Samples were stained with hematoxyline for 4 minutes and eosinY for 2 minutes and fixed with Roti®-Histokitt (Carl Roth) before analysing them independently using Microscope Scope A.1 (Zeiss).

### Echocardiography.

For transthoracic echocardiography, a Vevo2100 Imaging System (VisualSonics Inc., Toronto Canada) with a 30 MHz probe was used. All echocardiograms were performed on conscious animals to prevent anesthesia-related impairment of cardiac function as reported previously. Briefly, echocardiograms were obtained in parasternal long and short axis views. For accurate linear measurements of LV internal dimensions (LVID) and parasternal (LVPW) or septal (IVS) wall thicknesses, M-mode images of the heart in parasternal short-axis view at the level of the papillary muscle were acquired. Qualitative and quantitative measurements were made offline using analytical software (VisualSonics Inc.). Fractional shortening (FS) was calculated as FS %=[(LVIDd−LVIDs)/LVIDd]×100. Ejection fraction (EF) was calculated as EF %=100*((LVvolD−LVvolS)/LVvolD) with LVvol=((7.0/(2.4+LVID)*LVID³). The corrected LV mass (LV MassCor) was calculated as LV MassCor=0.8(1.053*((LVIDd+LVPWd+IVSd)³−LVIDd³)). Heart rate and respiration rate were determined from M-mode tracings, using 3 consecutive intervals.

### Rectal Body Temperature:

Rectal body temperature was measured in conscious mice using a high-precision thermometer (thermosensor: Almemo ZA 9040, data logger: Almemo 2290-8, Ahlborn, Holzkirchen, Germany) that was carefully inserted into the rectum. Temperature of each individual was taken by the same researcher one hour after lights-on after chronic treatment ab libitum.

### Atherosclerotic Plaque Assessment.

The extent of atherosclerotic lesion formation was assessed in aortic root sections of Ldlr−/− mice that were treated with glucagon/T3 or vehicle for 2 weeks by staining for lipid depositions with oil-red O. Briefly, atherosclerotic lesions were measured in 4-μm transverse cryo sections of aortic roots. Images of tissue sections were taken (Leica analysis software LAS) and quantified by manually outlining the lumen boundary. Subsequently, oil red O+ areas were outlined as well and the percentage of oil-red O+ areas in relation to the total area was calculated.

### Immunohistochemistry for UCP1.

iWAT samples were dissected and subsequently fixed and stored in 4% paraformaldehyde. After dehydration, tissues were embedded in paraffin and cut in 5□m sections to perform immunohistochemistry using rabbit anti-UCP1 antibody (Abcam, ab10983). Therefore, samples were deparaffinized and microwaved in citrate buffer (pH=6) for antigen retrival. To quench endogenous peroxidases samples were incubated with 3% hydrogen peroxide in methanol and then blocked with Avidin D, Biotin (Vectastain ABC Kit; Vector labs) and normal goat serum (10%). Anti-UCP1 antibody (1:400) was added and incubated overnight, before applying secondary anti-rabbit antibody (1:300; Vector Labs ZA0324).

Vectastain ABC reagent (Vectastain ABC Kit; Vector labs) was used followed by application of SIGMAFAST 3,3′-Diaminobenzidine (Sigma) for signal development, and subsequent counterstaining with hematoxylin and mounting. Finally, sections were analyzed using Microscope Scope A.1 (Zeiss).

Quantification of accumulated T3 in the liver and iWAT.

The stock solutions of T3 (Sigma Aldrich) and stable isotope-labeled T3 ([13C6]T3; Cambridge Isotope Laboratories) were prepared dissolving 5 mg of the standard in 100 mL of pure MeOH. T3 calibration standards that ranged in concentration from 0.5 pg/μL to 100 pg/uL were prepared from stock solution through dilution with a solvent mixture of 20% acetonitrile in water. [13C6]T3 was prepared in a mixture of 20% acetonitrile in water at a concentration of 10 pg/μL. The calibration solutions as well as the internal standard solutions, were protected from light, stored at 4° C. and wrapped with aluminum foil.

Tissue samples were homogenized in methanol containing an antioxidant solution (ascorbic acid, citric acid, and dithiothreitol at 25 g/L in methanol) by ultrasonication (Bandelin Electronics) 2×20 s under cooling with ice. Liquid-liquid extraction was performed as described in 77. Solid phase extraction was performed loaded the water phase into a SampliQ SPE cartridge (60 mg, 3 mL; Agilent Technologies), which were preconditioned sequentially with 3 mL of 50% a methanol in chloroform, 3 mL of pure methanol and 3 mL of water. The target compound as well as the internal standard were eluted with 0.6 mL of 0.1% formic acid in methanol. The solvent was evaporated to dryness under N2 steam and then reconstituted in 0.3 mL of 0.1N HCl in water. The thyroid hormone derivatives were extracted back in organic solvent with 3×0.3 mL of ethyl acetate. The solvent was evaporated again and compounds re-dissolved in 60 μL in a mixture of 20% acetonitrile in water for instrumental analysis.

Compound separation was carried out on a nanoAcquity UHPLC system (Waters Corporation) interfaced with a quadrupole time-of-flight mass spectrometer Q-TOF2 (Waters-Micromass). The system was operated under MassLynx 4.1 software (Waters-Micromass) in QTOF-MS mode. Samples were infused at a flow rate of 5 μL/min and were monitored in positive ion electrospray mode. High purity nitrogen was used as de-solvation and auxiliary gas; argon was used as the collision gas. The de-solvation gas was set to 200 L/h at a temperature of 120° C., the cone gas was set to 50 L/h and the source temperature at 100° C. The capillary extraction and the cone voltages were set to 2.6 kV and 35 V respectively. The QTOF detector (MPC) was operated at 2100 V. The instrumentation ran in full-scan mode with the QTOF data being collected between m/z 100-1000 with a collision energy of 6 eV. The data were collected in the continuum mode with a scan time 1.5 s, interscan delay of 0.1 s. The processing of calibration and quantification data including peak integration, internal standard correction and linear regression was carried out using the QuanLynx Application Manager (Waters-Micromass).

A 5 μL volume of tissue sample was directly injected into an HSS-T3 microscale column: 300 μm i. d.×150 mm length, 1.8 μm particle size (Waters Corporation) at a flow rate of 5 μL/min. The mobile phase was 0.1% formic acid in water (mobile phase A) and 0.1% formic acid in acetonitrile (mobile phase B). Gradient elution was performed according to the following elution program: 0-3 min, 95% A, 5% B; 3.5 min 70% A, 30% B; 5.5-6.5 min 62% A, 38% B; 7-10 min, 60% A, 40% B; 12-13 min 100% B, 13.5-20 min 95% A, 5% B. The temperature of the HSS-T3 column was kept at 40° C.

### Gene Expression Analysis.

Gene expression profiling in the liver, iWAT, eWAT, BAT, and heart were performed following treatment of mice according to the treatment paradigms explained in the figure legends for each specific analsysis. For tissue collection, mice were fasted for 4 h and treated with compounds 2 h prior to tissue collection. Gene expression was profiled with quantitative real-time RT-PCR using either TaqMan single probes or with specifically-designed TaqMan low-density array cards. The relative expression of the selected genes was normalized to the reference gene hypoxanthine-guanine phosphoribosyltransferase (Hprt).

### Results

### Glucagon/T3 Synergistically Improves Hepatic Cholesterol and Lipid Handling

To determine whether glucagon-mediated delivery of T3 can reverse hypercholesterolemia, hypertriglyceridemia, and hepatic steatosis in metabolically compromised mice, we administered the glucagon/T3 conjugates, along with monoagonist controls, to mice maintained on a high-fat, high-cholesterol diet (HFHCD).

After treating mice for two weeks with single daily injections at a dose of 100 nmoles/kg, the amount of T3 observed in the liver increased following treatment with glucagon/T3 (FIG. 1A), indicating that the liver is a primary site of action. Glucagon/T3 reduced circulating levels of total cholesterol (FIG. 1B) as well as the fraction of cholesterol bound to both low-density lipoproteins (LDL) and high-density lipoproteins (HDL) (FIG. 1C) to a similar extent as systemic administration of unconjugated T3. Furthermore, glucagon/T3 reduced circulating levels of triglycerides to a similar extent as the unconjugated glucagon analog (FIG. 1D).

Importantly, at sub-threshold doses for glucagon to lower cholesterol and T3 to lower triglycerides, glucagon/T3 is equally capable of lowering both lipids at an equimolar dose. Glucagon/T3 also lowered hepatic cholesterol content (FIG. 1E) and hepatocellular vacuolation (FIG. 1F) compared to vehicle controls, essentially reversing hepatic steatosis. None of the treatment groups negatively affected liver function, as confirmed by normal plasma levels of alanine aminotransferase (ALT) and aspartate aminotransferase (AST) (FIG. 14A). Renal function was unchanged, as confirmed by normal plasma levels of urea nitrogen (FIG. 14B) and creatinine (FIG. 14C). These beneficial effects of glucagon/T3 on hypercholesterolemia and hypertriglyceridemia were similar to the effects observed after equimolar coadministration of glucagon and T3 (FIG. 15A-B), demonstrating that both glucagon and T3-mediated actions are responsible for the combined effects on lipids of the conjugate. Notably, the cholesterol lowering effect, which is mostly attributable to T3 action, was not evident after treatment with glucagon/iT3 or glucagon/rT3 (FIG. 15A-B). This demonstrates the importance of the specific form of thyroid hormone and its molecular orientation for eliciting the coordinated hormonal actions.

We analyzed the livers from these treated obese mice for genes involved in cholesterol and lipid metabolism (FIG. 1G). Transcriptional profiling revealed that glucagon/T3 increased the expression of key genes involved in cholesterol metabolism (Srebp2 and Cyp7a1) and cholesterol uptake (Ldlr and Scarb1). Such gene program changes recapitulate the pleotropic molecular signature previously implicated in the cholesterol-lowering effects independently mediated by glucagon and T3.

Glucagon/T3 also increased gene programs indicative of triglyceride formation (Dgat) and lipolysis (Lipc), indicating that glucagon and thyroid hormone signaling converge in the liver to jointly induce lipid futile cycling. Moreover, glucagon/T3 triggered the expression of fatty acid oxidation-related genes (Ppara and Cpt1a), supporting the reversal of hepatic steatosis induced by the conjugate. Glucagon/T3 increased Fgf21 expression and increased circulating levels of FGF21 (FIG. 1G-H), as both glucagon and T3 are reported to convey certain metabolic actions through FGF21. Cumulatively, these changes in gene programs related to lipid metabolism reflect integrated actions resulting in decreased circulating levels and reduced hepatic deposition of cholesterol and triglycerides, thereby promoting healthy liver function in obesity.

### Glucagon/T3 Improves Lipid Handling in Ldlr−/− Mice

Murine cholesterol is primarily stored in HDL whereas human cholesterol is stored and transported in both HDL and LDL. To test in a murine model that more closely resembles human physiology, we used low-density lipoprotein receptor knockout mice (Ldlr−/−) to assess the mechanism and translational relevance of the glucagon/T3 conjugate-induced reduction of circulating cholesterol levels.

Cholesterol lowering of glucagon/T3 was confirmed in Ldlr−/− mice (FIG. 1I). Importantly, the conjugate primarily lowered cholesterol stored in the very low-density lipoprotein (VLDL) and LDL fractions without influencing HDL levels (FIG. 1J). Beyond the translational relevance, this finding indicates that complementary mechanisms independent of LDLR-mediated uptake contribute to the overall efficacy in cholesterol-lowering, which is in agreement with reports on the effects of TRβ-selective agonists to lower cholesterol in Ldlr−/− mice48.

### Glucagon/T3 Ameliorates Atherosclerosis in Ldlr−/− Mice

Since Ldlr−/− mice display atherosclerotic plaque development and aortic lesions similar to human pathophysiology, and these are not evident in wild-type mice after prolonged exposure to HFHCD, we explored whether glucagon/T3 can reverse atherosclerotic plaque formation in Ldlr−/− mice. In this restorative treatment paradigm in which the mice were maintained on HFHCD for 16 weeks, 2 weeks of treatment with glucagon/T3 reduced atherosclerotic plaque size and lesion coverage at the aortic root compared to vehicle-treated controls (FIG. 1K). Taken together, these results demonstrate that glucagon/T3 improves lipid metabolism to an extent that leads to the regression of established atherosclerosis. These findings serve as the foundation for translational studies aiming to assess the impact of long-term treatment with glucagon/T3 on chronic cardiovascular disease (CVD) caused by dyslipidemia and atherosclerosis.

### Lipid Handling Benefits are Co-Mediated by GcgR and TRI3

To exclude off-target effects and examine the contribution of each component in glucagon/T3 to the lipid lowering effects, we administered glucagon/T3 to global GcgR knockout mice (GcgR−/− mice) as well as to liver-specific thyroid hormone receptor beta knockout mice (liver-specific Thrb−/−). In GcgR−/− mice, the effects to lower cholesterol (FIG. 2A) and triglycerides (FIG. 2B) were completely lost when compared to wild-type mice. The complete loss of an effect in global GcgR−/− mice confirmed the target specificity of the conjugate, and demonstrated that glucagon activity is essential for sufficient T3 delivery and for coordinating the improved lipid handling. Furthermore, the lack of effects in GcgR−/− mice demonstrates that the T3 moiety is not prematurely separating from the peptide in circulation.

Similar to the effects in GcgR−/− mice, the effects to lower cholesterol (FIG. 2C) and triglycerides (FIG. 2D) were lost in liver-specific Thrb−/− mice. The absence of such effects in these mice demonstrates the liver selectivity of glucagon/T3, at least as it pertains to cholesterol and triglyceride metabolism. Furthermore, our results demonstrate that TRβ is responsible for mediating the effects on lipid handling and that hepatic TRα is of lesser significance. This aligns with reports that TRβ is the predominant thyroid hormone receptor regulating hepatic lipid handling.

Glucagon-Targeted T3 Adjusts Hepatic Gene Programs More Efficiently than Individual Agonists

Based on the results of the targeted transcriptomics (FIG. 1G), we performed unbiased transcriptional profiling (mRNA-seq) of livers from mice maintained on HFHCD that were treated with glucagon/T3 for 14 days. Glucagon/T3 regulated the expression of 956 genes with at least a twofold change in expression compared to vehicle (FIG. 3A). Not surprisingly, based on the results in FIG. 1, such mapping identified “steroid hormone biosynthesis” and “metabolic pathways” as two functional patterns enriched in genes that were differentially regulated in the livers of mice treated with glucagon/T3 when compared to vehicle (FIG. 3B). The subcategories of “metabolic pathways” that were enriched included many specific gene programs involved in lipid and carbohydrate metabolism (FIG. 3B). Our analysis of the transcriptomic response also uncovered 359 genes that were regulated by T3 alone and 242 genes that only responded to glucagon (FIG. 3A). Importantly, 577 genes were similarly regulated by glucagon/T3 and the co-administration of equimolar glucagon and T3 (FIG. 3A). This substantial overlap demonstrates that both glucagon-sensitive and T3-sensitive signaling events are being elicited in the liver by the glucagon/T3 conjugate.

The magnitude of the regulation of those T3-sensitive genes (which are those 359 targets identified above as T3-sensitive) is stronger with the conjugate compared to T3 alone (FIG. 3C & 3D). This suggests that the conjugate is more efficient than equimolar systemic T3 at augmenting the thyroid hormone response in the liver. This enhanced efficiency may be the result of increased accumulation of T3 in the liver arising from glucagon-mediated selective targeting (FIG. 1A), but may also be a consequence of glucagon and T3 synergism to regulate gene expression. Indeed, we observe that the conjugate synergistically regulates gene expression of certain targets (FIG. 3D), meaning that the conjugate elicits similar coordinated gene regulation patterns in a more effective manner when compared with the co-administration of glucagon and T3. To quantify this, we applied a synergy score (see methods) on just those targets that are regulated in the same direction by the conjugate and co-administration. This independent analysis shows that for 208 genes, the magnitude of regulation is conspicuously greater with the conjugate than with glucagon and T3 co-administration, supporting the notion that cumulative targeting may translate to enhanced therapeutic impact. Interestingly, 272 genes were uniquely regulated by glucagon/T3, but did not respond to single or coadministration of glucagon and T3, suggesting that novel signaling cues are being induced by the conjugate.

### Glucagon/T3 Lowers Body Weight by Increasing Energy Expenditure

Since both glucagon and T3 have been reported to increase energy expenditure and decrease body fat, we explored the weight-lowering capacity of glucagon/T3 in diet-induced obese (DIO) mice maintained on a high-fat, high-sugar diet (HFHSD) compared to mono-agonist controls at equimolar doses. Neither glucagon nor T3 appreciably lowered body weight. However, glucagon/T3 significantly lowered body weight at an equimolar dose with a 10% absolute decrease from baseline after a week of daily treatment (FIG. 4A). Importantly, glucagon/iT3 and glucagon/rT3 failed to lower body weight and did not lower lipids (FIG. 15A-C). The loss of body weight caused by glucagon/T3 was due to a loss of fat mass, not lean mass (FIG. 4B). Food intake was increased by systemic T3 treatment, recapitulating the hyperphagia associated with central hyperthyroidism in rodents, yet was not increased by glucagon/T3 (FIG. 4C). Despite the difference in energy intake, both T3 alone and glucagon/T3 substantially increased whole-body energy expenditure (FIG. 4D). However, the hyperphagia following treatment with T3 compensated for increased energy expenditure, while treatment with glucagon/T3 drove a negative energy balance resulting in a loss of body fat.

Furthermore, systemic T3 significantly increased home cage activity (FIG. 4E), which paralleled the observed increase in energy expenditure. Unlike mono-therapy with T3 however, the conjugate did not cause an increase in ambulatory activity (FIG. 4E), demonstrating that altered activity is not contributing to the enhanced energy expenditure caused by the conjugate. These data suggest that the glucagon carrier restricts T3 action to select tissues, particularly away from brain circuits governing food intake and locomotion. Consistent with these observations, treatment with T3 but not the conjugate resulted in an increase in circulating levels of T3 (FIG. 4F) and increased rectal temperature after chronic treatment (FIG. 4G). This suggests the enhanced energy expenditure elicited by glucagon/T3 is not causing a hyperthermic response. Additionally, glucagon/T3 caused a decrease in the respiratory exchange ratio (RER) (FIG. 4H) in the absence of change in food intake, indicating that the coordinated action of the two constituents shifted nutrient partitioning to promote fat utilization. Similar to the lack of cholesterol and triglyceride lowering effects observed in GcgR−/− mice, the effects of glucagon/T3 to lower body weight (FIG. 4I), enhance energy expenditure (FIG. 4J), and promote fat utilization (FIG. 4K) are absent in GcgR−/− mice. These findings clearly demonstrate that glucagon is required to unleash the targeted metabolic actions of thyroid hormone. Here, in the absence of the GcgR cellular gateway, the covalent attachment of T3 to glucagon inactivates thyroid hormone pharmacology.

Glucagon/T3 Induces the Browning of Inguinal White Fat Based on the indirect calorimetry results and because GcgR is abundantly expressed in white adipose tissue (WAT)51, we next tested the effects of glucagon/T3 on WAT and thermogenesis. Although the levels of T3 residing in iWAT are below the limits of detection in a basal state, chronic treatment with glucagon/T3 delivered a detectable amount of T3 in iWAT (FIG. 5A). The conjugate increased the multilocular nature of inguinal WAT (iWAT) and reduced adipocyte size to a similar extent as systemic T3 whereas glucagon alone had negligible effects at this dose (FIG. 5B). Much like T3 itself, glucagon/T3 turned on thermogenic gene programs in iWAT, including, Ucp1, Dio2, and Pgc1a (FIG. 5C), and triggered an increase of UCP-1 immunoreactivity in iWAT (FIG. 5D). In classical brown adipose tissue (BAT), glucagon/T3 had minimal effects on the thermogenic gene profile (FIG. S5), which is consistent with reports of a lack of direct thermogenic effects on BAT by pharmacological glucagon33 and thyromimetics. Collectively, these results demonstrate that the glucagon/T3 conjugate induces browning of iWAT and increases the thermogenic capacity of iWAT.

To test whether the observed iWAT browning is primary or secondary to fat mass lowering and changes in energy expenditure observed with glucagon/T3, we tested the conjugate in uncoupling protein-1 knockout mice (Ucp1−/−) maintained on a HFHSD. The body weight lowering of glucagon/T3 was blunted, but not completely silenced in Ucp1−/− mice compared to wild-type controls (FIG. 5D). Similar to the effects on body weight, the magnitude of the shift in RER (FIG. 5E) and the increase in energy expenditure (FIG. 5F) induced by glucagon/T3 were diminished in Ucp1−/− mice when compared to wild-type mice. Since these effects were attenuated but not completely lost, we conclude that UCP-1 dependent thermogenesis contributes to the whole-body energy metabolism benefits induced by glucagon/T3, while other mechanisms, such as futile cycling in the liver, contribute additional benefits in response to treatment with glucagon/T3.

### Concurrent T3 Activity Neutralizes the Diabetogenic Action Profile of Glucagon

Despite the well-recognized body weight benefits of chronic glucagon action, its therapeutic utility for chronically treating obesity is compromised by its well-known promotion of hepatic glucose production. Therefore, we sought to test whether targeted and integrated T3 action could counteract the adverse effects of glucagon to prevent impairment of glycemic control in DIO mice. The addition of the T3 moiety to glucagon dampened acute hyperglycemia (FIG. 6A) and chronically prevented the development of glucose intolerance or hyperglycemia (FIG. 6B), which were evident with glucagon treatment alone.

In fact, the net result of glucagon/T3 on glucose tolerance is intermediate to the two individual treatments and is substantially improved compared to vehicle-treated controls (FIG. 6B). Furthermore, glucagon/T3 improved insulin sensitivity (FIG. 6C) to a magnitude that is intermediate to glucagon and T3 alone, and lowers plasma levels of insulin (FIG. 6D), which could be the result of hepatic lipid depletion. We used a pyruvate tolerance test as an indirect measure of hepatic glucose output. As expected, the glucagon analog alone worsened pyruvate tolerance whereas T3 alone substantially improved the effect. The attached T3 moiety of the conjugate was capable of completely offsetting the gluconeogenic effects of glucagon due to direct liver targeting (FIG. 6E).

Furthermore, the attached T3 moiety prevented the glucagon-mediated increase in RER, thus limiting the shift to more carbohydrate utilization that is evident with the glucagon alone (FIG. 6F). This appears to be a direct result of the incorporated T3 action to lessen the surge in hepatic glucose production combined with increased fatty acid utilization induced by the glucagon component, as evident by the acute decrease in circulating free fatty acids induced by glucagon alone and glucagon/T3 (FIG. 6G).

Both gluconeogenic gene programs (G6pc and Pck1) and glycolytic gene programs (Gck and Pk1r) are increased by glucagon/T3 in the liver (FIG. 6H), supporting that futile glucose cycling is being engaged by coordinated glucagon and thyroid hormone signaling, and may contribute to the carbon turnover measured by indirect calorimetry. The gluconeogenic actions of glucagon are partly governed by engaging the peroxisome proliferator receptor gamma coactivator-1 (PGC-1) axis53, acting to increase PGC-1α levels and repress PGC-1β levels. Herein, we show the concurrent T3 action within the glucagon/T3 conjugate mitigates the glucagon-mediated increase in Pgc1a mRNA levels and simultaneously prevents the glucagon-mediated suppression of Pgc1b mRNA levels (FIG. 6I). Together, these reciprocal changes in PGC-1 isoform expression appear to represent the molecular underpinnings for the dampened gluconeogenic actions and enhanced fatty acid oxidation induced by the coordinated glucagon and T3 actions.

### Glucagon-Mediated T3 Delivery Prevents Cardiovascular Thyrotoxicity

Although thyroid hormones offer numerous benefits for metabolism, their therapeutic use in obesity or diabetes is undermined by deleterious effects on the cardiovascular system. Although GcgR is expressed in certain cardiovascular tissues, its expression levels are minimal compared to the levels of expression in the liver and fat depots. We assessed the impact of glucagon/T3 on cardiac hypertrophy in DIO mice using echocardiography (Echo) after 4 weeks of treatment (All Echo parameters in Table Si). Equimolar systemic T3 mono-therapy reduced heart rate (FIG. 7A) and increased respiration rate (FIG. 7B), thus indicating a damaging effect on overall respiratory capacity and efficiency. Glucagon/T3 had no effect on either parameter.

Furthermore, systemic T3 reduced both fraction shortening (FIG. 7C) and ejection fraction (FIG. 7D), demonstrating that untargeted T3 has a detrimental effect on left ventricular function. Once again, the conjugate had no apparent effect on cardiac performance (FIG. 7C-D). Along these lines, systemic T3 but not glucagon/T3 increased the tibia length-corrected heart weight (FIG. 7E) and planar crosssectional area. This demonstrates that the impaired cardiac performance by systemic T3 is the result of substantial cardiac hypertrophy, which appreciably is not evident with glucagon-mediated targeting. Notably, the cardiac hypertrophy observed with T3 alone arises from increases in diastolic (FIG. 7F) and systolic (FIG. 7G) left ventricular wall thickness, a pathological process that does not occur with glucagon/T3 treatment. Systemic T3 directly increased the expression of T3-sensitive genes (Dio2 and Ucp2) (FIG. 7H) and hypertrophic gene markers (Nppa and Nppb) (FIG. 7I) in the whole heart, which importantly did not appear to be regulated by glucagon/T3. This proves that the T3 linked to glucagon, at the dose tested, has limited entry into cardiomyocytes such that the detrimental effects on heart function are not evident.

Histological profiling of the hearts revealed that T3-treated mice displayed marked features of thyrotoxic cardiomyopathy, including larger cardiomyocytes, increased fat deposition, and infiltration of fibroblast and inflammatory cell into interstitial tissue. In addition, marked cell death was detectable in the hearts of T3-treated mice, including both single cell necrosis as well as larger infarction. Notably, none of these manifestations of thyrotoxicity or thyroid hormone induced cardiomyopathy were detectable following treatment with the glucagon/T3 conjugate at the same molar dose, which is the dose that shows profound and comprehensive improvements in metabolism.

The data demonstrates that glucagon-mediated targeting spares the cardiovascular system from direct thyroid hormone action. Notably, chronic therapy with glucagon/T3 is not associated with cardiac hypertrophy, altered ventricular function, or cardiomyocyte necrosis, all of which were observed with an equimolar treatment with T3 alone. Conversely, glucagon/T3 causes mobilization and utilization of triglycerides and cholesterol, and prevents the accumulation of atherosclerotic plaques in the aortic root, all of which are vital to reduce CHD risk. Another compelling link to FGF21 action can be made as it protects from cardiac hypertrophy and it is plausible that the observed induction of FGF21 contributes to the cardiac profile after chronic treatment with glucagon/T3. Importantly, the synergistic effects of glucagon and T3 co-agonism translate to less reliance on individual signaling cues to have equal potency as the single hormones. Thus lower circulating concentrations of the conjugate are needed to elicit lipid lowering and body weight-lowering effects, which presumably contribute to the enhanced safety profile.

The data presented herein demonstrate that combined actions derived from glucagon and thyroid hormone that are incorporated into a single molecule synergize to produce robust effects on lipid and energy metabolism, with an enhanced therapeutic index. Glucagon-mediated targeting offers an alternative to designing isoform-selective thyromimetics, which has proven difficult due to structural similarities in the binding pocket of TR isoforms.

### Example 2

The ability of each peptide to induce cAMP was measured in a firefly luciferase-based reporter assay. The cAMP production that is induced is directly proportional to the glucagon fragment binding to the glucagon receptor or GIP receptor or GLP-1 receptor. HEK293 cells co-transfected with the receptor and luciferase gene linked to a cAMP responsive element were employed for the bioassay.

The cells were serum-deprived by culturing 16 hours in Dulbecco-modified Minimum Essential Medium (Invitrogen, Carlsbad, Calif.) supplemented with 0.25% Bovine Growth Serum (HyClone, Logan, Utah) and then incubated with serial dilutions of glucagon fragments for 5 hours at 37° C., 5% CO₂ in 96 well poly-D-Lysine-coated “Biocoat” plates (BD Biosciences, San Jose, Calif.). At the end of the incubation, 100 μL of LucLite luminescence substrate reagent (Perkin Elmer, Wellesley, Mass.) were added to each well. The plate was shaken briefly, incubated 10 min in the dark and light output was measured on MicroBeta-1450 liquid scintillation counter (Perkin-Elmer, Wellesley, Mass.). The effective 50% concentrations (EC₅₀) and inhibitory 50% concentrations (IC₅₀) were calculated by using Origin software (OriginLab, Northampton, Mass.). All EC₅₀s and IC₅₀s are reported in nM, unless indicated otherwise.

1. A conjugate comprising the structure Q-L-Y;
wherein
Q is a glucagon agonist peptide comprising
A) the sequence
X₁X₂X₃GTFTSDYSX₁₂YLX₁₅X₁₆RRAQX₂₁FVX₂₄WLX₂₇X₂₈X₂₉ (SEQ ID NO: 920)
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₃ is an amino acid comprising a side chain of Structure I, II, or III: 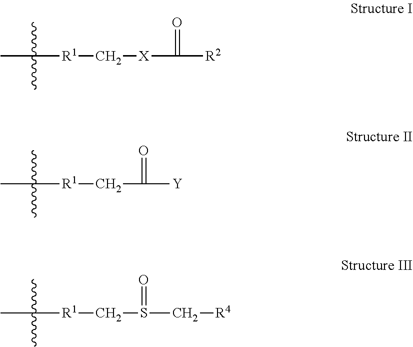
wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, 0, or S; and Y is NHR⁴, SR³, or OR³;
one, two, three, or all of the amino acids at positions 16, 20, 21, and 24 substituted with an α,α-disubstituted amino acid;
X₁₂ is Lys or Arg;
X₁₅ is Asp, Glu, cysteic acid, homoglutamic acid or homocysteic acid;
X₁₆ is Ser, glutamine, homoglutamic acid, homocysteic acid, Thr or Aib;
X₂₁ is Asp, Lys, Cys, Orn, homocysteine or acetyl phenylalanine;
X₂₄ is Gln, Lys, Cys, Orn, homocysteine or acetyl phenylalanine;
X₂₇ is Met, Leu or Nle;
X₂₈ is Asn, Lys, Arg, His, Asp or Glu; and
X₂₉ is Thr, Lys, Arg, His, Gly, Asp or Glu; or
B) the sequence
X₁X₂QGTFTSDYSKYLX₁₅X₁₆RRAQDFVQWLX₂₇X₂₈GGPSSGAPPPSX₄₀ (SEQ ID NO: 927)
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₁₅ is Asp, Glu, cysteic acid, homoglutamic acid or homocysteic acid;
X₁₆ is Ser, glutamine, homoglutamic acid, homocysteic acid, Thr or Aib;
X₂₇ is Met, Leu or Nle;
X₂₈ is Asn, Lys, Arg, His, Asp or Glu; and
X₄₀ is an amino acid selected from the group consisting of Cys or Lys; or
C) the sequence
HX₂QGTFTSDYSX₁₂YLX₁₅X₁₆RRAQDFVQWLX₂₇X₂₈X₂₉ (SEQ ID NO: 922)
wherein
X₂ is selected from the group consisting of Ser, D-Ser, Ala, D-Ala, Gly, N-methyl-Ser, Aib, Val, or α-amino-N-butyric acid;
X₁₂ is Lys or Arg;
X₁₅ is Asp or Glu;
X₁₆ is Ser, Thr or Aib,
X₂₇ is Met, Leu or Nle;
X₂₈ is Asn, Lys, Arg, His, Asp or Glu; and
X₂₉ is Thr, Lys, Arg, His, Gly, Asp or Glu, wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) bound to amino acid 29 of the glucagon peptide through a peptide bond, wherein X₄₀ is an amino acid selected from the group consisting of Cys or Lys;
Y is a thyroid receptor ligand having the general structure of:
I) 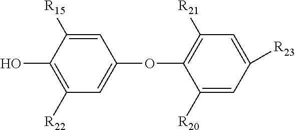
wherein
R₁₅ is C₁-C₄ alkyl, —CH₂(pyridazinone), —CH₂(OH)(phenyl)F, —CH(OH)CH₃, halo or H;
R₂₀ is halo, CH₃ or H—
R₂₁ is halo, CH₃ or H—
R₂₂ is H, OH, halo, —CH₂(OH)(C₆ aryl)F, or C₁-C₄ alkyl; and
R₂₃ is —CH₂CH(NH₂)COOH, —OCH₂COOH, —NHC(O)COOH, —CH₂COOH
—NHC(O)CH₂COOH, —CH₂CH₂COOH, and —OCH₂PO₃²⁻; or
II) 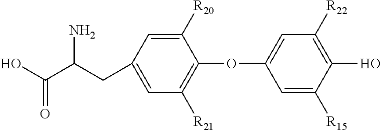
wherein
R₂₀, R₂₁ and R₂₂ are independently selected from the group consisting of H, OH, halo and C₁-C₄ alkyl; and
R₁₅ is halo or H; or
III) 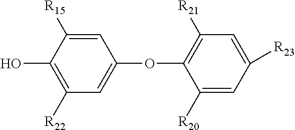
wherein
R₁₅ is C₁-C₄ alkyl, I or H;
R₂₀ is I, Br, CH₃ or H—
R₂₁ is I, Br, CH₃ or H—
R₂₂ is H, OH, I, or C₁-C₄ alkyl; and
R₂₃ is —CH₂CH(NH₂)COOH, —OCH₂COOH, —NHC(O)COOH, —CH₂COOH
—NHC(O)CH₂COOH, —CH₂CH₂COOH, and —OCH₂PO₃¹; and
L is a linking group or a bond joining Q to Y.

2-5. (canceled)

6. The conjugate of claim 1 wherein Y is selected from the group consisting of 3,5,3′,5′-tetra-iodothyronine and 3,5,3′-triiodo L-thyronine.

7. The conjugate of claim 1, wherein Y is 3,5,3′-triiodo L-thyronine.

8-9. (canceled)

10. The conjugate of claim 6 wherein Q is a glucagon analog comprising the sequence
X₁X₂X₃GTFTSDYSX₁₂YLX₁₅X₁₆RRAQX₂₁FVX₂₄WLX₂₇X₂₈X₂₉ (SEQ ID NO: 920)
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₃ is Gln 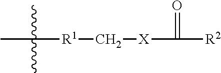 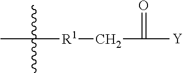 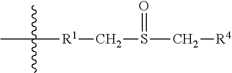
one, two, three, or all of the amino acids at positions 16, 20, 21, and 24 substituted with an α,α-disubstituted amino acid;
X₁₂ is Lys or Arg;
X₁₅ is Asp, Glu, cysteic acid, homoglutamic acid or homocysteic acid;
X₁₆ is Ser, glutamine, homoglutamic acid, homocysteic acid, Thr or Aib;
X₂₁ is Asp, Lys, Cys, Orn, homocysteine or acetyl phenylalanine;
X₂₄ is Gln, Lys, Cys, Orn, homocysteine or acetyl phenylalanine;
X₂₇ is Met, Leu or Nle;
X₂₈ is Asn, Lys, Arg, His, Asp or Glu; and
X₂₉ is Thr, Lys, Arg, His, Gly, Asp or Glu.

11. (canceled)

12. The conjugate of claim 10 wherein the glucagon agonist peptide further comprises a C-terminal extension of SEQ ID NO: 26 (GPSSGAPPPSX₄₀), SEQ ID NO: 27 (KRNRNNIAX₄₀) or SEQ ID NO: 28 (KRNRX₄₀) bound to amino acid 29 of the glucagon peptide through a peptide bond, wherein X₄₀ is an amino acid selected from the group consisting of Cys or Lys.

13. The conjugate of claim 12 wherein the amino acid at position 29 is Gly and the glucagon agonist peptide further comprises a C-terminal extension of SEQ ID NO: 926 (GPSSGAPPPSK).

14. A conjugate comprising the structure Q-L-Y;
wherein Q is a peptide comprising the sequence of
HX₂QGTFTSDYSX₁₂YLDSRRAQDFVQWLX₂₇X₂₈GGPSSGAPPPSX₄₀ (SEQ ID NO: 924)
wherein
X₂ is selected from the group consisting of D-Ser, or Aib;
X₁₂ is Lys or Arg;
X₂₇ is Met, Leu or Nle;
X₂₈ is Asn, Lys, Arg, His, Asp or Glu; and
X₄₀ is Lys; and
Y is a compound of the general structure of Formula I: 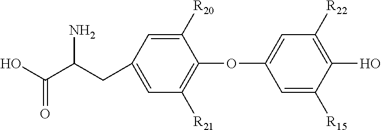
R₂₀, R₂₁ and R₂₂ are each halo and R₁₅ is H or halo wherein the thyroid hormone receptor ligand is covalently attached to the side chain amine of the Lys at X₄₀ of a Q.

15-16. (canceled)

17. The conjugate of claim 14 wherein the thyroid hormone receptor ligand is covalently attached to the glucagon agonist peptide via an amino acid or dipeptide linker.

18. The conjugate of claim 17 wherein the the thyroid hormone receptor ligand is 3,5,3′,5′-tetra-iodothyronine, or 3,5,3′-triiodo L-thyronine, wherein the thyroid hormone receptor ligand is covalently linked to the side chain amine of a Lys of the glucagon agonist peptide through a gamma glutamic acid (γGlu) spacer added to the carboxylate of the thyroid hormone receptor.

19-23. (canceled)

24. The conjugate of claim 1, wherein Q comprises the amino acid sequence:
X₁-X₂-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Z (SEQ ID NO: 839) with 1 to 3 amino acid modifications thereto,
(a) wherein X₁ is selected from the group consisting of His, D-His, N-methyl-His, alpha-methyl-His, imidazole acetic acid, des-amino-His, hydroxyl-His, acetyl-His, homo-His, and alpha, alpha-dimethyl imidiazole acetic acid (DMIA), and X₂ is selected from the group consisting of Ser, D-Ser, D-Ala, Gly, N-methyl-Ser, Val, and alpha, amino isobutyric acid (Aib), wherein at least one of X₁ and X₂ is a non-native amino acid at that position relative to SEQ ID NO: 1,
(b) wherein Z is selected from the group consisting of —COOH, -Asn-COOH, Asn-Thr-COOH, and W—COOH, wherein W is selected from the group consisting of GPSSGAPPPS (SEQ ID NO: 823), GGPSSGAPPPS (SEQ ID NO: 928), GPSSGAPPPK (SEQ ID NO: 929), GGPSSGAPPPK (SEQ ID NO: 930), NGGPSSGAPPPS (SEQ ID NO: 931) and NGGPSSGAPPPSK (SEQ ID NO: 932),
wherein Q exhibits glucagon agonist activity.

25-26. (canceled)

27. The conjugate of claim 1, wherein L-Y is covalently conjugated to an amino acid side chain of an amino acid at position 10, 30, 37, 38, 39, 40, 41, 42, or 43 of Q, and L is an amino acid or dipeptide.

28. The conjugate of claim 24, wherein L-Y comprises the structure: 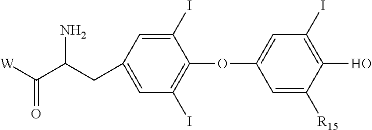
wherein
W is a bond, an amino acid, or dipeptide joining L-Y to Q; and
R₁₅ is H or I.

29. The conjugate of claim 28 wherein W is γ-Glu or the dipeptide, γ-Glu-γ-Glu.

30. The conjugate of claim 1 wherein L-Y comprises the structure 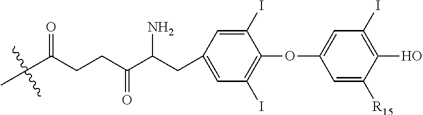
wherein R₁₅ is H or I.

31. (canceled)

32. The conjugate of claim 10, wherein the glucagon agonist peptide comprises SEQ ID NO: 1 and L-Y is conjugated to an amino acid side chain of Q at position 40.

33. The conjugate of claim 32, wherein
X₁ is His;
X₂ is selected from the group consisting of Ser, D-Ser, Ala, D-Ala, Gly, N-methyl-Ser, Aib, Val, or α-amino-N-butyric acid;
X₁₅ is Asp, Glu, cysteic acid, homoglutamic acid or homocysteic acid;
X₁₆ is Ser, glutamine, Thr or Aib;
X₂₇ is Met, Leu or Nle;
X₂₈ is Asn;
X₂₉ is Thr or Gly; and
X₄₀ is Lys.

34. A derivative of the conjugate of claim 1 further comprising the structure A-B, wherein
A is an amino acid or a hydroxy acid;
B is an N-alkylated amino acid linked to Q or Y through an amide bond between a carboxyl moiety of B and an amine of Q or Y; and
A-B comprises the structure: 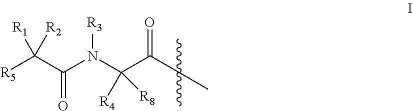
wherein
(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;
(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;
(c) R⁵ is NHR⁶ or OH;
(d) R⁶ is H, C₁-C₈ alkyl; and
(e) R⁷ is selected from the group consisting of H and OH
wherein the chemical cleavage half-life (t_{1/2}) of A-B from Q or Y is at least about 1 hour to about 1 week in PBS under physiological conditions.

35-36. (canceled)

37. The conjugate of claim 1, further comprising an amino acid side chain on Q, at a position corresponding to position 10, 20, or 24 of native glucagon, or at position 30, 37, 38, 39, 40, 41, 32, or 43 of a C-terminal extended glucagon analog, or the C-terminal amino acid, covalently attached to an acyl group or an alkyl group via an alkyl amine, amide, ether, ester, thioether, or thioester linkage, which acyl group or alkyl group is non-native to a naturally occurring amino acid.

38-41. (canceled)

42. A pharmaceutical composition comprising the conjugate of claim 1, and a pharmaceutically acceptable carrier.

43. A method for treating a disease or medical condition in a patient, wherein the disease or medical condition is selected from the group consisting of hyperlipidemia, metabolic syndrome, diabetes, obesity, liver steatosis, and chronic cardiovascular disease, comprising administering to the patient the pharmaceutical composition of claim 42 in an amount effective to treat the disease or medical condition.
